(19) United States
(12) Patent Application Publication
(10) Pub. No.: US 2010/0319083 A1
(43) Pub. Date: Dec. 16, 2010

(54) Method of producing plants having enhanced transpiration efficiency and plants produced therefrom

(75) Inventors: Josette Masle, Queanbeyan (AU); Graham Douglas Farquhar, Queanbeyan (AU); Scott Robert Gilmore, Downer (AU)

Correspondence Address: COOPER & DUNHAM, LLP, 30 Rockefeller Plaza, 20th Floor, NEW YORK, NY, 10112, US

(73) Assignee: THE AUSTRALIAN NATIONAL UNIVERSITY

(21) Appl. No.: 12/588,000

(22) Filed: Oct. 14, 2009

Related U.S. Application Data
(63) Continuation of application No. 10/591,135, filed on Aug. 30, 2006, now Pat. No. 7,408,312.
Application No. 10/591,135 is a continuation of application No. 10/519,135, filed on Aug. 15, 2005, now abandoned.

(30) Foreign Application Priority Data
Jul. 2, 2002 (AU) PS-3339

(51) Int. Cl. A01H 1/00; C12Q 1/68

(52) U.S. Cl. 800/278; 435/6

(57) ABSTRACT

The present invention provides methods of selecting plants having modified transpiration efficiency using plant ERECTA gene sequences and nucleic acids linked thereto, and to methods of producing plants having modified transpiration efficiency using isolated plant ERECTA gene sequences, in both traditional plant breeding and genetic engineering approaches. The invention further extends to plants produced by the methods described.

This application is a continuation of U.S. Ser. No. 10/519,135, filed Aug. 15, 2005, a §371 National Stage of PCT International Application No. PCT/AU2003/000854, filed Jul. 2, 2003, claiming priority of Australian Patent Application No. PS 3339, filed Jul. 2, 2003, the contents of which are hereby incorporated by reference,

### FIELD OF THE INVENTION

The present invention relates to the field of plant breeding and the production of genetically engineered plants. More specifically, the invention described herein provides genes that are capable of enhancing the transpiration efficiency of a plant when expressed therein. These genes are particularly useful for the production of plants having enhanced transpiration efficiency, by both traditional plant breeding and genetic engineering approaches. The invention further extends to plants produced by the methods described herein.

### BACKGROUND TO THE INVENTION

1. General

This specification contains nucleotide and amino acid sequence information prepared using Patent In Version 3.1, presented herein after the claims. Each nucleotide sequence is identified in the sequence listing by the numeric indicator <210> followed by the sequence identifier (e.g. <210>1, <210>2, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer is obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purposes of exemplification only. Functionally equivalent products, compositions and methods are clearly within the scope of the invention, as described herein.

2. Description of the Related Art

It is well known that virtually all plants require a certain quantity of water for proper growth and development, because CO₂ fixation and photosynthate assimilation by plants cost water. A significant quantity of water absorbed by plants from the soil returns to the atmosphere via plant transpiration.

Transpiration efficiency is a measure of the amount of dry matter produced by a plant per unit of water transpired, or, in other words, carbon gain relative to water lost through transpiration.

For plants having low transpiration efficiency, or when water is in short supply, the loss of water through transpiration can limit key metabolic processes associated with plant growth and development. For example, during drought, or when plants having low transpiration efficiency are grown in arid and semi-arid environments, plant productivity as determined by dry matter production or photosynthetic rate, is considerably reduced. Accordingly, the production of plants having enhanced water use efficiency or transpiration efficiency is highly desirable for their adaptation to arid or semi-arid conditions, or to enhance their drought resistance.

The enhancement of water use efficiency or transpiration efficiency by plants is also highly desirable in consideration of global climatic change and increasing pressure on world water resources. The inefficient utilization of agricultural water is known to impact adversely upon the supply of navigable water, potable water, and water for industrial or recreational use. Accordingly, the production of plants having enhanced transpiration efficiency is highly desirable for reducing the pressure on these water resources. It is also desirable for increasing plant productivity under well-watered conditions.

By enhancing transpiration efficiency, carbon gain rates are enhanced per unit of water transpired, thereby stimulating plant growth under well-watered conditions, or alternatively, under mild or severe drought conditions. This is achieved by enhancing carbon gain more than transpiration rate, or by reducing the amount of water lost at any particular rate of carbon fixation. Those skilled in the art also consider that for a given growth rate plants having enhanced transpiration efficiency dry out soils more slowly, and use less water, than less efficient near-isogenic plants.

Several chemical as well as environmental pre-treatments have been described for enhancing the ability of plant seedlings to survive drought, either by reducing transpiration or by reducing the amount of water that is actually lost to the atmosphere.

Known environmental treatments largely involve the use of physical barriers. Whilst placing a physical barrier over plant stomata is known to reduce water loss via transpiration, the procedure is not always desirable or practicable for field-grown crops. For example, physical barriers over plant stomata may inhibit certain gas-exchange processes of the plant. It is more desirable to enhance actual transpiration efficiency or water use efficiency of the plant through manipulation of intrinsic plant function.

Chemical agents are typically the so-called “anti-transpirant” or “anti-desiccant” agents, both of which are applied to the leaves. Anti-transpirants are typically films or metabolic anti-transpirants.

These products form a film on leaves, thereby either blocking stomatal pores, or coating leaf epidermal cells with a water-proof film. Typical film anti-transpirants include waxes, wax-oil emulsions, higher alcohols, silicones, plastics, latexes and resins. For example, Elmore, U.S. Pat. No. 4,645,682 disclosed an anti-transpirant consisting of an aqueous paste wax; Cushman et al., U.S. Pat. Nos. 3,791,839 and 3,847,641 also disclosed wax emulsions for controlling transpiration in plants; and Petrucco et al., U.S. Pat. No. 3,826,671, disclosed a polymer composition said to be effective for controlling transpiration in plants.

Metabolic anti-transpirants generally close stomata, thereby reducing the rate of transpiration. Typical metabolic anti-transpirants include succinic acids, phenylmercuric acetate, hydroxysulfonates, the herbicide atrazine, sodium azide, and phenylhydrazones, as well as carbon cyanide.

Compounds having plant growth regulator activity have also been shown to be useful for reducing transpiration. For example, Bliesner et al., U.S. Pat. No. 4,671,816, disclosed an acetylene compound, said to possess utility for regulating plant growth, whilst Kuznetsov et al. (Russian Patent No. SU 1,282,492, Russian Patent Application No. SU 1,253,559-A1), and Smirnov et al (Russian Patent No. SU 1,098,934) disclosed the use of derivatives of 2-methyl-5-hydroxybenzimidazole, and the chloride or bromide salts thereof, as anti-transpirant growth regulators. Vichnevetskaia (U.S. Pat. No. 5,589,437 issued Dec. 31, 1996) also describe hydroxybenzimidazole derivatives for enhancing the drought resistance of plants by reducing transpiration. Schulz et al., U.S. Pat. No. 4,943,315, also disclosed formulations comprising an acetylene and a phenylbenzylurea compound, for reducing transpiration in plants and/or for avoiding impairment to plants caused by heat and dry conditions. Abscisic acid has also been shown to reduce or suppress transpiration in plants (eg. Helv. Chim. Acta, 71, 931, 1988; J. Org. Chem., 54, 681, 1989; and Japanese Patent Publication No. 184,966/1991).

Metabolic anti-transpirants are costly to produce and often exhibit phytotoxic effects or inhibit plant growth (Kozlowski (1979), In: Tree Growth and Environmental Stresses (Univ. of Washington Press, Seattle and London)), and are not practically used.

Recent studies have examined alternative methods for enhancing transpiration efficiency, particularly breeding approaches to select lines that grow more efficiently under mild drought conditions. Carbon isotope discrimination has been used to identify Arabidopsis ecotypes with contrasted transpiration efficiencies (Masle et al., In: Stable isotopes and plant carbon-water relations, Acad. Press, Physiol. Ser., pp 371-386, 1993) and to assist conventional breeding of new plant varieties in a number of species (Hall et al., Plant Breeding Reviews 4, 81-113, 1994) including rice (Farquhar et al., In: Breaking the Yield Barrier, ed K G Cassman, IRRI, 95, 101) and most recently wheat (Rebetzke et al. Crop Science 42:739-745, 2002).

No single gene has been identified as being capable of enhancing transpiration efficiency when expressed in planta. Transpiration efficiency may well be multigenic. As a consequence, the genes and signalling pathways that regulate the photosynthetic and/or stomatal components of the transpiration efficiency mechanism in plants have not been identified or characterized.

Moreover, notwithstanding that the effect of down-regulating expression of the Rubisco gene, or mutation in genes involved in abscisic acid (eg. aba, abi), are known to modify transpiration efficiency to some extent through stomatal closure, the consequence of such modifications is not necessarily specific, resulting in pleiotropic effects.

Arabidopsis thaliana ecotype Landsberg erecta (L-er1) is one of the most popular ecotypes and is used widely for both molecular and genetic studies. It harbors the er1 mutation, which confers a compact inflorescence, blunt fruits, and short petioles. There are a number of erecta mutant alleles. Phenotypic characterization of the mutant alleles suggests a role for the wild type ER gene in regulating plant morphogenesis, particularly the shapes of organs that originate from the shoot apical meristem. Torii et al., The Plant Cell 8, 735, 1996, showed that the ER gene encodes a putative receptor protein kinase comprising a cytoplasmic protein kinase catalytic domain, a transmembrane region, and an extracellular domain consisting of leucine-rich repeats, which are thought to interact with other macromolecules.

### SUMMARY OF THE INVENTION

In work leading up to the present invention, the inventors sought to elucidate the specific genetic determinants of plant transpiration efficiency. In plants, the development of molecular genetic markers, such as, for example, genetic markers that map to a region of the genome of a crop plant, such as, for example, a region of the rice genome, maize genome, barley genome, sorghum genome, or wheat genome, or a region of the tomato genome or of any Brassicaceae, assists in the production of plants having enhanced transpiration efficiency (Edwards et al., Genetics 116, 113-125, 1987; Paterson et al., Nature 335, 721-726, 1988).

The present inventors identified a locus that is linked to the genetic variation in transpiration efficiency in plants. To elucidate a locus associated with the transpiration efficiency of plants, the inventors established experimental conditions and sampling procedures to determine the contribution to total transpiration efficiency of the factors influencing this phenotype, and, more particularly, the genetic contribution to the total variation in transpiration efficiency. Factors influencing transpiration efficiency include, for example, genotype of the plant, environment (eg. temperature, light, humidity, boundary layer around the leaves, root growth conditions), development (eg. age and/or stage and/or posture of plants that modify gas exchange and/or carbon metabolism), and seed-specific factors (Masle et al. 1993, op. cit). The screens developed by the inventors were also used to survey mutant and wild type populations for variations in transpiration efficiency and to identify ecotypes having contrasting transpiration efficiencies including the parental lines that had been used by Lister and Dean (1993). The transpiration efficiencies of the members of Lister and Dean's (1993) Recombinant Inbred Line (RIL) mapping population were then determined, and linkage analyses were performed against genetic markers to determine the chromosome regions that are linked to genetic variation in transpiration efficiency, thereby identifying a locus conditioning transpiration efficiency. Complementation tests, wherein plants were transformed with a wild-type allele at this locus confirmed the functionality of the allele in determining a transpiration efficiency phenotype.

In one exemplified embodiment of the invention, there is provided a locus associated with transpiration efficiency of A. thaliana, such as, for example the ERECTA locus on A. thaliana chromosome 2, or a hybridization probe which maps to the region between about 46 cM and about 50.7 cM on chromosome 2 of A. thaliana. In further exemplified embodiments, the inventors identified additional ERECTA alleles or erecta alleles in A. thaliana, rice, sorghum, wheat and maize which are structurally related to this primary A. thaliana ERECTA or erecta allele. Based upon the large number of ERECTA/erecta alleles described herein, the present invention clearly extends to any homologs of the A. thaliana ERECTA locus from other plant species to those specifically exemplified, and particularly when those homologs are identified using the methods described herein.

Accordingly, one aspect of the invention provides a genetic marker or locus associated with the genetic variation in transpiration efficiency of a plant, wherein said locus comprises a nucleotide sequence linked genetically to an ERECTA locus in the genome of the plant. The locus or genetic marker is useful for determining transpiration efficiency of a plant.

As used herein, the terms “genetically linked” and “map to” shall be taken to refer to a sufficient genetic proximity between a linked nucleic acid comprising a gene, allele, marker or other nucleotide sequence and nucleic acid comprising all or part of an ERECTA locus to permit said linked nucleic acid to be useful for determining the presence of a particular allele of said ERECTA locus in the genome of a plant. Those skilled in the art will be aware that for such linked nucleic acid to be used in this manner, it must be sufficiently close to said locus not to be in linkage disequilibrium or to have a high recombination frequency between said linked nucleic acid and said locus. Preferably, the linked nucleic acid and the locus are less than about 25 cM apart, more preferably less than about 10 cM apart, even more preferably less than about 5 cM apart, still even more preferably less than about 3 cM apart and still even more preferably less than about 1 cM apart.

In a preferred embodiment the present invention provides an isolated nucleic acid associated with the genetic variation in transpiration efficiency of a plant, said nucleic acid comprising a nucleotide sequence selected from the group consisting of:
(a) the sequence of an ERECTA genomic gene or the 5′-UTR or 3′-UTR or protein-encoding region or an intron region thereof;
(b) the sequence of an allelic variant of (a) or the 5′-UTR or 3′-UTR or protein-encoding region or an intron region of said allelic variant;
(c) the sequence of a fragment of (a) or (b) that hybridizes specifically to nucleic acid (eg., RNA or DNA) from a plant under at least low stringency hybridization conditions; and
(d) a sequence that is complementary to (a) or (b) or (c).

In a particularly preferred embodiment, the present invention provides an isolated ERECTA gene from wheat comprising a nucleotide sequence selected from the group consisting of:

(i) the sequence set forth in SEQ ID NO: 19;

(ii) a sequence encoding the amino acid sequence set forth in SEQ ID NO: 20; and

(iii) a sequence that is complementary to (i) or (ii).

In an alternative embodiment, the present invention provides an isolated ERECTA gene from maize comprising a nucleotide sequence selected from the group consisting of:

(i) the sequence set forth in SEQ ID NO: 44;

(ii) a sequence encoding the amino acid sequence set forth in SEQ ID NO: 45; and

(iii) a sequence that is complementary to (i) or (ii).

In another alternative embodiment, the present invention provides an isolated ERECTA gene from rice comprising a nucleotide sequence selected from the group consisting of:

(i) the sequence set forth in SEQ ID NO: 3;

(ii) a sequence encoding the amino acid sequence set forth in SEQ ID NO: 4; and

(iii) a sequence that is complementary to (i) or (ii).

In another alternative embodiment, the present invention provides an isolated ERECTA gene from A. thatliana comprising a nucleotide sequence selected from the group consisting of:

(i) the sequence set forth in SEQ ID NO: 1;

(ii) a sequence encoding the amino acid sequence set forth in SEQ ID NO: 2; and

(iv) a sequence that is complementary to (i) or (ii).

In another alternative embodiment, the present invention provides an isolated ERECTA gene from A. thatliana comprising a nucleotide sequence selected from the group consisting of:

(i) the sequence set forth in SEQ ID NO: 7;

(ii) a sequence encoding the amino acid sequence set forth in SEQ ID NO: 8; and

(v) a sequence that is complementary to (i) or (ii).

In another alternative embodiment, the present invention provides an isolated ERECTA gene from A. thatliana comprising a nucleotide sequence selected from the group consisting of:

(i) the sequence set forth in SEQ ID NO: 9;

(ii) a sequence encoding the amino acid sequence set forth in SEQ ID NO: 10; and

(vi) a sequence that is complementary to (i) or (ii).

In yet another alternative embodiment, the present invention provides an isolated ERECTA gene from sorghum comprising a nucleotide sequence selected from the group consisting of:

(i) the sequence set forth in SEQ ID NO: 5;

(ii) a sequence encoding the amino acid sequence set forth in SEQ ID NO: 6; and

(vii) a sequence that is complementary to (i) or (ii).

Notwithstanding that an ERECTA or erecta structural gene or genomic gene or the protein encoding region thereof is particularly useful for breeding and/or mapping purposes, this aspect of the present invention is not to be limited to the ERECTA or erecta structural or genomic gene or the protein-encoding region thereof. As exemplified herein, the primary A. thaliana ERECTA locus can be determined using any linked nucleic acid that maps to a region in the chromosome at a genetic distance of up to about 3 cM from the ERECTA or erecta allele. The skilled artisan will readily be able to utilize similar probes to identify linkage to an ERECTA or erecta allele in any other plant species, based upon the teaching provided herein that the ERECTA or erecta allele is linked to the transpiration efficiency phenotype of plants.

Preferably, all or part of the locus associated with the transpiration efficiency phenotype in a plant (ie., nucleic acid genetically linked to the ERECTA or erecta structural or genomic gene) is provided as recombinant or isolated nucleic acid, such as, for example, in the form of a gene construct (eg. a recombinant plasmid or cosmid), to facilitate germplasm screening.

The ERECTA locus or a gene that is linked to the ERECTA locus is particularly useful in a breeding program, to predict the transpiration efficiency of a plant, or alternatively, as a selective breeding marker to select plants having enhanced transpiration efficiency. Once mapped, marker-assisted selection (MAS) is used to introduce the ERECTA locus or markers linked thereto into a wide variety of populations. MAS has the advantage of reducing the breeding population size required, and the need for continuous recurrent testing of progeny, and the time required to develop a superior line.

Accordingly, a further aspect of the present invention provides a method of selecting a plant having enhanced transpiration efficiency, comprising detecting a genetic marker for transpiration efficiency which marker comprises a nucleotide sequence linked genetically to an ERECTA locus in the genome of the plant and selecting a plant that comprises or expresses the genetic marker, preferably wherein the genetic marker comprises an ERECTA allele or erecta allele, or a protein-encoding portion thereof, or alternatively, wherein the genetic marker comprises a nucleotide sequence having at least about 55% overall sequence identity to at least about 20 nucleotides in length of any one of SEQ ID Nos: 1, 3, 5, 7, 9, 11 to 19 or 21 to 44 or a complementary sequence thereto, including a nucleotide sequence selected from the group consisting of:
(a) a sequence having at least about 55% identity to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43, SEQ ID NO: 44;
(b) a sequence encoding an amino acid sequence having at least about 55% identity to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45; and
(c) a sequence complementary to (a) or (b).

In an alternative embodiment, the invention provides a method of selecting a plant having enhanced transpiration efficiency, comprising:
(a) screening mutant or near-isogenic or recombinant inbred lines of plants to segregate alleles at an ERECTA locus;
(b) identifying a polymorphic marker linked to said ERECTA locus; and
(c) selecting a plant that comprises or expresses the marker.

The data exemplified herein for A. thaliana or rice can clearly be extrapolated to other plant species. For example, the evidence provided herein for the role of the A. thaliana ERECTA allele in determining the transpiration efficiency phenotype in those plant species has permitted the elucidation of a wide range of homologous ERECTA alleles in other plant species, in particular wheat, rice, sorghum and maize, that are also likely to determine the transpiration efficiency phenotype in those plants. In accordance with this embodiment, the present invention provides a method of selecting a plant having enhanced transpiration efficiency, comprising selecting a plant that comprises or expresses a functionally equivalent homolog of a protein-encoding region of the ERECTA gene of A. thaliana, maize, wheat, sorghum or rice.

In a preferred embodiment, the invention provides a method of selecting a plant having enhanced transpiration efficiency, comprising:
(a) identifying a locus on the Arabidopsis chromosome 2 (46-50.7 cM) or rice chromosome 6 associated with genetic variation in transpiration efficiency in a plant;
(b) identifying nucleic acid in a different plant species that comprises a nucleotide sequence having at least about 55% identity to the sequence of the locus at (a); and
(c) selecting a plant that comprises or expresses the identified nucleic acid at (b).

In a further preferred embodiment, this aspect of the invention provides a method of selecting a plant having enhanced transpiration efficiency, comprising:
(a) identifying a locus on the Arabidopsis chromosome 2 (46-50.7 cM) or rice chromosome 6 associated with genetic variation in transpiration efficiency in a plant;
(b) determining the nucleotide sequence of the identified locus;
(c) identifying nucleic acid of a plant species other than A. thaliana or rice that comprises a nucleotide sequence having at least about 55% identity to the sequence of the locus at (a); and
(d) selecting a plant that comprises or expresses the identified nucleic acid at (b).

Preferably, the selected plant according to any one or more of the preceding embodiments is Arabidopsis thaliana, rice, sorghum, wheat or maize, however other species are not excluded.

Preferably, the subject selection method comprises linking the transpiration efficiency phenotype of the plant to the expression of the marker in the plant, or alternatively, linking a structural polymorphism in DNA to a transpiration efficiency phenotype in the plant, eg., by a process comprising detecting a restriction fragment length polymorphism (RFLP), amplified fragment length polymorphism (AFLP), single strand chain polymorphism (SSCP) or microsatellite analysis. As will be known to the skilled artisan, a nucleic acid probe or primer of at least about 20 nucleotides in length from any one of SEQ ID Nos: 1, 3, 5, 7, 9, 11 to 19 or 21 to 44 or a complementary sequence thereto can be hybridized to genomic DNA from the plant, and the hybridization detected using a detection means, thereby identifying the polymorphism.

It is clearly preferred that the selected plant has enhanced transpiration efficiency compared to a near-isogenic plant that does not comprise or express the genetic marker.

As exemplified herein, the inventors also identified specific genes or alleles that are linked to the ERECTA locus of A. thaliana, and rice and determined the transpiration efficiencies of those plants. More particularly, the transpiration efficiencies of near-isogenic lines, each carrying a mutation within an ERECTA locus, and a correlation between transpiration efficiency phenotype and ERECTA expression or gene copy number are determined, thereby providing the genetic contribution of genes or alleles at the ERECTA locus to transpiration efficiency. This analysis permits an assessment of the genetic contribution of particular alleles to transpiration efficiency, thereby determining allelic variants that are linked to a particular transpiration efficiency. Thus, the elucidation of the ERECTA locus for transpiration efficiency in plants facilitates the fine mapping and determination of allelic variants that modulate transpiration efficiency. The methods described herein can be applied to an assessment of the contribution of specific alleles to the transpiration efficiency phenotype for any plant species that is amenable to mutagenesis such as, for example, by transposon mutagenesis, irradiation, or chemical means. As will be known to the skilled artisan many crop species, such as, maize, wheat, and rice, are amenable to such mutagenesis.

Accordingly, a third aspect of the invention provides a method of identifying a gene that determines the transpiration efficiency of a plant comprising:
(a) identifying a locus associated with genetic variation in transpiration efficiency in a plant;
(b) identifying a gene or allele that is linked to said locus, wherein said gene or allele is a candidate gene or allele for determining the transpiration efficiency of a plant; and
(c) determining the transpiration efficiencies of a panel of plants, wherein not all members of said panel comprise or express said gene or allele, and wherein variation in transpiration efficiency between the members of said panel indicates that said gene is involved in determining transpiration efficiency.

In another embodiment, the method comprises:
(a) identifying a locus associated with genetic variation in transpiration efficiency in a plant;
(b) identifying multiple alleles of a gene that is linked to said locus, wherein said gene is a candidate gene involved for determining the transpiration efficiency of a plant; and
(c) determining the transpiration efficiencies of a panel of plants, wherein each member of said panel comprises, and preferably expresses, at least one of said multiple alleles, wherein variation in transpiration efficiency between the members of said panel indicates that said gene is involved in determining transpiration efficiency.

Preferably, the identified gene or allele identified by the method described in the preceding paragraph is an ERECTA allele, or an erecta allele, from a plant selected from the group consisting of A. thaliana, sorghum, rice, maize and wheat, or a homolog thereof.

The identified gene or allele, including any homologs from a plant other than A. thaliana, such as, for example, the wild-type ERECTA allele or a homolog thereof, is useful for the production of novel plants. Such plants are produced, for example, using recombinant techniques, or traditional plant breeding approaches such as introgression.

Accordingly, a still further aspect of the present invention provides a method of modulating (i.e., enhancing or reducing) the transpiration efficiency of a plant comprising ectopically expressing in a plant an isolated ERECTA gene or an alleic variant thereof or the protein-encoding region of said ERECTA gene or said allelic variant. In a particularly preferred embodiment, the invention provides a method of enhancing the transpiration efficiency of a plant comprising introgressing into said plant a nucleic acid comprising a nucleotide sequence that is homologous to a protein-encoding region of a gene of A. thaliana that maps to the ERECTA locus on chromosome 2.

A further embodiment of the invention provides a method of modulating the transpiration efficiency of a plant comprising introducing (eg., by classical breeding, introgression or recombinant means), and preferably expressing therein, an isolated ERECTA gene or an allelic variant thereof or the protein-encoding region thereof to a plant and selecting a plant having a different transpiration efficiency compared to a near-isogenic plant that does not comprise the introduced ERECTA gene or allelic variant or protein-encoding region. Preferably, the ERECTA gene or allelic variant or protein-encoding region comprises a nucleotide sequence selected from the group consisting of:
(a) a sequence having at least about 55% identity to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44; and
(b) a sequence encoding an amino acid sequence having at least about 55% identity to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45.

The plant into which the gene etc is introduced is preferably selected from the group consisting of Arabidopsis thaliana, rice, sorghum, wheat and maize. As will be apparent from the present disclosure, the transpiration efficiency is enhanced as a consequence of the ectopic expression of an ERECTA allele or the protein-encoding region thereof in the plant. In contrast, the transpiration efficiency is reduced as a consequence of reduced expression of an ERECTA allele in the plant (eg., by expression of antisense RNA or RNAi or other inhibitory RNA).

A further aspect of the invention provides for the use of an isolated ERECTA gene or an allelic variant thereof or the protein-encoding region of said ERECTA gene or said allelic variant in the preparation of a gene construct for modulating (ie., enhancing or reducing) the transpiration efficiency of a plant. For example, expression of ERECTA protein in the plant can be modified by ectopic expression of an ERECTA allele in the plant, or alternatively, by reducing endogenous ERECTA expression using an inhibitory RNA (eg, antisense or RNAi).

A fifth aspect of the present invention provides a plant having enhanced transpiration efficiency, wherein said plant is produced by a method described herein.

Plants that have enhanced transpiration efficiency show increased levels of growth under normal growth conditions, thereby increasing their biomass. Accordingly, a further aspect of the present invention provides a method of increasing the biomass of a plant comprising enhancing the level of expression of an ERECTA gene or allelic variant thereof or protein coding region thereof in said plant.

In one embodiment, the method further includes the step of selecting a plant that has an increased biomass when compared to an unmodified plant. Methods of determining the biomass of a plant are well known to those skilled in the art and/or described herein.

In one embodiment, the level of expression is enhanced by genetic modification of a control sequence, for example a promoter sequence, associated with the ERECTA gene or allelic variant thereof.

In another embodiment, the level of expression is enhanced by introducing (eg., by classical breeding, introgression or recombinant means) an ERECTA gene or allelic variant thereof or the protein encoding region thereof to a plant. Preferably, the ERECTA gene or allelic variant or protein-encoding region comprises a nucleotide sequence selected from the group consisting of:

a sequence having at least about 55% identity to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44; and

a sequence encoding an amino acid sequence having at least about 55% identity to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45.

The plant into which the gene etc is introduced is preferably selected from the group consisting of Arabidopsis thaliana, rice, sorghum, wheat and maize.

A further aspect of the present invention provides a method of increasing the resistance of a plant to an environmental stress comprising enhancing the level of expression of an ERECTA gene or allelic variant thereof or protein coding region thereof in said plant.

As used herein the term “environmental stress” shall be taken in its broadest context to mean one or more environmental conditions that reduce the ability of a plant to grow, survive and/or produce seed/grain. In one embodiment, an environmental stress that affects the ability for a plant to grow, survive and/or produce seed/grain is a condition selected from the group consisting of increased or decreased CO₂ levels, increased or decreased temperature, increased or decreased rainfall, increased or decreased humidity, increased salt levels in the soil, increased soil strength and compaction and drought.

In one embodiment, the method further includes the step of selecting a plant that has an altered resistance to an environmental stress when compared to an unmodified plant is selected. Methods of determining the resistance of a plant to environmental stress are well known to those skilled in the art and/or described herein.

In one embodiment, the level of expression is enhanced by genetic modification of a control sequence, for example a promoter sequence, associated with the ERECTA gene or allelic variant thereof.

In another embodiment, the level of expression is enhanced by introducing (eg., by classical breeding, introgression or recombinant means) an ERECTA gene or allelic variant thereof or the protein encoding region thereof to a plant. Preferably, the ERECTA gene or allelic variant or protein-encoding region comprises a nucleotide sequence selected from the group consisting of:

a sequence having at least about 55% identity to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44; and

a sequence encoding an amino acid sequence having at least about 55% identity to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45.

The plant into which the gene etc is introduced is preferably selected from the group consisting of Arabidopsis thaliana, rice, sorghum, wheat and maize.

A further aspect of the present invention provides a plant having increased resistance to environmental stress, wherein said plant is produced by a method described herein.

Both temperature and available moisture have been shown to dramatically influence pollination and grain/seed development, processes known as seed-set and grain-filling. Accordingly, a method that produces a plant that is resistant to environmental stress, ie a plant that has increased transpiration efficiency, results in increased or more efficient grain-filling and greater seed number. As ERECTA is expressed during flowering or pod development this gene or an allelic variant thereof is useful for increasing grain-filling in a plant.

Accordingly, a further aspect of the present invention provides a method of increasing seed or grain weight in a plant comprising enhancing the level of expression of an ERECTA gene or allelic variant thereof or protein coding region thereof in said plant.

In one embodiment, the method further includes the step of selecting a plant that has increased seed or grain weight when compared to an unmodified plant is selected. Methods of determining seed or grain weight are well known to those skilled in the art and/or described herein.

In one embodiment, the level of expression is enhanced by genetic modification of a control sequence, for example a promoter sequence, associated with the ERECTA gene or allelic variant thereof.

In another embodiment, the level of expression is enhanced by introducing (eg., by classical breeding, introgression or recombinant means) an ERECTA gene or allelic variant thereof or the protein encoding region thereof to a plant. Preferably, the ERECTA gene or allelic variant or protein-encoding region comprises a nucleotide sequence selected from the group consisting of:

a sequence having at least about 55% identity to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44; and

a sequence encoding an amino acid sequence having at least about 55% identity to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45.

The plant into which the gene etc is introduced is preferably selected from the group consisting of Arabidopsis thaliana, rice, sorghum, wheat and maize.

A further aspect of the present invention provides a plant having increased seed or grain weight, wherein said plant is produced by a method described herein.

A still further aspect of the present invention provides a method of modulating the number of seeds produced by a plant comprising enhancing the level of expression of an ERECTA gene or allelic variant thereof in said plant.

In one embodiment, the method further includes the step of selecting a plant that has an increased number of seeds when compared to an unmodified plant is selected. Methods of determining seed or grain number are well known to those skilled in the art and/or described herein.

In one embodiment, the level of expression is enhanced by genetic modification of a control sequence, for example a promoter sequence, associated with the ERECTA gene or allelic variant thereof.

In another embodiment, the level of expression is enhanced by introducing (eg., by classical breeding, introgression or recombinant means) an ERECTA gene or allelic variant thereof or the protein encoding region thereof to a plant. Preferably, the ERECTA gene or allelic variant or protein-encoding region comprises a nucleotide sequence selected from the group consisting of:

a sequence having at least about 55% identity to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44; and

a sequence encoding an amino acid sequence having at least about 55% identity to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45.

The plant into which the gene etc is introduced is preferably selected from the group consisting of Arabidopsis thaliana, rice, sorghum, wheat and maize.

A further aspect of the present invention provides a plant having an increased number of seeds, wherein said plant is produced by a method described herein.

### BRIEF DESCRIPTION OF THE DRAWINGS

FIG. 1a is a graphical representation showing the CO₂ assimilation rates (μmol C m² s⁻¹) of several genotypes of A. thaliana. Measurements were completed on rosette leaves during bolting and flowering stages. Plants were grown on fertilised soil. The genotypes of plants are indicated on the x-axis, and CO₂ assimilation rates indicated on the ordinate. Col indicates a genetic background of the ecotype Columbia. Ld indicates a genetic background of the ecotype Landsberg. Plants expressing wild type ERECTA alleles were either in a Col (Col4-ER) or Ld (Ld-ER) background. Plants that were homozygous for a mutant er allele were either in a Ld background (Ld-er1) or in a Col background (Col-er105 or Col-er2 (line 3401 at NASC, also named Col-er106 by Torii and collaborators (see Lease et al. 2001, New Phytologist, 151:133-143)). Plants designated as F1 (Col-ER x Ld-er) were heterozygous ER/er1. Data indicate that, in a Col background, the er105 mutation leads to reduced CO₂ assimilation rate, whilst the er1 mutation enhances CO₂ assimilation rate in a Ld background.

FIG. 1b is a graphical representation showing the stomatal conductance (mol H₂0 m² s⁻¹) of several genotypes of A. thaliana (same plants as FIG. 1a). The genotypes of plants are indicated on the x-axis and are the same as described in the legend to FIG. 1a. Stomatal conductances are indicated on the ordinate. Data indicate that, in a Col background, the er2/er106 mutation significantly enhances stomatal conductance, whilst the er1 mutation significantly enhances stomatal conductance in a Ld background.

FIG. 1c is a graphical representation showing the transpiration efficiency of (mmol C mol H₂0⁻¹) of several genotypes of A. thaliana, as determined by the ratio of CO₂ assimilation rate to stomatal conductance. The genotypes of plants are indicated on the x-axis and are the same as described in the legend to FIG. 1a. Transpiration efficiency is indicated on the ordinate. Data indicate that transpiration efficiency is enhanced in plants expressing a wild type ER allele relative to a mutant er allele, in both Ld and Col backgrounds. The lowest transpiration efficiency was observed for plants that are homozygous for the er105 allele (ie. Col-er105), consistent with the fact that this allele inhibits ERECTA expression. From the data in FIGS. 1a-1c, it is apparent that the lower transpiration efficiency of plants expressing the er105 allele is largely due to a reduced CO₂ fixation rate, whereas for both the er2/er106 and er1 alleles, reduced transpiration efficiency is largely due to enhanced stomatal conductance. The transpiration efficiency of the F1 heterozygote plant was intermediate between the transpiration efficiencies of its parents, suggesting codominance of these alleles. The F1, however, had a transpiration efficiency closer to that of the pollen donor parent, Ld-er1.

FIG. 2a is a graphical representation showing the stomatal densities (Number of stomata mm⁻² leaf) for several genotypes of A. thaliana in three independent experiments. The genetic backgrounds of plants are indicated on the x-axis (Col, Columbia; Ld, Landsberg), and stomatal densities are indicated on the ordinate. Plant genotypes are indicated at the top of each bar, as follows: plants expressing wild type ERECTA alleles in a Col background were Col4ER or Col1ER (hatched bars); plants expressing wild type ERECTA alleles in a Ld background were ER (open bars); plants expressing mutant erecta alleles in a Col background were either er105 or er2/106 (Col filled boxes); and plants expressing the mutant er1 allele in a Ld background were er1 (Ld filled boxes). Columns designated a,b are data from two experiments where plants were grown in soil in the absence of fertiliser. The set of columns at the right of the figure are from a third experiment where the same plants were grown in soil comprising fertiliser. Data indicate that, in a Col background, the er105 mutation and er2/106 mutation enhances stomatal density, which in part accounts for the enhanced stomatal conductances and reduced transpiration efficiencies of plants expressing these alleles (FIGS. 1b and 1c). The general effect of these alleles is not dependent on the nutrient status of the soil. In contrast, the er1 allele only enhanced stomatal density of Ld plants when fertiliser was absent, suggesting that in this ecotype enhanced stomatal aperture accounted for the enhanced stomatal conductances and reduced transpiration efficiencies measured in the er1 mutant under ample nutrient supply (FIGS. 1b, 1c). The er1 mutation therefore affects both stomatal aperture and stomatal density but the relative contributions of these effects to enhanced stomatal conductance per unit leaf area depend on environmemtal factors and plant nutrient status, and on genetic background.

FIG. 2b is a graphical representation showing the epidermal cell size (surface area, μm²) for several genotypes of A. thaliana in three independent experiments. The genetic backgrounds and genotypes of plants are indicated on the x-axis and at the tops of each column, respectively, as in the legend to FIG. 2a. The ordinate indicates epidermal cell size. Columns designated a,b are data from two experiments where plants were grown in soil in the absence of fertiliser. The set of columns at the right of the figure are from a third experiment where the same plants were grown in soil comprising fertiliser. Data indicate that, in a Col background, the er105 mutation and er2/er106 mutation significantly reduce epidermal cell size ie increase the number of epidermal cells per unit leaf area. This reveals that the ER gene has effects on leaf histogenesis which, beyond their consequences on stomatal densities, may also directly affect leaf capacity for photosynthesis and therefore transpiration efficiency, (FIGS. 1b and 1c). The general effects of these alleles are not dependent on the nutrient status of the soil. In contrast, in a Ld background, the er1 allele reduced epidermal cell size only when fertiliser was absent.

FIG. 2c is a graphical representation showing the stomatal index for several genotypes of A. thaliana in three independent experiments. The genetic backgrounds and genotypes of plants are indicated on the x-axis and at the tops of each column, respectively, as in the legend to FIG. 2a. The ordinate indicates stomatal index, as determined from the ratio of stomatal density to epidermal cell density. Columns designated a,b are data from two experiments where plants were grown in soil in the absence of fertiliser. The set of columns at the right of the figure are from a third experiment where the same plants were grown in soil comprising fertiliser. Data indicate that the er mutations tested do not significantly modify stomatal index in Col background (because increases in stomatal density are correlated to increases in epidermal cell numbers in the Col mutant plants) but does so in Landsberg background. Accordingly, the ER gene does appear to directly modify stomatal development per se. Taken together FIGS. 2a-c therefore show that the ERECTA gene has two types of effects on leaf stomatal conductance: a) developmental, b) biophysical and/or biochemical. The expression of these effects and impact on transpiration rate vary with genetic background, suggesting interactions with other genes that are polymorphic between the Col and Ld ecotypes, and also with nutrient status.

FIG. 3 is a graphical representation showing carbon isotope composition (y-axis; in per mil, for vegetative rosettes) for 7 different experimental runs (numbers 1-7) carried out under growth cabinet conditions and glasshouse conditions. For each run, the left-hand side bar shows the mean value of carbon isotope composition for lines carrying the ERECTA allele, while the right-hand side bar shows the mean value across lines with the erecta allele. In all cases, δ¹³C isotopic composition values for the er-lines are more negative then those for ER lines, indicative of lower transpiration efficiencies.

FIG. 4a is a graphical representation showing ERECTA gene copy number and expression levels in transgenic T2 A. thaliana plants homozygous for an ER transgene. These lines were generated by transforming the Col-er2/106 mutant with the wild type ER gene under the 35S promoter. Effective transformation was ascertained and ERECTA expression levels were quantified in several independent transformants using real-time quantitative PCR (ABI PRISM 7700, Sequence Detection System User Bulletin #2. 1997). Copy number (y-axis) is indicated as a function of the plant line, following normalisation of ERECTA relative to the copy number of a control gene (18S ribosomal RNA gene). The expression of the 18S rRNA gene was shown independently not to be affected by changes in ER expression. Line 143 is null control (no insert). Lines 145, 165, 169 and 279 are transformed lines carrying the ERECTA allele. All ER transgenic lines, except line 145, show increased mRNA copy number: from 4 to 9.5 fold increase compared with the null control.

FIG. 4b is a graphical representation showing ERECTA gene copy number and expression levels in transgenic T2 A. thaliana plants homozygous for an ER transgene, and generated by transformation of the Col-er105 mutant. Effective transformation was ascertained and ERECTA expression levels were quantified in several independent transformants using real-time quantitative PCR (ABI PRISM 7700, Sequence Detection System User Bulletin #2. 1997). Copy number (y-axis) is indicated as a function of the plant line, following normalisation of ERECTA relative to the copy number of a control gene (18S ribosomal RNA gene). The expression of the 18S rRNA gene was shown independently not to be affected by changes in ER expression. Line 18 is a null control line (no ER insert, ie similar to Col-er105). Lines 8, 19, 29 and 61 are transgenic lines carrying the ERECTA allele. All ER transgenic lines show increased mRNA copy number: from 10 to 170 fold increase compared with the null control.

FIG. 4c is a graphical representation showing ERECTA gene copy number and expression levels in Col and Ld ER ecotypes and in one Ld-ER transgenic line (3-7K) generated by transformation of the Ld-er1 ecotype (NW20) with the ER wild type gene under control of the 35S promoter. Effective transformation was ascertained and ERECTA expression levels were quantified in several independent transformants using real-time quantitative PCR (ABI PRISM 7700, Sequence Detection System User Bulletin #2. 1997). Copy number (y-axis) is indicated as a function of the plant line, following normalisation of ERECTA relative to the copy number of a control gene (18S ribosomal RNA gene). The expression of the 18S rRNA gene was shown independently not to be affected by changes in ER expression. Lines 933, 1093 and 3176 are the non-transformed Columbia-ERECTA ecoptypes Col-4, Col-0 and Col-1. Line 105c is a Col-er105 line (knockout for ER), used for generating transgenic lines shown in FIG. 4b. Lines labelled 2c and 3401 on the X-axis describe Col-er2/106 (2 batches of seeds, used for generating transgenic lines shown in FIG. 4a). Line NW20 is Ld-er1. Line 3-7K is a Ld-ER transformant, obtained from transformation of Ld-er1 with the ERECTA allele. Line 3177 is the Ld-ER ecotype, near-isogenic to NW20.

FIG. 5a is a graphical representation of a first experiment showing copy number of the mRNA transcription product of the rice ERECTA gene in various plant organs/parts, cv Nipponbare. L=mature leaf blades; YL=young expanding leaves, still enclosed in sheaths of older leaves; R=mature root; YR=young root; SH=sheaths; INF=unfolded young panicle still enclosed in sheaths; O7: young panicles. Rice ERECTA mRNA copy numbers were determined by quantitative real-time PCR, with 18S mRNA as internal control gene for normalization of results. The values on the y-axis describe fold increases of rice ERECTA mRNA in various parts compared to the L sample (mature leaves) set to a value of 1 for normalization. Data show a similar expression pattern as the ERECTA gene in Arabidopsis (see Torii et al. 1996) ie preferential expression in young meristematic tissues, especially in reproductive organs.

FIG. 5b is a graphical representation of a second experiment showing copy number of the mRNA transcription product of the rice ERECTA gene in various plant organs/parts. L=mature leaf blades; YL=young expanding leaves, still enclosed in sheaths of older leaves; R=mature root; YR=young root; SH=sheaths; INF=unfolded young panicle still enclosed in sheaths; 07: young panicles. Rice ERECTA mRNA copy numbers were determined by quantitative real-time PCR, with 18S mRNA as internal control gene for normalization of results. The values on the y-axis describe fold increases of rice ERECTA mRNA in various parts compared to the L sample (mature leaves) set to a value of 1 for normalization. Data confirm those shown in FIG. 5a.

FIG. 6 is a graphical representation showing leaf transpiration efficiency (mmol C mol H₂O⁻¹, FIG. 6a), calculated from the direct measurements of leaf CO₂ assimilation rate (μmol C m⁻² s⁻¹, FIG. 6b) and stomatal conductance (mol H₂O m⁻² s⁻¹, FIG. 6c) by gas exchange techniques, under 350 ppm CO₂ (ie same as ambient [CO₂] during seedling growth; left hand bar in each pair of bars) and 500 ppm CO₂ (right hand bar in each pair of bars), for Ld-er1, and two Ld_ER lines: line T2(+ER), a T2 transgenic line homozygous for an ER transgene in the Ld-er1 background and line 3177, an ER ecotype near-isogenic to Ld-er1 (NASC Stock Centre information). Genotypes are shown at the bottom of the figure. Leaf temperature during measurements was controlled at 22° C., leaf to air vapour pressure deficit at around 8 mb.

FIG. 7 is a graphical representation showing leaf transpiration efficiency (mmol C mol H₂O⁻¹, FIG. 7a), calculated from the direct measurements of leaf CO₂ assimilation rate (μmol C m⁻² s⁻¹, FIG. 7b) and stomatal conductance (mol H₂O m⁻² s⁻¹, FIG. 7c) by gas exchange techniques, under 350 ppm CO₂ (ie same as ambient [CO₂] during seedling growth; left hand bar in each pair of bars) and 500ppm CO₂ (right hand bar in each pair of bars), for 4 genotypes: Col4 (ER) (left hand pair), Ld (er1) (right hand pair) and their F₁ progeny (middle two pairs). Genotypes are shown at the bottom of the figure.

FIG. 8 is a graphical representation showing stomatal conductance and epidermal anatomy at 350 ppm CO₂ in the genotypes described in FIGS. 6 and 7 and shown at the bottom of the figure. The insertion of ER transgene (line T2+ER) caused a decreased in stomatal conductance compared to the Ld-er1 line (FIG. 8a), which was in part due to a decrease in stomatal density (see FIG. 8c). These two effects again indicate complementation. Together FIGS. 8b and 8c show that the decrease in stomatal density is relatively more important than that in epidermal cell density, indicating an effect of the transgene on epidermis development.

FIG. 9 is a graphical representation showing a comparison of stomatal density and epidermal cell area in a range of Col er lines carrying mutations in the ER gene (bars 1 to 8 FIG. 9a; bar 1 to 7 in FIG. 9b, mutants er105, er106, 108, 111, 114, 116, 117, as described in Lease et al. 2001; a gift from Dr Keiko Torii) and in Col-ER wild type ecotypes (bars 9-11 or 8-10 in FIGS. 9a and 9b, respectively: Col0, background ecotype for these mutants; Col1, Col4 (ColER parental line for QTL analysis of Lister and Dean's RILs), two Ld_er1 lines (NW20 and CS20, bars 12&13 and 11&12 in FIGS. 9a and 9b respectively, two very similar lines according to NASC; NW20 is the other parental line for Lister and Dean's RILs) and finally line T2+ER, a transgenic Ld-ER line carrying the ER wild type gene in Ld-er1 background (extreme right hand bar on the figure).

FIG. 10 is a graphical representation showing carbon isotopic composition (per mil, y-axis) in a range of lines (numbered 1 to 19 on the x-axis): Col-er mutants (line 1-14); the Col0 background ecotype (line 15); Ld-er1 lines (lines 16 and 17); an Ld-ER near isogenic ecotype to Ld-er1 (line 18, line 3177 at NASC), and a transgenic T2 Ld-ER line (line numbered 19) obtained by transformation of Ld-er1 mutant with a construct carrying the wild type ER allele. The data show that the ER allele gives less negative values indicative of increased transpiration efficiency.

FIG. 11 is a graphical representation showing direct measurements of transpiration efficiency in Col-er mutants transformed with ER transgene, under both high and low air humidity, such as occurs during hot temperature events causing or associated with drought. Transpiration efficiency was measured by gas exchange techniques on mature leaves of vegetative Arabidopsis rosettes, as a function of leaf-to-air vapour pressure difference (vpd) ie air humidity around the leaves. The higher the vpd, the drier the air. Solid circles describe measurements for 5 independent transgenic T2 lines homozygous for an ER transgene; these lines were generated by transforming the Col-er105 mutant (empty squares) with a construct carrying the ER allele under control of the 35S promoter. Data for null lines (ie lines that went through transgenesis but do not carry the ER transgene) are represented by solid squares. This figure demonstrates complementation, across the whole range of humidity tested, with the transpiration efficiencies in T2 ER lines being greater than those in the complemented Col-er105 mutant, and similar to those measured in the Col0-ER ecotype (empty triangles; background ecotype for Col-er105).

FIG. 12 is a graphical representation of an alignment of isolated sequences with the entire coding region of the wheat ortholog of ERECTA. The position of each of the isolated sequences is shown relative to the wheat ortholog of ERECTA. Sequences are represented by either SEQ ID NO. or gene accession number.

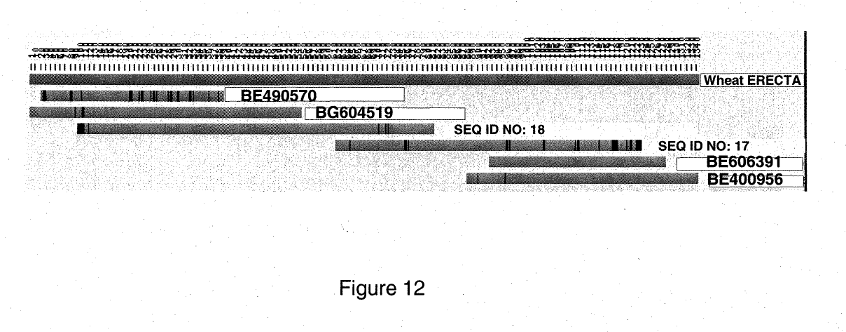

FIG. 13 is a graphical representation of an alignment of isolated sequences with the entire coding region of the maize ortholog of ERECTA. The position of each of the isolated sequences is shown relative to the maize ortholog of ERECTA. Sequences are represented by either SEQ ID NO. or gene accession number.

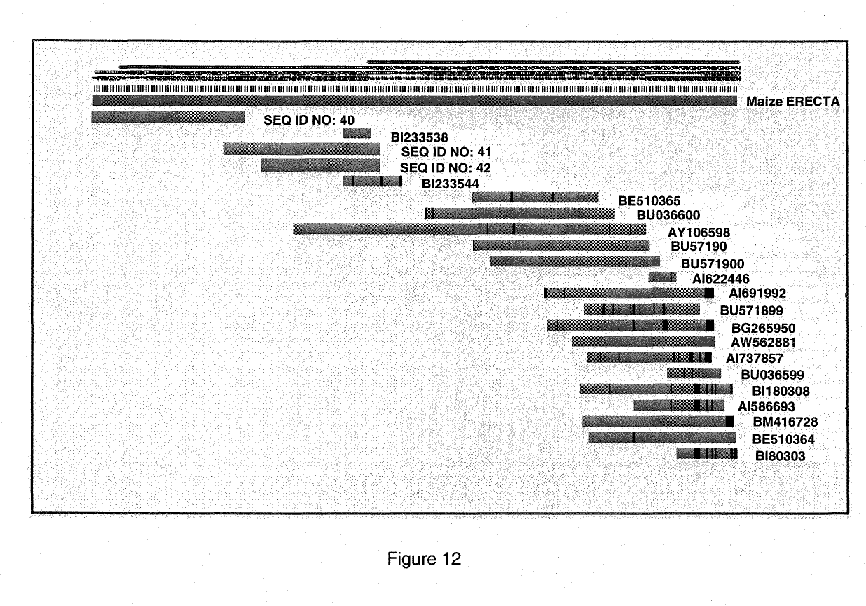

FIG. 14 is a graphical representation of a pairwise sequence alignment of the ERECTA proteins isolated from Arabidopsis (SEQ ID NO: 2), maize (SEQ ID NO: 45), rice (SEQ ID NO: 3), Sorghum (SEQ ID NO: 5) and wheat (SEQ ID NO: 20). The alignment was performed using CLUSTALW multiple sequence alignment tool. Residues that are conserved between all species are indicated by asterisks (*). Conservation of the groups STA NEQK NHQK NDEQ QHRK MILV MILF HY or FYW is indicated by “:”. Conservation of the groups CSA ATV SAG STNK STPA SGND SNDEQK NDEQHK NEQHRK FVLIM HFY is indicated by “.”. Gaps are indicated by dashes “-”.

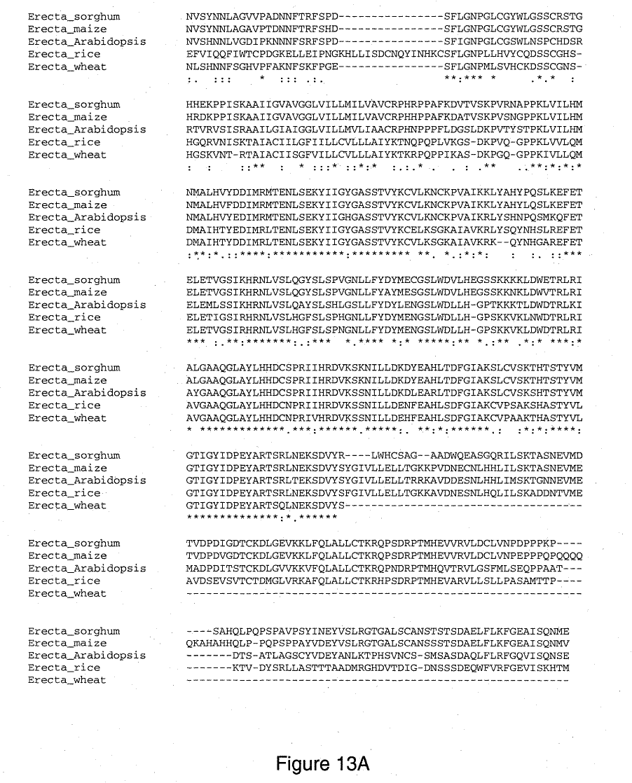

FIG. 15 is a graphical representation of a phylogenetic tree indicating the relationship between each of the ERECTA proteins isolated from Arabidopsis (SEQ ID NO: 2), maize (SEQ ID NO: 45), rice (SEQ ID NO: 3), Sorghum (SEQ ID NO: 5) and wheat (SEQ ID NO: 20).

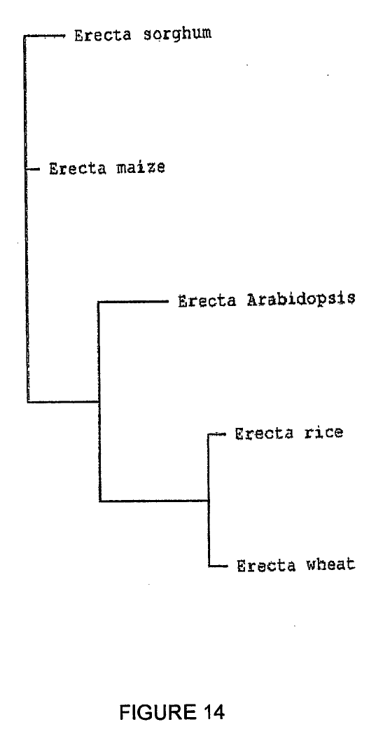

### DETAILED DESCRIPTION OF THE INVENTION

Loci for Transpiration Efficiency and their Identification

One aspect of the invention provides a locus associated with the genetic variation in transpiration efficiency of a plant, wherein said locus comprises a nucleotide sequence linked genetically to an ERECTA locus in the genome of the plant.

As used herein, the term “locus” shall be taken to mean the location of one or more genes in the genome of a plant that affects a quantitative characteristic of the plant, in particular the transpiration efficiency of a plant. In the present context, a “quantitative characteristic” is a phenotype of the plant for which the phenotypic variation among different genotypes is continuous and cannot be separated into discrete classes, irrespective of the number of genes that determine or control the phenotype, or the magnitude of genetic effects that single gene has in determining the phenotype, or the magnitude of genetic effects of interacting genes.

By “associated with the genetic variation in transpiration efficiency of a plant” means that a locus comprises one or more genes that are expressed to determine or regulate the transpiration efficiency of a plant, irrespective of the actual rate of transpiration achieved by the plant under a specified environmental condition.

Preferably, the locus of the invention is linked to or comprises an ERECTA allele or erecta allele, or a protein-encoding portion thereof.

As used herein, the term “ERECTA” shall be taken to refer to a wild type allele comprising the following domains GTIGYIDPEYARTS, GAAQGLAYLHHDC, and TENLSEKXIIGYGASSTVYKC domains, wherein X means Y or H, or domains more than 94% identical to these domains. To the inventors' knowledge no other protein comprises these domains. Preferred ERECTA alleles comprise a nucleotide sequence having at least about 55% overall sequence identity to the protein-encoding region of any one of the exemplified ERECTA alleles described herein, particularly any one of SEQ ID Nos: 1, 3, 5, 7, 9, 11, 13, or 15. Preferably, the percentage identity to any one of said SEQ ID NOs: is at least about 59-61%, or 70% or 80%, and more preferably at least about 90%, and still more preferably at least about 95% or 99%.

Preferred ERECTA alleles are derived from, or present in, the genome of a plant that is desiccation or drought intolerant, or poorly adapted for growth in dry or arid environments, or that suffers from reduced vigor or growth during periods of reduced rainfall or drought, or from the genome of a plant with increased growth rate or growth duration or partitioning of C to shoot and harvested parts under well-watered conditions.

More preferably, an ERECTA allele is derived from, or present in, the genome of a brassica plant, broad acre crop plant, perennial grass (eg. of the subfamily Pooidaea, or the Tribe Poeae), or tree. Even more preferably, an ERECTA allele is present in or derived from the genome of a plant selected from the group consisting of barley, wheat, rye, sorghum, rice, maize, Phalaris aquatica, Dactylus glomerata, Lolium perenne, Festuca arundinacea, cotton, tomato, soybean, oilseed rape, poplar, and pine.

The term “erecta” shall be taken to mean any allelic variant of the wild-type ERECTA allele that modifies transpiration efficiency of a plant.

Preferred erecta alleles include the following A. thaliana erecta alleles derived from Columbia (Col) and Landsberg erecta (er) lines.

| Erecta alleles¹ | Genomic position | Lesion | Affected domain |
|---|---|---|---|
| Ler er-1 | 2249 | T→ A | PK |
| Col er-101 | 6565 | T→ A | PK |
| Col er-102/106 | 6565 | T→ A | PK |
| Col er-103 | 846 | G→ A | LRR10 |
| Col er-105 | foreign DNA insert | insertion | Null allele |
| | between +5 and +1056 | | |
| Col er-108 | 5649 | G→ A | |
| Col er-111 | 5749 | G→ A | Untranslated region |
| | | | between LRR and |
| | | | transmembrane |
| | | | domains |
| Col er-113 | 3274 | C→ T | |
| Col er-114 | 6807 | G→ A | PK |
| Col er-115 | 3796 | C→ T | |
| Col er-116 | 6974 | G→ A | PK |
| Col er-117 | 5203 | G→ A | LRR18 |

¹alleles described by Lease et al. 2001, New Phytologist, 151: 133-143, except for Ler er-1, Col er-103 and Col-er105 which were described in Torii et al., 1996, The Plant Cell 8: 735-746

The present invention clearly encompasses an erecta allele derived from, or present in, the genome of a plant that is desiccation or drought intolerant, or poorly adapted for growth in dry or arid environments, or that suffers from reduced vigor or growth during periods of reduced rainfall or drought, or from the genome of a plant with increased growth rate or growth duration or partitioning of C to shoot and harvested parts under well-watered conditions.

More preferably, an erecta allele is derived from, or present in, the genome of a brassica plant, broad acre crop plant, perennial grass (eg. of the subfamily Pooidaea, or the Tribe Poeae), or tree. Even more preferably, an erecta allele is present in or derived from the genome of a plant selected from the group consisting of barley, wheat, rye, sorghum, rice, maize, Phalaris aquatica, Dactylus glomerata, Lolium perenne, Festuca arundinacea, cotton, tomato, soybean, oilseed rape, poplar, and pine.

For the purposes of nomenclature, the nucleotide sequence of the Arabidopsis thaliana ERECTA protein-encoding region and the 5′-untranslated region (UTR) and 3′-UTR, is provided herein as SEQ ID NO: 1. The amino acid sequence of the polypeptide encoded by SEQ ID NO: 1 is set forth herein as SEQ ID NO: 2.

A particularly preferred ERECTA allele from rice (Oryza sativa) is derived from chromosome 6 of that plant species. For the purposes of nomenclature, the protein-encoding region of the rice ERECTA gene is provided herein as SEQ ID NO: 3. The amino acid sequence of the polypeptide encoded by SEQ ID NO: 3 is set forth herein as SEQ ID NO: 4.

A particularly preferred ERECTA gene derived from the genome of Sorghum bicolor, is provided herein as SEQ ID NO: 5. The amino acid sequence of the polypeptide encoded by SEQ ID NO: 5 is set forth herein as SEQ ID NO: 6.

A further exemplary ERECTA gene derived from A. thaliana is provided herein as SEQ ID NO: 7. The amino acid sequence of the polypeptide encoded by SEQ ID NO: 7 is set forth herein as SEQ ID NO: 8.

A further exemplary ERECTA gene derived from A. thaliana is provided herein as SEQ ID NO: 9. The amino acid sequence of the polypeptide encoded by SEQ ID NO: 9 is set forth herein as SEQ ID NO: 10.

Fragments of an exemplary ERECTA gene derived from the genome of wheat are provided herein as SEQ ID NOs: 11 to 18.

An exemplary ERECTA gene derived from the genome of wheat is provided herein as SEQ ID NO: 19. The amino acid sequence of the polypeptide encoded by SEQ ID NO: 19 is set forth herein as SEQ ID NO: 20.

Fragments of an exemplary ERECTA gene derived from the genome of maize are provided herein as SEQ ID NOs: 21 to 43.

An exemplary ERECTA gene derived from the genome of maize is provided herein as SEQ ID NO: 44. The amino acid sequence of the polypeptide encoded by SEQ ID NO: 44 is set forth herein as SEQ ID NO: 44.

The present invention clearly contemplates the presence of multiple genes that are genetically linked or map to the specified ERECTA locus on chromosome 2. Without being bound by any theory or mode of action, such multiple linked genes may interact, such as, for example, by epistatic interaction, to determine the transpiration efficiency phenotype.

The present invention also contemplates the presence of different alleles of any gene that is linked to the ERECTA locus, wherein said allele is expressed to determine the transpiration efficiency phenotype. In one embodiment, such alleles are identified by detecting a particular transpiration efficiency phenotype that is linked to the expression of the particular allele. Alternatively, or in addition, the different alleles linked to a locus are identified by detecting a structural polymorphism in DNA (eg. a restriction fragment length polymorphism (RFLP), amplified fragment length polymorphism (AFLP), single strand chain polymorphism (SSCP), and the like), that is linked to a particular transpiration efficiency phenotype.

The present invention clearly encompasses all interacting genes and/or alleles that are genetically linked to an ERECTA locus and are expressed to determine a transpiration efficiency phenotype. Such linked interacting genes and/or alleles will map to an ERECTA locus and be associated with the transpiration efficiency of that plant. Preferably, such interacting genes and/or alleles comprise a protein-encoding portion of a gene positioned within the ERECTA locus of the genome that is associated with the transpiration efficiency of that plant.

Homologs and/or orthologs of the exemplified alleles are clearly encompassed by the invention. Those skilled in the art are aware that the terms “homolog” and “ortholog” refer to functional equivalent units. In the present context, a homolog or ortholog of a gene that maps to an ERECTA locus shall be taken to mean any gene from a plant species that is functionally equivalent to a gene that maps to an exemplified ERECTA locus, and comprises a protein-encoding region in its native plant genome that shares a degree of structural identity or similarity with a protein-encoding region of the exemplified ERECTA gene.

Preferably, a homologous or orthologous gene from a plant other than A. thaliana will be associated with the transpiration efficiency of said plant and be linked to a protein-encoding region in its native plant genome that comprises a nucleotide sequence having at least about 55% overall sequence identity to a protein-encoding region linked to the ERECTA locus. Even more preferably, the percentage identity will be at least about 59-61% or 70% or 80%, still more preferably at least about 90%, and even still more preferably at least about 95%.

In determining whether or not two nucleotide sequences fall within a particular percentage identity limitation recited herein, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art. For example, nucleotide sequences may be aligned and their identity calculated using the BESTFIT program or other appropriate program of the Computer Genetics Group, Inc., University Research Park, Madison, Wis., United States of America (Devereaux et al, Nucl. Acids Res. 12, 387-395, 1984). In determining percentage identity of nucleotide sequences using a program known in the art or described herein, it is preferable that default parameters are used.

Alternatively, or in addition, a homologous or orthologous ERECTA or erecta allele will be associated with the transpiration efficiency of a plant and be linked to a protein-encoding region in its native plant genome that comprises a nucleotide sequence that encodes a polypeptide having at least about 55% overall sequence identity to a polypeptide encoded by a protein-encoding region linked to the ERECTA locus. Preferably, the percentage identity at the amino acid level will be at least about 59-61% or 70% or 80%, more preferably at least about 90%, and still more preferably at least about 95%.

In determining whether or not two amino acid sequences fall within these percentage limits, those skilled in the art will be aware that it is necessary to conduct a side-by-side comparison or multiple alignment of sequences. In such comparisons or alignments, differences will arise in the positioning of non-identical residues, depending upon the algorithm used to perform the alignment. In the present context, reference to a percentage identity or similarity between two or more amino acid sequences shall be taken to refer to the number of identical and similar residues respectively, between said sequences as determined using any standard algorithm known to those skilled in the art. For example, amino acid sequence identities or similarities may be calculated using the GAP program and/or aligned using the PILEUP program of the Computer Genetics Group, Inc., University Research Park, Madison, Wis., United States of America (Devereaux et al, 1984, supra). The GAP program utilizes the algorithm of Needleman and Wunsch, J. Mol. Biol. 48, 443-453, 1970, to maximize the number of identical/similar residues and to minimize the number and length of sequence gaps in the alignment. Alternatively or in addition, wherein more than two amino acid sequences are being compared, the ClustalW program of Thompson et al., Nucl. Acids Res. 22, 4673-4680, 1994, is used. In determining percentage identity of amino acid sequences using a program known in the art or described herein, it is preferable that default parameters are used.

Alternatively, or in addition, a homologous or orthologous ERECTA or erecta allele will be associated with the transpiration efficiency of a plant and be linked to a protein-encoding region in its native plant genome that hybridizes to nucleic acid that comprises a sequence complementary to a protein-encoding region linked to an ERECTA locus, such as, for example, from A. thaliana, rice, sorghum, maize, wheat or rice. Preferably, such homologs or orthologs will be identified by hybridization under at least low stringency conditions, and more preferably under at least moderate stringency or high stringency hybridization conditions.

For the purposes of defining the level of stringency, a low stringency is defined herein as being a hybridization or a wash carried out in 6×SSC buffer, 0.1% (w/v) SDS at 28° C. or alternatively, as exemplified herein. Generally, the stringency is increased by reducing the concentration of salt in the hybridization or wash buffer, such as, for example, by reducing the concentration of SSC. Alternatively, or in addition, the stringency is increased, by increasing the concentration of detergent (eg. SDS). Alternatively, or in addition, the stringency is increased, by increasing the temperature of the hybridization or wash. For example, a moderate stringency can be performed using 0.2×SSC to 2×SSC buffer, 0.1% (w/v) SDS, at a temperature of about 42° C. to about 65° C. Similarly, a high stringency can be performed using 0.1×SSC to 0.2×SSC buffer, 0.1% (w/v) SDS, at a temperature of at least 55° C. Conditions for performing nucleic acid hybridization reactions, and subsequent membrane washing, are well understood by one normally skilled in the art. For the purposes of further clarification only, reference to the parameters affecting hybridization between nucleic acid molecules is found in Ausubel et al., In: Current Protocols in Molecular Biology, Greene/Wiley, New York USA, 1992, which is herein incorporated by reference.

A number of mapping methods for determining useful loci and estimating their effects have been described (eg. Edwards et al., Genetics 116, 113-125, 1987; Haley and Knott, Heredity 69, 315-324, 1992; Jiang and Zeng, Genetics 140, 1111-1127, 1995; Lander and Botstein, Genetics 121, 185-199, 1989; Jansen and Stam, Genetics 136, 1447-1455, 1994; Utz and Melchinger, In: Biometrics in Plant Breeding: Applications of Molecular Markers. Proc. Ninth Meeting of the EUCARPIA Section Biometrics in Plant Breeding, 6-8 Jul. 1994, Wageningen, The Netherlands, (J. W. van Ooijen and J. Jansen, eds), pp 195-204, 1994; Zeng, Genetics 136, 1457-1468, 1994). In the present context, these methods are applied to identify the major component(s) of the total genetic variance that contribute(s) to the variation in transpiration efficiency of a plant, such as, for example, determined by the measurement of carbon isotope discrimination (Δ). More particularly, the segregation of known markers is used to map and/or characterize an underlying locus associated with transpiration efficiency. The locus method involves searching for associations between the segregating molecular markers and transpiration efficiency in a segregating population of plants, to identify the linkage of the marker to the locus.

To discover a marker/locus linkage, a segregating population is required. Experimental populations, such as, for example, an F2 generation, a backcross (BC) population, recombinant inbred lines (RIL), or double haploid line (DHL), can be used as a mapping population. Bulk segregant analysis, for the rapid detection of markers at specific genomic regions using segregating populations, is described by Michelmoore et al., Proc. Natl Acad. Sci. (USA) 88, 9828-9832, 1991. In the case of F2 mapping populations, F2 plants are used to determine genotype, and F2 families to determine phenotype. Recombinant inbred lines are produced by single-seed descent. Recombinant inbred lines, such as, for example, the F9 RILs of A. thaliana (eg. Lister and Dean, Plant J., 4, 745-750, 1993) will be known to those skilled in the art. Near isogenic lines (NILs) are used for fine mapping, and to determine the effect of a particular locus on transpiration efficiency. An advantage of recombinant inbred lines and double haploid lines is that they are permanent populations, and as a consequence, provide for replication of the contribution of a particular locus to the transpiration efficiency phenotype.

As for statistical methods, Single Marker Analysis (Point Analysis) is used to detect a locus in the vicinity of a single genetic marker. The mean transpiration efficiencies of a population of plants segregating for a particular marker, are compared according to the marker class. The difference between two mean transpiration efficiencies provides an estimate of the phenotypic effect of substituting one allele for another allele at the locus. To determine whether or not the inferred phenotypic effect is significantly different from zero, a simple statistical test, such as t-test or F-test, is used. A significant value indicates that a locus is located in the vicinity of the marker. Single point analysis does not require a complete molecular linkage map. The further the locus is from the marker, the less likely it is to be detected statistically, as a consequence of recombination between the marker and the gene.

In the Anova, t-test or GLM approach, the association between marker genotype and transpiration efficiency phenotype comprises:
(i) classifying progeny of a segregating population of plants by marker genotype, such as for example, using RFLP, AFLP, SSCP, or microsatellite analyses, thereby establishing classes of plants;
(ii) comparing the mean transpiration efficiencies of classes of plants in the segregating population, using a t-test, GLM or ANOVA; and
(iii) determining the significance of the differences in the mean at (ii), wherein a significant difference indicates that the marker is linked to the locus for transpiration efficiency.

As will be known to those skilled in the art, the difference between the means of the classes provides an estimate of the effect of the locus in determining the transpiration efficiency of a class.

In the regression approach, the association between marker genotype and phenotype is determined by a process comprising:
(i) assigning numeric codes to marker genotypes; and
(ii) determining the regression value r for transpiration efficiency against the codes, wherein a significant value for r indicates that the marker is linked to the locus for transpiration efficiency, and wherein the regression slope gives an estimate of the effect of a particular locus on transpiration efficiency.

For QTL interval mapping, the Mapmaker algorithm developed by Lincoln et al., Constructing genetic linkage maps with MAPMAKER/EXP version 3.0: A tutorial and reference manual. Whitehead Institute for Biomedical Research, Cambridge, Mass., USA, 1993, can be used. The principle behind interval mapping is to test a model for the presence of a QTL at many positions between two mapped marker loci. This model is a fit of a presumptive QTL to transpiration efficiency, wherein the suitability of the fit is tested by determining the maximum likelihood that a QTL for transpiration efficiency lies between two segregating markers. For example, in the case of a QTL located between two segregating markers, the 2-loci marker genotypes of segregating progeny will each contain mixtures of QTL genotypes. Accordingly, it is possible to search for loci parameters that best approximate the distribution in transpiration efficiency for each marker class. Models are evaluated by computing the likelihood of the observed distributions with and without fitting a QTL effect. The map position of a QTL is determined as the maximum likelihood from the distribution of likelihood values (LOD scores: ratio of likelihood that the effect occurs by linkage: likelihood that the effect occurs by chance), calculated for each locus.

Interval mapping by regression (Haley and Knott., Heredity 69, 315-324, 1992) is a simplification of the maximum likelihood method supra wherein basic QTL analysis or regression on coded marker genotypes is performed, except that phenotypes are regressed on the probability of a QTL genotype as determined from the linkage between transpiration efficiency and the nearest flanking markers. In most cases, regression mapping gives estimates of QTL position and effect that are almost identical to those given by the maximum likelihood method. The approximation deviates only at places where there are large gaps, or many missing genotypes.

In the composite interval mapping (CIM) method (Jansen and Stam, Genetics 136, 1447-1455, 1994; Utz and Melchinger, 1994, supra; Zeng, Genetics 136, 1457-1468, 1994), the analysis is performed in the usual way, except that the variance from other QTLs is accounted for by including partial regression giving more power and precision than simple interval mapping, because the effects of other QT1s are not present as residual variance. CIM can remove the bias that can be caused by the QTLs that are linked to the position being tested.

Publicly available software are used to map a locus for transpiration efficiency. Such software include, for example, the following:
(i) MapMaker/QTL (ftp://genome.wi.mit.edu/pub/mapmaker3/), for analyzing F2 or backcross data using standard interval mapping;
(ii) MQTL, for composite interval mapping in multiple environments or for performing simple interval mapping using homozygous progeny (eg. double haploids, or recombinant inbred lines);
(iii) PLABQTL (Utz and Melchinger, PLABlocus Version 1.0. A computer program to map QTL, Institut für Pflanzenzüchtung, Saatgutforschung and Populationsgenetik, Universität Hohenheim, 70593 Stuttgart, Germany, 1995; http://www.uni-hohenheim.de/˜ipspwww/soft.html) for composite interval mapping and simple interval mapping of a locus in mapping populations derived from a bi-parental cross by selling, or in double haploids;
(iv) QTL Cartographer (http://statgen.mcsu.edu/qticart/cartographer.html) for single-marker regression, interval mapping, or composite interval mapping, using F2 or backcross populations;
(v) MapQTL (http://www.cpro.dlo.nl/cbw/); Qgene for performing either single-marker regression or interval regression to map loci; and
(vi) SAS for detecting a locus by identifying associations between marker genotype and transpiration efficiency by a single marker analysis approach such as ANOVA, t-test, GLM or REG.

In a particularly preferred embodiment, QTL cartographer or MQTL is used to identify a locus associated with the transpiration efficiency of plants.

Those skilled in the art will also be aware that it is possible to detect multiple interacting alleles or genes for a particular trait, such as, for example, using composite interval mapping approaches. To achieve this end, the composite interval mapping may be repeated to look for additional loci. Alternatively, or in addition, two or more distinct regions of the genome can be nominated as candidate loci, and a gamete relationship matrix constructed for each candidate locus, and a 2-locus regression performed for each pair of loci, determining a best fit for the interacting effects between the two loci or aleles at those loci, including any dominance or additive effects. The algorithm described by Carlborg et al., Genetics (2000) can be used for simultaneous mapping. In the present context, such an analysis is performed with reference to the segregation of transpiration efficiency phenotypes in the segregating population.

Use of the ERECTA Locus to Enhance Transpiration Efficiency of Plants

As will be known to those skilled in the art, a single locus, if present in the genome of a plant, can have a significant influence on the phenotype of the plant. For example, Grandillo et al., Theor. Appl. Genet. 99, 978-987, 1999, showed that for tomato a selection made from a total 28 loci determining fruit size and weight explained 20% of the total phenotypic variance in this trait.

Accordingly, a second aspect of the invention provides a method of selecting a plant having enhanced transpiration efficiency, comprising:
(a) identifying a locus associated with genetic variation in transpiration efficiency in a plant; and
(b) selecting a plant that comprises or expresses a gene that maps to the locus.

By “enhanced transpiration efficiency” is meant that the plant loses less water per unit of dry matter produced, or alternatively, produces an enhanced amount of dry matter per unit of water transpired, or alternatively, fixes an increased amount of carbon per unit water transpired, relative to a counterpart plant. By “counterpart plant” is meant a plant having a similar or near-identical genetic background, such as, for example, a near-isogenic plant, a sibling, or parent.

In accordance with this aspect of the invention, a locus is identified by conventional locus mapping means, and/or by homology searching for genes that map to the ERECTA locus on chromosome 2 of the A. thaliana genome, such as, for example, by searching for ERECTA alleles or erecta alleles from a variety of plants, such as, for example, rice, wheat, sorghum, and maize, as described herein above.

Preferably, to select a plant that comprises or expresses the appropriate gene, marker-assisted selection (MAS) is used. As will be known to those skilled in the art, once a particular locus has been identified, genetic or physical markers that are linked to the locus can be readily identified and used to confirm the presence of the locus in breeding populations. For a locus that is flanked by two tightly-linked markers that recombine only at a low frequency, the presence of the flanking markers is indicative of the presence of the locus.

For marker-assisted selection, it is preferred that the marker is a genetic marker (eg. a gene or allele), or a physical marker (eg. leaf hairiness or pod shape), or a molecular marker such as, for example, a restriction fragment length polymorphism (RFLP), a restriction (RAPD), amplified fragment length polymorphism (AFLP), or a short sequence repeat (SSR) such as a microsatellite, or SNP. It is also within the scope of the invention to utilize any hybridization probe or amplification primer comprising at least about 10 nucleotides in length derived from a chromosome region that is linked in the genome of a plant to an ERECTA locus, as a marker to select plants. Those skilled in the art will readily be able to determine such probes or primers based upon the disclosure herein, particularly for those plant genomes which may have sufficient chromosome sequence in the region of interest in the genome (eg. A. thaliana, rice, cotton, barley, wheat, sorghum, maize, tomato, etc).

For flanking markers that are not tightly linked, such that there is a large recombination distance there between, the presence of the appropriate gene is assessed by identifying those plants having both flanking markers and then selecting from those plants having an enhanced transpiration efficiency. Naturally, the greater the distance between two markers, the larger the population of plants required to identify a plant having both markers, the intervening locus and a gene within said locus. Those skilled in the art will readily be able to determine the population size required to identify a plant having a particular transpiration efficiency, based upon the recombination units (cM) between two markers.

Transpiration efficiency is determined by any means known to the skilled artisan. Preferably, transpiration efficiency is determined by measuring dry matter accumulation in the plant by gravimetric means, or by measuring water loss, or the ratio of CO₂ assimilation rate to stomatal conductance.

In a particularly preferred embodiment, the transpiration efficiency is determined directly, by measuring the ratio of carbon fixed (carbon assimilation rate) to water loss (transpiration rate).

In an alternative embodiment, transpiration efficiency is determined indirectly from the carbon isotope discrimination value (Δ). Farquhar et al., Aust. J. Plant Physiol. 9,121-137, 1982, showed that carbon isotope discrimination (Δ; a measure of the extent to which the ¹³C/¹²C ratio of organic matter is less than that of CO₂ in the source air), is an effective indirect measure of transpiration efficiency. Discrimination, (Δ), is approximately the isotope ratio of carbon in source CO₂ minus that of plant organic carbon. In a particular experiment, the source CO₂ is common to all genotypes. The determination of transpiration efficiency in this manner is based upon the constancy of the atmospheric ¹³C:¹²C ratio (about 1.1:98.9) and the finding that ribulose bisphosphate carboxylase (Rubisco) enzymes discriminate against the use of ¹³C. Thus, in C₃ plants ¹³CO₂ is less efficiently assimilated than ¹²CO₂, and the ¹³CO₂ left behind tends to diffuse back through stomata in and out of the leaf. However, when the stomata become nearly closed, the relative back-diffusion of ¹³CO₂ is more difficult to achieve and the relative intracellular concentrations of ¹³CO₂ increases, thereby increasing the proportion of this isotope that is incorporated into 3-phosphoglycerate, and subsequently into dry matter. As a consequence, carbon isotope discrimination (Δ) is greatest when the overall CO₂ assimilation rate during photosynthesis (A) is small, and stomatal conductance (g_{w}) to water vapor is large. This relationship is represented by the following algorithm:

Δ(‰)=27−36A/(g_{w}×Cₐ)

wherein Cₐ is the ambient CO₂ concentration (ie. [¹²CO₂+¹³CO₂]). Discrimination, Δ, is approximately the isotope composition of source CO₂ minus that of plant organic carbon.

For a C₃ plant that exhibits a value in the range of about 4.5‰ to about 6.7‰ for the term 36A/(g_{w}×Cₐ), a 1‰ change in carbon isotope discrimination (Δ) corresponds to a change in transpiration efficiency in the range of about 22% to about 15%, respectively. The negative relationship between carbon isotope discrimination (Δ) and transpiration efficiency has been established for many C₃ plant species, including wheat (Farquhar and Richards, Aust. J. Plant Physiol. 11, 539-552, 1984; Farquhar et al., Ann. Rev. Plant Physiol. 40,388-397, 1989), Stylosanthes (Thumma et al., Proc. 9^{th} Aust. Agronomy Conf, Wagga Wagga New South Wales, Australia, 1998), cotton, barley, and rice. Accordingly, a lower carbon isotope discrimination (Δ) value for a test plant relative to a counterpart plant is indicative of enhanced transpiration efficiency.

In C₄ species, like maize, coefficients in the equation above are different (Farquhar 1983, Australian Journal of Plant Physiology, 10:205-226; Henderson et al.,1992, Aust. J. Plant Physiol. 19: 263-285):

Δ(‰)=1+5A/(g_{w}×Cₐ).

A 1‰ difference in Δ corresponds to about 38% difference in transpiration efficiency. The relationship between Δ and transpiration efficiency is positive. ¹³C preferentially accumulates in bicarbonate, the substrate for PEP carboxylation, and so discrimination against ¹³C is least when A is small and g_{w} is large. However, as CO₂ leakineess from the budle sheath increases, C₄ plants behave more like C₃ plants.

Alternatively, or in addition, transpiration efficiency is determined by another indicator, such as, for example, leaf temperature, ash content, mineral content, or specific leaf weight (dry matter per unit leaf area). For example, specific leaf weight is positively correlated with transpiration efficiency in peanuts and other species (Virgona et al., Aust. J. Plant Physiol., 17, 207-214, 1990; Wright et al., Crop Sci 34, 92-97, 1994). Accordingly, a higher specific leaf weight or higher carbon gain rate for a test plant relative to a counterpart plant is indicative of enhanced transpiration efficiency of the test plant.

The presence of the locus can be established by hybridizing a probe or primer that is linked to an ERECTA locus, such as, for example, a probe or primer that hybridizes to the identified chromosome 2 region of A. thaliana or the identified chromosome 6 region of rice.

Preferably, the presence of the locus is established by hybridizing a probe or primer derived from any one or more of SEQ ID Nos: 1, 3, 5, 7, 9, 11 to 19 or 21 to 44 or from a homologous gene in another plant, or a complementary sequence to such a sequence, to genomic DNA from the plant, and detecting the hybridization using a detection means.

In one embodiment, detection of the hybridization is performed preferably by labelling a probe with a reporter molecule capable of producing an identifiable signal, prior to hybridization, and then detecting the signal after hybridization. Preferred reporter molecules include radioactively-labelled nucleotide triphosphates and biotinylated molecules. Preferably, variants of the genes exemplified herein, including genomic equivalents, are isolated by hybridisation under moderate stringency or more preferably, under high stringency conditions, to the probe.

Alternatively, or in addition, hybridization may be detected using any format of the polymerase chain reaction (PCR), including AFLP. For PCR, two non-complementary nucleic acid primer molecules comprising at least about 20 nucleotides in length, and more preferably at least 30 nucleotides in length are hybridized to different strands of a nucleic acid template molecule, and specific nucleic acid molecule copies of the template are amplified enzymatically. Several formats of PCR are described in McPherson et al., In: PCR A Practical Approach., IRL Press, Oxford University Press, Oxford, United Kingdom, 1991, which is incorporated herein by reference.

For enhancing the transpiration efficiency of a plant wherein the locus is polymorphic, such as, for example, an allele, the method supra is modified to include the detection of the specific allele(s) linked to the desired enhancement. According to this embodiment, there is provided a method of selecting a plant having enhanced transpiration efficiency, comprising:
(d) identifying a locus associated with genetic variation in transpiration efficiency in a plant;
(e) identifying a polymorphic marker within said locus that is linked to enhanced transpiration efficiency; and
(f) selecting a plant that comprises or expresses the marker.

Standard means known to the skilled artisan are used to identify a marker within the locus that is linked to enhanced transpiration efficiency. A population of plants that is segregating for the polymorphic marker is generally used, wherein the transpiration efficiency phenotype of plants is then correlated or associated with the presence of a particular allelic form of the marker. As exemplified herein, near-isogenic or recombinant inbred lines of plants are screened to segregate alleles at the ERECTA locus and to correlate enhanced transpiration efficiency with the presence of the ERECTA allele as opposed to an erecta allele. Alternatively, mutations are introduced into an ERECTA allele such as, for example, by transposon mutagenesis, chemical mutagenesis or irradiation of plant material, and mutant lines of plants are established and screened to segregate alleles at the ERECTA locus that are correlated with the genetic variation in transpiration efficiency.

Suitable markers include any one or more of the markers described herein to be suitable for MAS.

Preferably, the selection of plants in accordance with these embodiments includes the additional step of introducing the locus or polymorphic marker to a plant, such as, for example, by standard breeding approaches or by recombinant means. This may be carried out at the same time, or before, selecting the locus or polymorphic marker.

Recombinant means generally include introducing a gene construct comprising the locus or marker into a plant cell, selecting transformed tissue and regenerating a whole plant from the transformed tissue explant. Means for introducing recombinant DNA into plant tissue or cells include, but are not limited to, transformation using CaCl₂ and variations thereof, in particular the method described by Hanahan (1983), direct DNA uptake into protoplasts (Krens et al, Nature 296, 72-74, 1982; Paszkowski et al., EMBO J. 3, 2717-2722, 1984), PEG-mediated uptake to protoplasts (Armstrong et al., Plant Cell Rep. 9, 335-339, 1990) microparticle bombardment, electroporation (Fromm et al., Proc. Natl. Acad. Sci. (USA), 82, 5824-5828, 1985), microinjection of DNA (Crossway et al., Mol. Gen. Genet. 202, 179-185, 1986), microparticle bombardment of tissue explants or cells (Christou et al, Plant Physiol. 87, 671-674, 1988; Sanford, Part. Sci. Technol. 5, 27-37, 1988), vacuum-infiltration of tissue with nucleic acid, or in the case of plants, T-DNA-mediated transfer from Agrobacterium to the plant tissue as described essentially by An et al., EMBO J. 4, 277-284, 1985; Herrera-Estrella et al., Herrera-Estella et al., Nature 303, 209-213, 1983; Herrera-Estella et al., EMBO J. 2, 987-995, 1983; or Herrera-Estella et al., In: Plant Genetic Engineering, Cambridge University Press, N.Y., pp 63-93, 1985.

For microparticle bombardment of cells, a microparticle is propelled into a cell to produce a transformed cell. Any suitable ballistic cell transformation methodology and apparatus can be used in performing the present invention. Exemplary apparatus and procedures are disclosed by Stomp et al. (U.S. Pat. No. 5,122,466) and Sanford and Wolf (U.S. Pat. No. 4,945,050). When using ballistic transformation procedures, the gene construct may incorporate a plasmid capable of replicating in the cell to be transformed.

Examples of microparticles suitable for use in such systems include 1 to 5 micron gold spheres. The DNA construct may be deposited on the microparticle by any suitable technique, such as by precipitation.

A whole plant may be regenerated from the transformed or transfected cell, in accordance with procedures well known in the art. Plant tissue capable of subsequent clonal propagation, whether by organogenesis or embryogenesis, may be transformed with a gene construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (eg., apical meristem, axillary buds, and root meristems), and induced meristem tissue (eg., cotyledon meristem and hypocotyl meristem).

The term “organogenesis”, as used herein, means a process by which shoots and roots are developed sequentially from meristematic centres.

The term “embryogenesis”, as used herein, means a process by which shoots and roots develop together in a concerted fashion (not sequentially), whether from somatic cells or gametes.

The generated transformed plants may be propagated by a variety of means, such as by clonal propagation or classical breeding techniques. For example, a first generation (or T1) transformed plant may be selfed to give homozygous second generation (or T2) transformant, and the T2 plants further propagated through classical breeding techniques.

The generated transformed organisms contemplated herein may take a variety of forms. For example, they may be chimeras of transformed cells and non-transformed cells; clonal transformants (eg., all cells transformed to contain the expression cassette); grafts of transformed and untransformed tissues (eg., in plants, a transformed root stock grafted to an untransformed scion).

Alternatively, the transformed plants are produced by an in planta transformation method using Agrobacterium tumefaciens, such as, for example, the method described by Bechtold et al., CR Acad. Sci. (Paris, Sciences de la vie/ Life Sciences) 316, 1194-1199, 1993 or Clough et al., Plant J 16: 735-74, 1998, wherein A. tumefaciens is applied to the outside of the developing flower bud and the binary vector DNA is then introduced to the developing microspore and/or macrospore and/or the developing seed, so as to produce a transformed seed. Those skilled in the art will be aware that the selection of tissue for use in such a procedure may vary, however it is preferable generally to use plant material at the zygote formation stage for in planta transformation procedures.

Identification of Genes for Determining the Transpiration Efficiency of a Plant

As exemplified herein, the inventors also identified specific genes or alleles that are linked to the ERECTA locus of A. thaliana, and rice and determine the transpiration efficiencies of those plants. More particularly, the transpiration efficiencies of near-isogenic lines, each carrying a mutation within an ERECTA locus, and a correlation between transpiration efficiency phenotype and ERECTA expression or gene copy number are determined, thereby providing the genetic contribution of genes or alleles at the ERECTA locus to transpiration efficiency. This analysis permits an assessment of the genetic contribution of particular alleles to transpiration efficiency, thereby determining allelic variants that are linked to a particular transpiration efficiency. Thus, the elucidation of the ERECTA locus for transpiration efficiency in plants facilitates the fine mapping and determination of allelic variants that modulate transpiration efficiency. The methods described herein can be applied to an assessment of the contribution of specific alleles to the transpiration efficiency phenotype for any plant species that is amenable to mutagenesis such as, for example, by transposon mutagenesis, irradiation, or chemical means known to the skilled artisan for mutating plants.

Accordingly, a third aspect of the invention provides a method of identifying a gene that determines the transpiration efficiency of a plant comprising:
(a) identifying a locus associated with genetic variation in transpiration efficiency in a plant;
(b) identifying a gene or allele that is linked to said locus, wherein said gene or allele is a candidate gene or allele for determining the transpiration efficiency of a plant; and
(c) determining the transpiration efficiencies of a panel of plants, wherein not all members of said panel comprise or express said gene or allele, and wherein variation in transpiration efficiency between the members of said panel indicates that said gene is involved in determining transpiration efficiency.

In another embodiment, the method comprises:
(a) identifying a locus associated with genetic variation in transpiration efficiency in a plant;
(b) identifying multiple alleles of a gene that is linked to said locus, wherein said gene is a candidate gene involved for determining the transpiration efficiency of a plant; and
(c) determining the transpiration efficiencies of a panel of plants, wherein each member of said panel comprises, and preferably expresses, at least one of said multiple alleles, wherein variation in transpiration efficiency between the members of said panel indicates that said gene is involved in determining transpiration efficiency.

In the present context, the term “near isogenic plants” shall be taken to mean a population of plants having identity over a substantial proportion of their genomes, notwithstanding the presence of sufficiently few differences to permit the contribution of a distinct allele or gene to the transpiration efficiency of a plant to be determined by a comparison of the transpiration efficiency phenotypes of the population. As will be known to the skilled artisan, recombinant inbred lines, lines produced by introgression of a gene or transposon followed by several generations of backcrossing, or siblings, are suitable near-isogenic lines for the present purpose.

Preferably, the identified gene or allele identified by the method described in the preceding paragraph is selected from the group consisting of ERECTA allele, erecta allele, and homologs of ERECTA, wherein said homologs are from plants species other than A. thaliana.

In a particularly preferred embodiment, the identified gene or allele will comprise a nucleotide sequence selected from the group consisting of:
(d) a sequence having at least about 55% identity to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44;;
(e) a sequence encoding an amino acid sequence having at least about 55% identity to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45; and
(f) a sequence complementary to (a) or (b).

Preferably, the percentage identity is at least about 59-61% or 70% or 80%, more preferably at least about 90%, and even more preferably at least about 95% or 99%. In a particularly preferred embodiment, the identified gene or allele comprises a nucleotide sequence selected from the group consisting of:
(a) a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44;
(b) a sequence encoding an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45; and
(c) a sequence complementary to (a) or (b).

Enhancement of Transpiration Efficiency Using Isolated Genes

The identified gene or alleles, including any homologs from a plant other than A. thaliana, such as, for example, the wild-type ERECTA allele, or a homolog thereof, is useful for the production of novel plants. Such plants are produced, for example, using recombinant techniques, or traditional plant breeding approaches such as by introgression.

Accordingly, a fourth aspect of the present invention provides a method of enhancing the transpiration efficiency of a plant comprising ectopically expressing in a plant an isolated ERECTA gene or an allelic variant thereof or the protein-encoding region thereof.

Preferably, the ERECTA gene or allelic variant comprises a nucleotide sequence that is homologous to a protein-encoding region of a gene that is linked to the A. thaliana ERECTA locus on chromosome 2.

In a particularly preferred embodiment, the isolated gene comprises a nucleotide sequence selected from the group consisting of:
(a) a sequence having at least about 55% identity to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44;
(b) a sequence encoding an amino acid sequence having at least about 55% identity to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45; and
(c) a sequence complementary to (a) or (b).

Preferably, the percentage identity is at least about 59-61% or 70% or 80%, more preferably at least about 90%, and even more preferably at least about 95% or 99%.

In a particularly preferred embodiment, the isolated gene or allele comprises a nucleotide sequence selected from the group consisting of:
(a) a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44;
(b) a sequence encoding an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45; and
(c) a sequence complementary to (a) or (b).

To ectopically express the isolated gene in a plant, the protein-encoding portion of the gene is generally placed in operable connection with a promoter sequence that is operable in the plant, which may be the endogenous promoter or alternatively, a heterologous promoter, and a transcription termination sequence, which also may be the endogenous or an heterologous sequence relative to the gene of interest. The promoter and protein-encoding portion and transcription termination sequence are generally provided in the form of a gene construct, to facilitate introduction and maintenance of the gene in a plant where it is to be ectopically expressed. Numerous vectors suitable for introducing genes into plants have been described and are readily available. These may be adapted for expressing an isolated gene in a plant to enhance transpiration efficiency therein.

Reference herein to a “promoter” is to be taken in its broadest context and includes the transcriptional regulatory sequences of a classical eukaryotic genomic gene, including the TATA box which is required for accurate transcription initiation, with or without a CCAAT box sequence and additional regulatory elements (ie. upstream activating sequences, enhancers and silencers) which alter gene expression in response to developmental and/or external stimuli, or in a tissue-specific manner. In the present context, the term “promoter” is also used to describe a synthetic or fusion molecule, or derivative which confers, activates or enhances expression of said sense molecule in a cell. Preferred promoters may contain additional copies of one or more specific regulatory elements, to further enhance expression and/or to alter the spatial expression and/or temporal expression of a nucleic acid molecule to which it is operably connected. For example, copper-responsive regulatory elements may be placed adjacent to a heterologous promoter sequence driving expression of a nucleic acid molecule to confer copper inducible expression thereon.

Placing a nucleic acid molecule under the regulatory control of a promoter sequence means positioning said molecule such that expression is controlled by the promoter sequence. A promoter is usually, but not necessarily, positioned upstream or 5′ of the protein-encoding portion of the gene that it regulates. Furthermore, the regulatory elements comprising a promoter are usually positioned within 2 kb of the start site of transcription of the structural protein-encoding nucleotide sequences, or a chimeric gene comprising same. In the construction of heterologous promoter/structural gene combinations it is generally preferred to position the promoter at a distance from the gene transcription start site that is approximately the same as the distance between that promoter and the gene it controls in its natural setting, ie., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, ie., the genes from which it is derived. Again, as is known in the art, some variation in this distance can also occur.

Promoters suitable for use in gene constructs of the present invention include those promoters derived from the genes of viruses, yeasts, moulds, bacteria, insects, birds, mammals and plants which are capable of functioning in plant cells, including monocotyledonous or dicotyledonous plants, or tissues or organs derived from such cells. The promoter may regulate gene expression constitutively, or differentially with respect to the tissue in which expression occurs or, with respect to the developmental stage at which expression occurs, or in response to external stimuli such as physiological stresses, pathogens, or metal ions, amongst others.

Examples of promoters useful in performing this embodiment include the CaMV 35S promoter, rice actin promoter, rice actin promoter linked to rice actin intron (PAR-IAR) (McElroy et al, Mol and Gen Genetics, 231(1), 150-160, 1991), NOS promoter, octopine synthase (OCS) promoter, Arabidopsis thaliana SSU gene promoter, napin seed-specific promoter, PcSVMV, promoters capable of inducing expression under hydric stress, as described by, for example, Kasuga et al, Nature Biotechnology, 17, 287-291, 1999), SCSV promoter, SCBV promoter, 35s promoter (Kay et al, Science 236, 4805, 1987) and the like. In addition to the specific promoters identified herein, cellular promoters for so-called housekeeping genes, including the actin promoters, or promoters of histone-encoding genes, are useful.

The term “terminator” refers to a DNA sequence at the end of a transcriptional unit which signals termination of transcription. Terminators are 3′-non-translated DNA sequences containing a polyadenylation signal, that facilitate the addition of a polyadenylate sequence to the 3′-end of a primary transcript. Terminators active in cells derived from viruses, yeasts, moulds, bacteria, insects, birds, mammals and plants are known and described in the literature. They are isolatable from bacteria, fungi, viruses, animals and/or plants.

Examples of terminators particularly suitable for use in the gene constructs of the present invention include the nopaline synthase (NOS) gene terminator of Agrobacterium tumefaciens, the terminator of the Cauliflower mosaic virus (CaMV) 35S gene, the zein gene terminator from Zea mays, the Rubisco small subunit (SSU) gene terminator sequences and subclover stunt virus (SCSV) gene sequence terminators, amongst others.

Those skilled in the art will be aware of additional promoter sequences and terminator sequences that may be suitable for use in performing the invention. Such sequences may readily be used without any undue experimentation.

Preferably, the gene construct further comprises an origin of replication sequence for its replication in a specific cell type, for example a bacterial cell, when said gene construct is required to be maintained as an episomal genetic element (eg. plasmid or cosmid molecule) in said cell. Preferred origins of replication include, but are not limited to, the f1-ori and colE1 origins of replication.

Preferably, the gene construct further comprises a selectable marker gene or genes that are functional in a cell into which said gene construct is introduced.

As used herein, the term “selectable marker gene” includes any gene which confers a phenotype on a cell in which it is expressed to facilitate the identification and/or selection of cells which are transfected or transformed with a gene construct of the invention or a derivative thereof.

Suitable selectable marker genes contemplated herein include the ampicillin resistance (Amp^{r}), tetracyclin-resistance gene (Tc^{r}), bacterial kanamycin resistance gene (Kan^{r}), phosphinothricin resistance gene, neomycin phosphotransferase gene (nptII), hygromycin resistance gene, gentamycin resistance gene (gent), β-glucuronidase (GUS) gene, chloramphenicol acetyltransferase (CAT) gene, and luciferase gene, Green Fluorescent Protein gene (EGFP and variants), amongst others.

In a related embodiment, the invention extends to the use of an isolated gene comprising a nucleotide sequence that is homologous to a protein-encoding region of a gene of A. thaliana that is positioned between about 46 cM to about 50.74 cM on chromosome 2 in the preparation of a gene construct for enhancing the transpiration efficiency of a plant.

In an alternative embodiment of the invention, the transpiration efficiency of a plant is enhanced by classical breeding approaches, comprising introgressing the isolated gene into a plant. For introgression of a gene, the gene is transferred from its native genetic background into another genetic background using standard breeding, for example, a gene that enhances transpiration efficiency in a progenitor such as a diploid cotton or diploid wheat may be transferred into a commercial tetraploid cotton or hexaploid wheat, respectively, by standard crossing, followed by several generations of back-crossing to remove the genetic background of the progenitor. Naturally, continued selection of the gene of interest is required, such as, for example, facilitated by the use of markers.

A further aspect of the present invention provides a plant having enhanced transpiration efficiency, wherein said plant is produced by a method described herein.

Clearly the ERECTA genes, allelic variants and protein coding regions described herein are useful in determining other proteins that are involved in the transpiration process in plants. For example, an ERECTA gene, allelic variant thereof or protein coding region thereof may be used in a forward ‘n’-hybrid assay to determine if said peptide is able to bind to a protein or peptide of interest. Forward ‘n’ hybrid methods are well known in the art, and are described for example, by Vidal and Legrain Nucl. Acid Res. 27(4), 919-929 (1999) and references therein, and include yeast two-hybrid, bacterial two-hybrid, mammalian two-hybrid, PolIII (two) hybrid, the Tribrid system, the ubiquitin based split protein sensor system and the SOS recruitment system. Such methods are incorporated herein by reference

In adapting a standard forward two-hybrid assay to the present invention, an ERECTA protein is expressed as a fusion protein with a DNA binding domain from, for example, the yeast GAL4 protein. Methods of constructing expression constructs for the expression of such fusion proteins are well known in the art, and are described, for example, in Sambrook et al (In: Molecular Cloning: A laboratory Manual, Cold Spring Harbour, N.Y., Second Edition, 1989). A second fusion protein is also expressed in the yeast all, said fusion protein comprising, for example, a protein thought to interact with an ERECTA protein, for example the GAL4 activation domain. These two constructs are then expressed in a yeast cell in which, a reporter molecule (e.g., tet^{r}, Amp^{r}, Rif^{r}, bsdf^{r}, zeof^{r}, Kan^{r}, gfp, cobA, LacZ, TRP1, LYS2, HIS3, HISS, LEU2, URA3, ADE2, MET13, MET15) under the control of a minimal promoter placed in operable connection with a GAL 4 binding site. If the proteins do not interact, a reporter molecule is not expressed. However, if said proteins do interact, said reporter molecule is expressed. Accordingly a protein, polypeptide, peptide that is able to specifically bind a target protein is identified.

A forward ‘n’-hybrid method may be modified to facilitate high throughput screening of a library of peptides, polypeptides and/or proteins in order to determine those that interact with an ERECTA protein. Methods of screening libraries of proteins are well known in the art and are described, for example, in Scopes (In: Protein Purification: Principles and Practice, Third Edition, Springer Verlag, 1994). Proteins identified by this method are potentially involved in the transpiration process in plants.

The present invention is further described with reference to the following non-limiting examples.

### Example 1

### ¹²C/¹³C Discrimination as a Marker for Screening Genetic Variation in Transpiration Efficiency

Experimental conditions and sampling procedures were established to allow the control of many factors, other than genetic, that influence transpiration efficiency at the level of individual leaves and plants. These factors fall into several categories: (a) characteristics of the seedling's micro-environment: temperature, light, humidity, boundary layer around the leaves, root growth conditions; (b) developmental and morphological effects that modify gas exchange and C metabolism and therefore carbon isotopic signature (eg age, stage, posture); and (c) seed effects.

We developed high resolution mass-spectrometer techniques for measuring C isotope ratios in whole tissues or carbon compounds such as soluble sugars—ie a measure of integrated transpiration efficiency over the plant's life or over a day, respectively, and also for measuring instantaneous transpiration efficiency during gas exchange.

This means:
0.1 per mil analytical precision in the measurement of the isotopic composition of leaf carbon. Discrimination, (Δ), is approximately the isotope ratio of carbon in source CO₂ minus that of plant organic carbon. In a particular experiment, the source CO₂ is common to all genotypes.
0.1 per mil biological precision, that is variation between replicated seedlings, grown in soil, either in growth chambers or in glasshouses with CO₂, humidity and temperature control (corresponding to approximately 1.5% variation in transpiration efficiency).
The ability to grow and screen large batches of seedlings in glasshouses or growth chambers (up to 1500), under standardised leaf and root growth conditions, to a rosette size of several cm within 2-3 weeks allowing individual measurements, on the same plant, of isotope ratios and also of the underlying properties (eg in situ measurement of leaf temperature by infra-red thermometry as a measure of stomatal conductance; chlorophyll fluorescence; leaf expansion).

### Example 2

### Natural Genetic Variation in Transpiration Efficiency in Arabidopsis thaliana

A. thaliana ecotypes were screened for leaf A under glasshouse conditions. There was a large spread of values (corresponding to approximately 30% genetic variation in transpiration efficiency). However, large environmental effects were noted. A few contrasted ecotypes were selected at the two extremes of the range of Δ values and compared under various conditions of irradiance (150 to 500 μE m⁻²s⁻¹), light spectrum (Red/Far-Red ratios) and air humidity (60 to 90%) while roots were always well watered. The magnitude of genetic differences in transpiration efficiency was very much influenced by environmental conditions. This was in part due to variations among ecotypes in the dependence of photosynthesis on light and vapour pressure deficit. Genetic differences were maximal under a combination of high light and low humidity, in growth chambers.

The ecotypes Columbia (Col) and Landsberg erecta (Ld-er) have extreme carbon isotope discrimination values, with Col always having smaller Δ values than Ld-er ie less negative δ¹³C isotopic compositions, and thus a greater transpiration efficiency.

### Example 3

### Identification of a Locus Associated with Transpiration Efficiency in A. thaliana

Quantitative Trait Loci (QTL) analysis of the Lister and Dean's (1993) Recombinant Inbred Lines (later referred to as RILs) was performed to identify and map a locus associated with carbon isotope discrimination (Δ). The RILs were from a cross between Col-4 and Ler-0. Our analysis showed the importance of genes around the ER locus on chr2, and a role for genes other than ERECTA in conferring transpiration efficiency on A. thaliana.

More particularly, 300 RI mapping lines between Col and Ler ecotypes, available at the Arabidopsis Stock Centre, were generated from a cross between the Arabidopsis ecotypes Columbia (Col4) and Landsberg erecta (Ler-0 carrying er1) (Lister and Dean, 1993), using Columbia as the male parent. A subset of 100 of these lines, chosen as the most densely and reliably mapped were used in the present analysis.

The seeds were multiplied in a glasshouse in an attempt to minimize confounding seed effects in our comparisons. Large numbers of seeds were obtained for most lines except for a few, including Col4 parent, which had to be re-ordered following low seed viability of the original sample sent by the Stock Centre. The seeds harvested in these propagation runs were used throughout all our experiments to date.

Loci were analysed using two programs, QTL cartographer and MQTL. These programs compute statistics of a trait at each marker position, using a range of methods [linear regression (LR), stepwise regression (SR), and likelihood approaches (Single interval mapping (SIM) which treats values at individual markers as independent values, and composite interval mapping (CIM) which allows for interactions between markers and associated locus)]. By nature each of these methods has some biases and embedded assumptions, hence the importance of analysing data with more than one program. Only results that were consistent between the two programs, and robust to additions or deletions to the set of background markers used for composite interval mapping are reported below.

Initial QTL analysis was done in parallel to seed multiplication on a subset of 40 lines for which enough seeds were sent. Once all seeds had been multiplied this was repeated on the full set of 100 lines. These two analyses indicated the existence of a locus for carbon isotope discrimination (Δ), that maps to the region including the ERECTA locus on chromosome 2, at approximately 46-51 cM (Table 1, run 1&2).

Given the complexity and integrative nature of A as a physiological trait, such a small number of loci associated with the trait was not expected. Subsequent experiments were therefore designed to test these results and assess their stability across the range of environmental conditions known for their effects on gene expression related to A (see above). QTL analysis was repeated on several completely independent data sets obtained under highly controlled conditions in glasshouses or growth chambers, where either air humidity, photoperiod or irradiance (amount, diurnal pattern, day to day variation) was varied. Depending on the experiment, all 100 recombinants inbred lines were included or only the subset of lines with cross-overs on chromosome 2. These experiments confirmed that genetic variation in A could be mostly ascribed to a portion of chromosome 2 (Table 1) between about 46-50.7 cM.

When RILs were sorted graphically according to carbon isotope discrimination and their genotype at the ER marker (50.64 cM) and its vicinity (Ld-er1 genotype or Col-ER genotype), lines which were Ld-er at the ERECTA marker ranked mostly at the high end of carbon isotope discrimination values. In contrast, lines having a Col-ERECTA marker genotype ranked mostly at the low end of carbon isotope discrimination values (data available on request). In the middle of the range of carbon isotope discrimination values, there was some overlap between the two sets of lines. Some lines were always at an extreme (in all 18 experiments performed), while the ranking of other lines was more unstable. These data indicate a locus for transpiration efficiency, as determined by the carbon isotope discrimination value, in the vicinity of the ERECTA locus on chromosome 2 (Table 1). This locus most likely involves the ER gene. Depending on the positions of cross-overs between Ld-er and Col, recombination between ERECTA and one or more of the other genes influences the transpiration efficiency phenotype of the progeny.

### Example 4

### Transformation Protocol for Maize

Gun Transformation

A suitable method for maize transformation is based on the use of a particle gun identical to that described by J. Finer (1992, Plant Cell Report, 11:323-328). The target cells are fast dividing undifferentiated cells having maintained a capacity to regenerate in whole plants. This type of cells composes the embryogenic callus (called type II) of maize. These calluses are obtained from immature embryos of genotype HiII according to the method and on medium described by Armstrong (Maize Handbook; 1994 M. Freeling, V. Walbot Eds; pp. 665-671).

These fragments of the calluses having a surface from 10 to 20 mm2 are arranged, 4 hour before bombardment, by putting 16 fragments by dish in the center of a Petri dish containing an culture medium identical to the medium of initiation of calluses, supplemented with 0.2 M of mannitol+0.2 M of sorbitol. Plasmids containing the ERECTA sequences to be introduced, are purified on Qiagen® column following the instructions of the manufacturer.

They are then precipitated on particles of tungsten (M10) following the protocol described by Klein et al, Nature, 327, 70-73, (1987). Particles so coated are sent towards the target cells by means of the gun according to the protocol described by Finer et al, Plant Cell Report, 11:323-328, 1992. The bombarded dishes of calluses are then sealed by means of Scellofrais® then cultivated in the dark at 27° C.

The first transplanting takes place 24 hours later, then every other week during 3 months on medium identical to the medium of initiation supplemented with a selective agent. After 3 months or sometimes earlier, one can obtain calluses the growth of which is not inhibited by the selective agent, usually and mainly consisting of cells resulting from the division of a cell having integrated into its genetic patrimony one or several copies of the gene of selection. The frequency of obtaining of such calluses is about 0.8 callus by bombarded dish.

These calluses are identified, individualized, amplified then cultivated so as to regenerate seedlings, by modifying the hormonal and osmotic equilibrium of the cells according to the method described by Vain and al. (1989, Plant Cell tissue and organ Culture 18:143-151). These plants are then acclimatized in greenhouse where they can be crossed for obtaining hybrids or self-fertilized.

In a preferential way, one can use a similar protocol, the principle of which is described in Methods of Molecular Biology: Plant gene transfer and expression protocols (1995, vol. 49, PP 113-123), and in which the immature embryos of genotype HiII are directly bombarded with golden particles coated with plasmides ERECTA to introduce, prepared according to the protocol described by Barcelo and Lazzeri (1995, Methods of Molecular Biology, 49:113-123).

Steps of transformation, selection of the events, maturation and regeneration are similar to those described in the previous protocol.

Agrobacterium Transformation

Another technique of useful transformation within the framework of the invention uses Agrobacterium tumefaciens, according to the protocol described by Ishida and al (1996, Nature Biotechnology 14: 754-750), in particular starting from immature embryos taken 10 days after fertilization.

All the used media are referenced in the quoted reference. The transformation begins with a phase of co-culture where the immature embryos of the maize plants are put in contact during at least 5 minutes with Agrobacterium tumefaciens LBA 4404 containing the superbinary vectors.

The superbinary plasmid is the result of an homologous recombination between an intermediate vector carrying the T-DNA, and containing the gene of interest and/or the marker gene of selection, and the vector pSB1 of Japan Tobacco (EP 672 752) containing: the virB and virG genes of the plasmide pTiBo542 present in the supervirulent strain A281 of Agrobacterium tumefaciens (ATCC 37349) and an homologous region found in the intermediate vector, allowing homologous recombination.

Embryos are then placed on LSAs medium for 3 days in the dark and at 25° C. A first selection is made on the transformed calluses: embryogenic calluses are transferred on LSD5 medium containing phosphinotricine (5 mg/l) and céfotaxime (250 mg/l) (elimination or limitation of contamination by Agrobacterium tumefaciens).

This step is performed during 2 weeks in the dark and at 25° C. The second step of selection is realized by transfer of the embryos which developed on LSD5 medium, on LSD10 medium (phosphinotricine, 10 mg/l) in the presence of céfotaxime, during 3 weeks at the same conditions as previously. The third stage of selection consists in excising the calluses of type I (fragments from 1 to 2 mm) and in transferring them for 3 weeks in the darkness and at 25° C. on LSD 10 medium in the presence of céfotaxime. The regeneration of seedlings is made by excising the calluses of type I which proliferated and by transferring them on LSZ medium in the presence of phosphinotricine (5 mg/l) and of céfotaxime for 2 weeks at 22° C. and under continuous light.

Seedlings having regenerated are transferred on RM medium+G2 containing Augmentin (100 mg/l) for 2 weeks at 22° C. and under continuous illumination for the development step. The obtained plants are then transferred to the phytotron with the aim of acclimatizing.

### Example 5

### Detecting Expression of ERECTA Protein

Extraction of ERECTA from Leaves and Seeds of Maize.

Leaves are harvested and immediately frozen in liquid nitrogen. Grinding is made in a mortar cleaned in ethanol 100% and cooled on ice. A foliar disc of 18 mm diameter is extracted in 200 μL of extraction buffer: Tris-HCl pH 8.0, glycerol 20%, MgCl2 10 mM, EDTA 1 mM, DTT 1 mM, PVP insoluble 2% (p/v), Fontainebleau sand et protease inhibitors: leupeptin 2 mg/L, chymostatin 2 mg/L, PMSF 1 mM and E64 1 mg/L. The ground material is then centrifuged in 4° C. during 15 minutes at 20000 g to eliminate fragments.

Grains are first reduced to powder in a bead-crusher (Retsch). Proteins are extracted by suspending 100 μL of powder in 400 μL of the previously described buffer on ice. This mixture is vortexed and centrifuged at 4° C. during 15 minutes et 20000 g to eliminate fragments.

ERECTA protein levels are then measured using techniques known to those skilled in the art, and described, for example, in Scopes (In: Protein purification: principles and practice, Third Edition, Springer Verlag, 1994).

### Example 6

### Determination of a Role for the ERECTA Gene in Regulating Transpiration Efficiency

We compared Col and Ler ecotypes with near-isogenic mutant lines for the erecta gene, to examine a possible role of the ERECTA gene in determining carbon isotope discrimination (Δ).

Plants expressing the wild type ERECTA gene (SEQ ID NO: 1), or an erecta mutant allele in the Columbia background (eg. Col-er1, Col-er2, Col-er101 to -er105; or Col-er106 to -er123) and in Landsberg background (Ld-er1) have been publicly described. Three of these mutants were available for comparison to the isogenic or near-isogenic lines (Table 2).

Col4 (ER) and Ld-er1, the parental lines for Lister and Dean's R1Ls were systematically included in the comparison. Where possible, other Col “ecotypes” were also included, (eg. Col0, Col1, Col3-7), to assess their similarity with respect to carbon isotope discrimination, especially compared to the RIL parental ecotype Col4.

The results of these comparisons are described in Table 3. Data indicate the differences in carbon isotope discrimination values between er and ER lines for 15 different experimental runs corresponding to growth under low to high light (100 to 800 μE m⁻² s⁻¹), low to high humidity (40 to 85%), short to long days (8, 10, 24 hrs), normal to high temperatures (22/20° C. to 28/20° C.).

As expected, the spread of carbon isotope discrimination values among lines varied with environmental conditions. Lines carrying er mutations have a greater carbon isotope discrimination value overall than those having the ER wild type gene (see Table 3, column 1), indicative of a lower water use-efficiency. There is usually little difference in C isotopic discrimination between the various Col lines, (see the similar averages obtained for columns 2, 3, and 4 in Table 3, wherein er105 is compared to 3 different Col ecotypes, Col0, Col4 and 3176 or Col1). When present, the er105 mutant always has the greatest carbon isotope discrimination value of all lines, including er1 and er2 (columns 2-4 compared to columns 5-6 in Table 3, or column 8 compared to column 9 in Table 3). The value measured in the er105 mutant is always significantly greater than in the ER isogenic line (column 4 in Table 3). The value measured in er1 (Landsberg parental line NW20) is usually also greater than that in the ER lines 3177 (near isogenic, column 6 of Table 3), and to a lesser extent Col4 (Columbia parental line, column 7 of Table 3). These observations give direct evidence that the ERECTA gene plays a significant role in determining genetic differences in carbon isotopic discrimination in Arabidopsis.

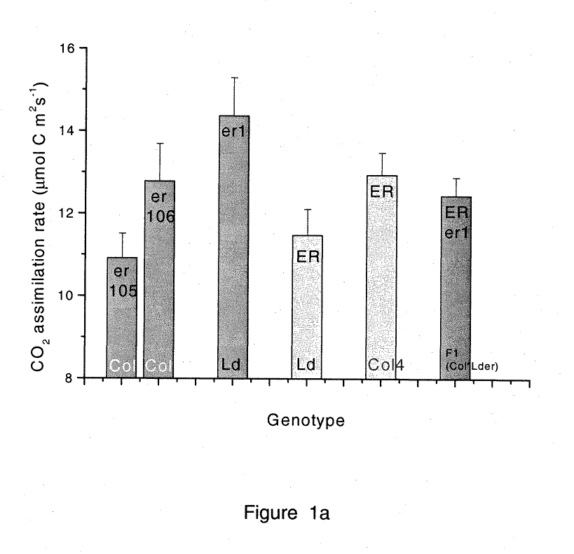

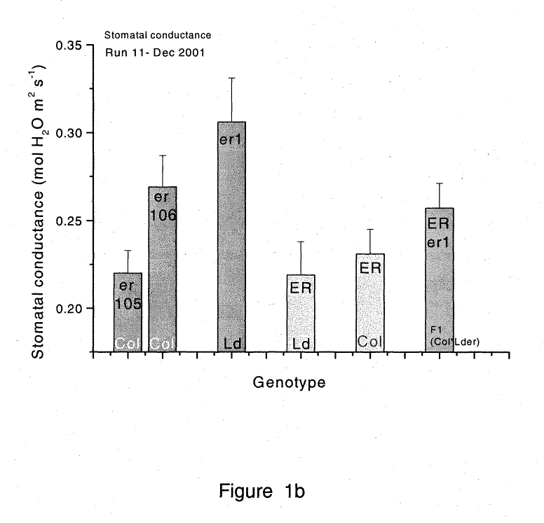

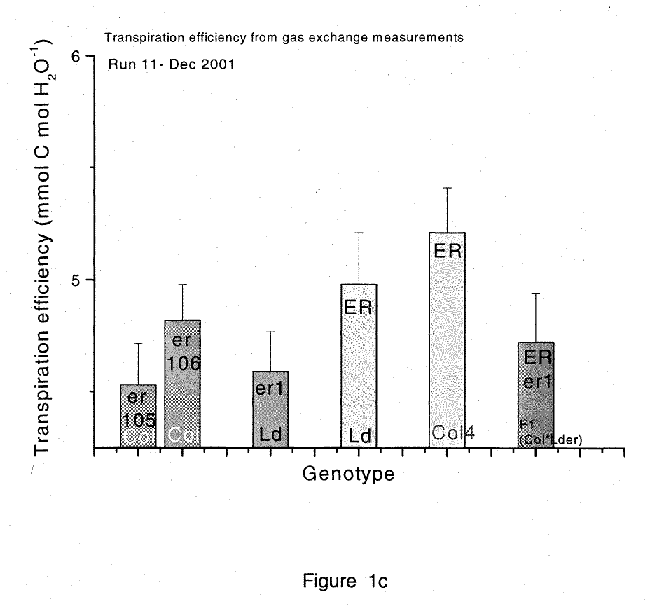

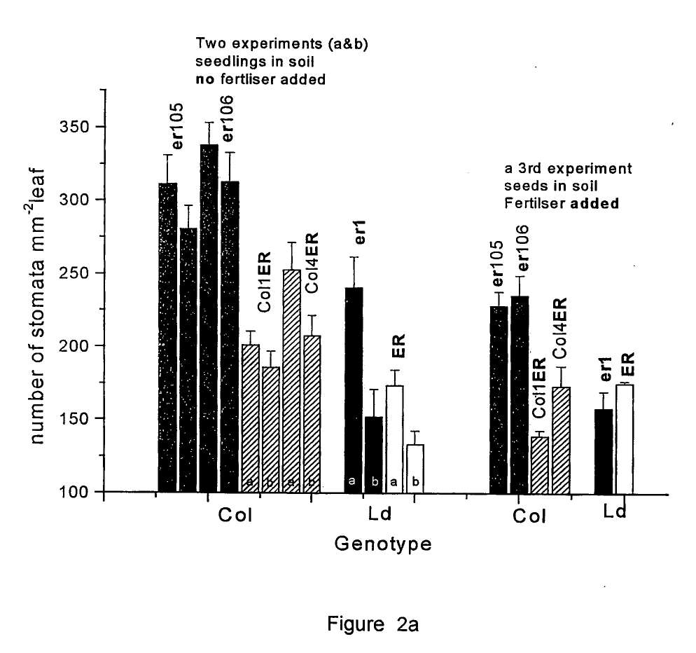

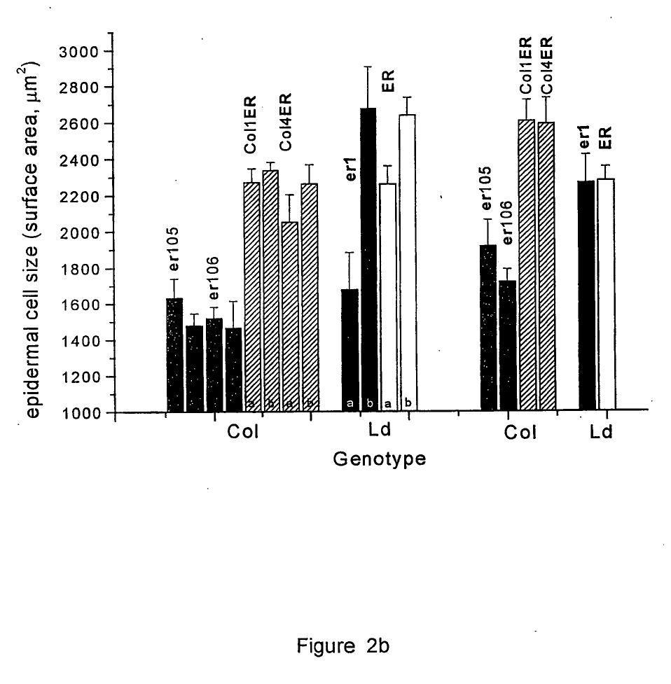

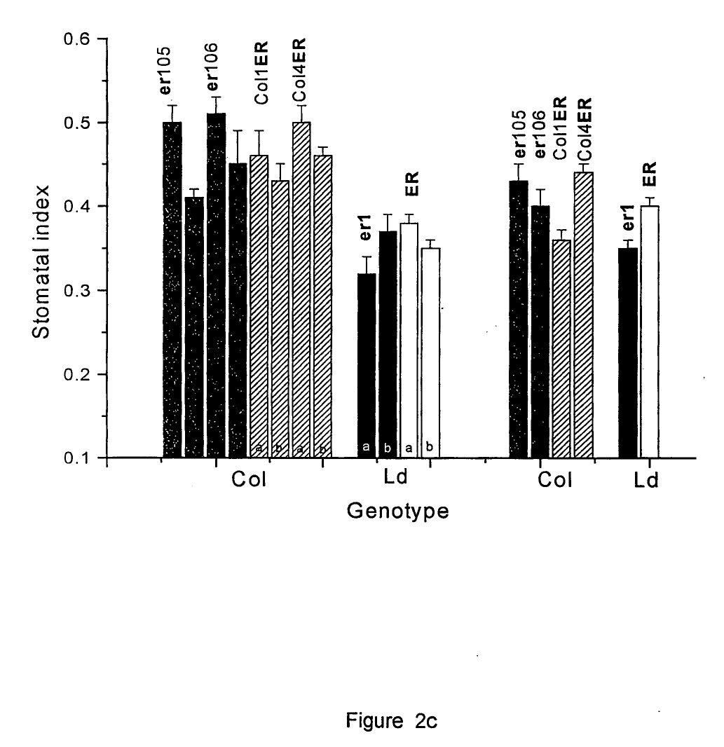

This conclusion is independently confirmed by leaf gas exchange measurements that allow the direct measure of transpiration efficiency (ratio of net CO₂ fixation to water loss; column 4 in Table 4; FIGS. 1a-1c, 2a-2c). Measurements on mature leaves reveal that ER lines are characterised by a greater ratio of CO₂ assimilation to water loss than lines carrying er mutations. This is most obvious when comparing the pair Col1/er105 with a 21% greater transpiration efficiency (ratio A/E) in Col1 than er105, or the pair Col1/er2 with a 16% greater transpiration efficiency in Col1. Consistent with the measurements of carbon isotope discrimination, the effect er/ER is relatively smaller in the Ld background (9% greater ratio A/E in Ld-ER (3177) than in the Ld-er1 (NSW20) line).

Also consistent with the carbon discrimination measurements, is the 20% difference in transpiration efficiency between the two RILs parental lines (4.06 and 3.38 mmolC/molH2O in Col4-ER and Ld-er1, respectively).

The fact that of all 3 erecta mutants examined, er105 has the most extreme carbon discrimination and transpiration efficiency phenotypes suggests that the er105 mutation affects a more crucial part of the ERECTA gene than er2 or er1. This is consistent with the published data on the er105 mutant. This mutation corresponds to the insertion of a large “foreign insert” in the ERECTA gene. The insertion inhibits transcription of the gene and causes the strongest erecta phenotype of all erecta mutants isolated in Col (with respect to inflorescence clustering and silique width and shape). Alternatively, or in addition, data indicate that erecta mutations have a stronger effect on carbon isotope discrimination values in a Columbia genetic background than in a Landsberg background (comparison of phenotypic effects of er105 and er1), implying that other genes, polymorphic between Landsberg and Columbia ecotypes, interact with ERECTA in determining transpiration efficiency. This could also account for the greater difference in transpiration efficiency between er/ER lines in Col background than in a Ld background (see above, Table 4). Alternatively, or in addition, data indicate that the erecta mutation is not the only mutation present in the er105 mutant. For example, the mutagenized Col seeds may have carried the gl1 mutation, induced by the fast neutron irradiation, that also contributes to the phenotype observed.

A comparison of transcript profiles in er/ER isogenic lines (in both Col and Ld background) allows determination of the involvement of additional genes to ERECTA and the effect of environment on their expression.

### Example 7

### QTL Detection Centred on the ERECTA Marker and ERECTA Gene Locus on Chromosome 2 of Arabidopsis thaliana

1. Methods

Numerous runs using the Lister and Dean (1993) Recombinant Inbred Lines between Col-4 and Ler-0 were grown in a temperature controlled glass house (20/20° C.) or within growth cabinets (21° C. and light levels ranging from 100 to 500 μE m⁻² s⁻¹ irradiance, and 50-70% relative humidity). Runs included a variety of all 100 RILs as well as subsets of these 100 along with parental Col-4 and Ler-0 (NW20) parental lines. Individual RILs were replicated within runs. Seeds were either cold-treated on moist filter paper for 2-4 days, cold-treated and planted directly onto soil; or plated onto agar, cold-treated for 2-4 days, grown on agar for about 11-15 days before being transferred to soil. Plants were well-watered, and grown for 4-5 weeks before harvest. Samples (whole or part of rosette) were collected and dried in an 80° C. oven before being ground and analysed for C isotopic composition. The value used for QTL analysis for an individual line was the average of the replicated plants of that line within one run.

2. Marker Selection

The standard set of 64 markers for the Lister and Dean recombinant lines were down-loaded from the NASC website. Additional markers were added to this data set when significance was first determined to get finer scale mapping in the regions of interest. A total of 121 markers were used across the 5 chromosomes.

3. Analysis

Runs were analysed using Simple Interval Mapping (SIM) (Lander & Botstein 1989) and Composite Interval Mapping (CIM) (Zeng 1993 & 1994). Two programs were used to analyses the data, QTL Cartographer version 1.14 (Basten et al. 1999) and MQTL version 0.98 (Tinker and Mathers 1995). The two programs differ in how they deal with background markers for Composite Interval Mapping (CIM). Within MQTL the background markers are chosen at random and put into the map input file. Within QTL Cartographer the background markers are not chosen at random but rather are chosen from the Stepwise Regression analysis selecting the “best” background markers. The setting or choosing of these markers also has an influence on the level of statistical significance. Tinker argues that it is not possible to find an appropriate threshold for statistical error control when background markers are selected based on the data. Hence we used the two programs and have concentrated on QTLs that were present in both sets of analysis.

a) QTL Cartographer

Qstat was used to determine whether the data had a normal distribution (if not then measures were taken to fix the distribution). Linear Regression (LR) and Stepwise Regression (SR) were performed using the default settings (Stepwise regression used forward with backward elimination) 5% significance. Simple Interval and CIM were performed using the Zmap.qtl function. The data were analysed across all chromosomes with a walking speed of 2 cM. For model 6 (CIM) the number of background parameters was left at the default of 5 along with the window size which was left on the default of 10 cM. One thousand permutations were performed within CIM (Churchill and Doerge 1994). Eqtl was then run to determine the significant QTLs.

b) MQTL

The same set of markers used in QTL Cartographer was used in MQTL. Background markers were chosen at random for CIM. The number of markers chosen was approximately half that of the number of RILs used in the set. The default setting of a walking speed of 5 cM was selected, 3000 permutations were performed to determine significance levels with type 1 error set at 5%.

QTLs that were present in both programs and from varied background marker sets from within MQTL were considered genuine. This, coupled with repeated QTL analysis across independent experiments, lead to a significant repeatable locus surrounding the ERECTA gene on Chromosome 2 (Table 5).

Data in Table 5 indicate that there is a major QTL with a LOD score significant at the probability level of 5% and, for most runs, of even 1%, on Arabidopsis thaliana chromosome 2. In all cases, that interval sits above the ER marker on chromosome 2. Depending on the experimental run, this QTL explains 18 to 64% (see column R²) of the total genetic variance in transpiration efficiency.

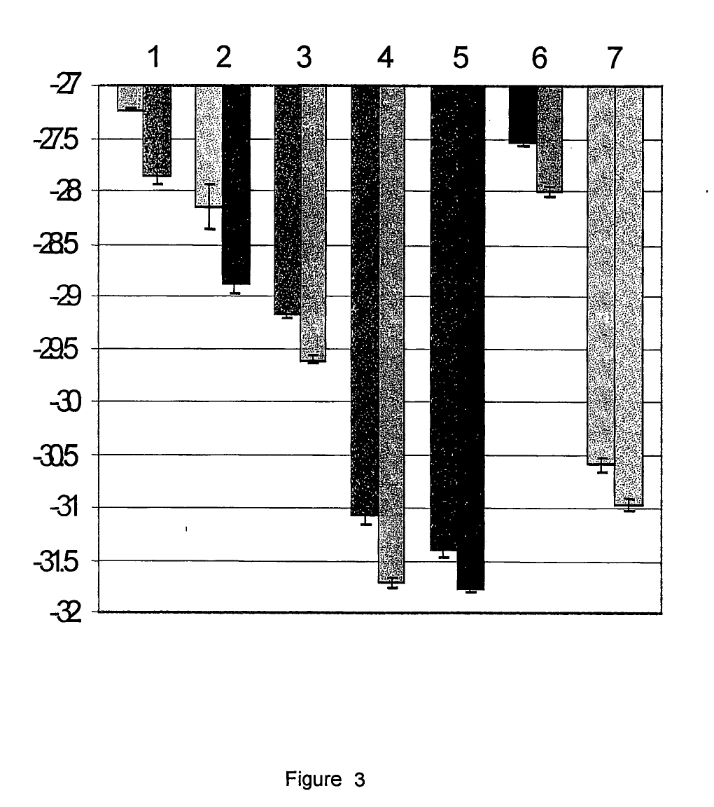

Data in FIG. 3 indicate a positive additive effect of the identified QTL based upon the mean value of the carbon isotope composition in plants carrying the Col-4 ERECTA allele.

### Example 8

### Complementation Test: Transformation of A. thaliana Lines Carrying erecta Mutations with the Wild Type ERECTA Gene Under the Control of the 35S Promoter

1. Methods

Two Columbia erecta lines were transformed using a binary vector generously given by Dr Keiko Torii. That plasmid was constructed using the vector plasmid pPZP222 (see details on this vector in Hajdukiewics et al. Plant Mol Biol 25, 989-994, 1994). The pPZP vectors carry chimeric genes in a CaMV 35S expression cassette that confer resistance to kanamycin or gentamycin in plants. The plant selectable marker (gentamycin resistance gene for the pPZP222 vector) is cloned next to the LB. Cloning sites for the gene of interest (ER in our case) is between the plant marker and the RB sequences. This ensures that that gene is transferred to plant first, followed by the gent gene. Resistance to gentamycin will therefore be obtained only if the ER gene is also present.

The binary vector was transferred to disarmed strain AGL1 of Agrobacterium tumefaciens by standard tri-parental matings (Ditta et al, 1980, PNAS 77,7347-7351) using the pRK2013 helper strain of E coli.

Arabidopsis plants were transformed using the standard floral dip method for transformation by disarmed strains of A tumefaciens (Clough and Bent, 1998, The Plant Journal 16, 735-743).

Two Columbia erecta lines were transformed, for which we had numerous data showing consistently more negative isotopic values in those lines (ie lower transpiration efficiency) than in near-isogenic Col ER wild type plants. These two lines were as follows:
1. er 105, a knock-out mutant due to the insertion of a large piece of DNA in the ERECTA gene and
2. line Col-er2 (3401 NASC identifier), same as er106 (Lease et al. 2001).

Seedlings were screened on MS plates on 100 μg/ml gentamycin sulfate. Putative transformants were transferred to soil and their progeny screened again for gentamycin resistance, for confirmation and identification of homozygous lines and T3 seed collection.

Many independent transformant lines were obtained and among those were several ER homozygous lines, which were selected for subsequent analysis (see Table 6).

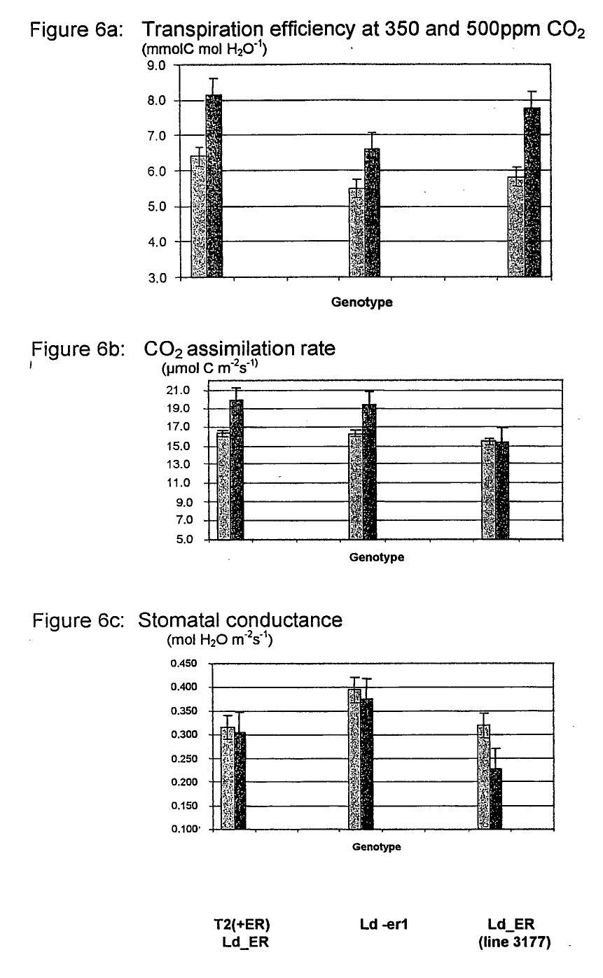

A stable transgenic homozygous Landsberg ER line also obtained by transforming the Ld-er1 ecotype (NW20) with the same construct as described above was given to us by Dr Keiko Torii (line T3-7K in Table 6 or “T2+ER” in FIGS. 6-9).

2. Results:

Initial analysis of several ER transformants in the Col-er105, Col-er106/er2, and Ld-er1 background (as shown in Table 6 above):

Effective transformation was ascertained and ER expression levels were quantified in several independent T2 transformants using real-time quantitative PCR (ABI PRISM 7700, Sequence Detection System User Bulletin #2. 1997). Basically that technique allowed us to quantify the copy number of the ER gene in lines of interest, after normalisation to the copy number of a control gene, in the same plants (same RNA pool). 18S ribosomal RNA gene was used as a control gene after checking its expression was not affected by changes in ER expression.

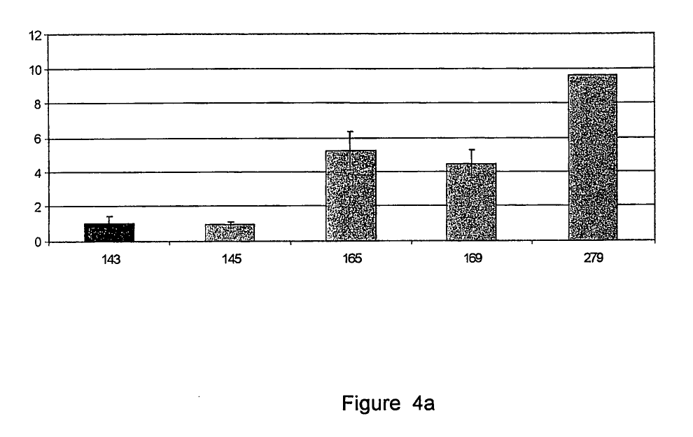

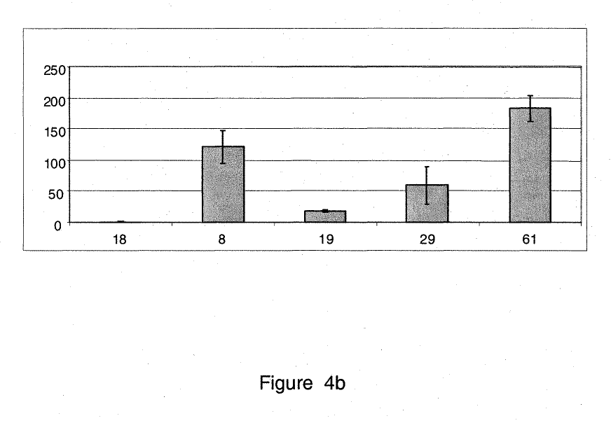

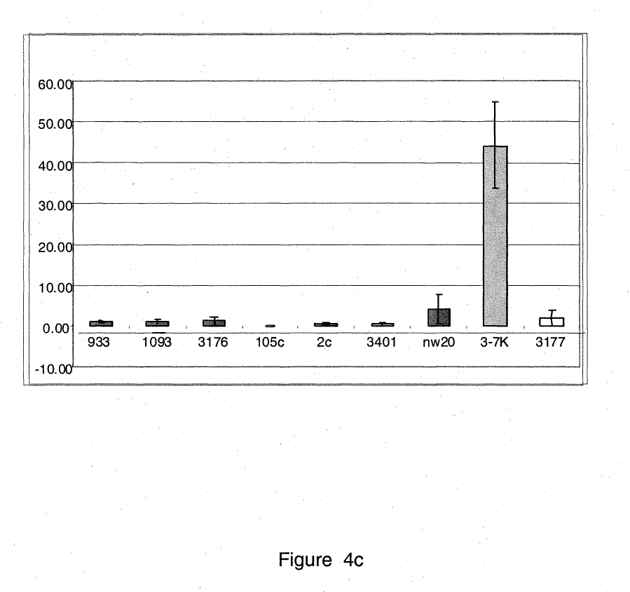

Results are shown in FIGS. 4a, 4b and 4c, wherein the y-axis in each figure describes the erecta mRNA copy number (normalised to that of 18S mRNA) in wild type ER lines, er mutants, and ER transgenics in both Columbia and Landsberg backgrounds.

All ER transgenic lines, except line 145 (FIG. 4a) showed increased mRNA copy number: from 4 to 170 fold increase compared with the null controls. Interestingly, all lines, even those with hugely increased mRNA levels look “normal”, healthy and of similar size.

Initial phenotypic analysis shows complementation of the “transpiration efficiency phenotype”. In other words, ER transgenic lines show less negative carbon isotopic composition values than null er control and null lines as shown in Table 7. Those values converge towards values measured for wild type ER ecotypes. Hence in a Columbia background, ER transgenics display values of −30.6 to −31.2 per mil on average compared to values of −31.7 to −32.2 per mil in the null transgenics (Table 7), and −30.9 per mil in the Col0 ER wild type (background ecotype for mutant er-105). The less negative carbon isotopic compositions in ER transgenics is indicative of greater transpiration efficiency in these plants, as expected.

The data presented in Table 7 are confirmed by direct measurement of leaf transpiration efficiency (ratio A/E of CO₂ assimilation rate per unit leaf area to transpiration rate) using gas exchange techniques. Stomatal density, leaf photosynthetic capacity and growth rate are also determined to analyze the underlying causes of the reversion of the transpiration efficiency phenotype (leaf development and anatomy, biochemical properties of leaves, stomatal characteristics).

### Example 9

### Tissue Specificity in the Expression of the ERECTA Gene in Wild Type Rice Oryza sativa (cv Nipponbare):

An ortholog of the A. thaliana ERECTA allele (SEQ ID NO: 1) in rice was identified in silico by homology searching of the NCBI protein database using the BLAST programme under standard conditions. The input sequence was SEQ ID NO: 2. The nucleotide sequence of the rice ortholog is presented in SEQ ID NO: 3, with the encoded protein comprising the amino acid sequence set forth in SEQ ID NO: 4.

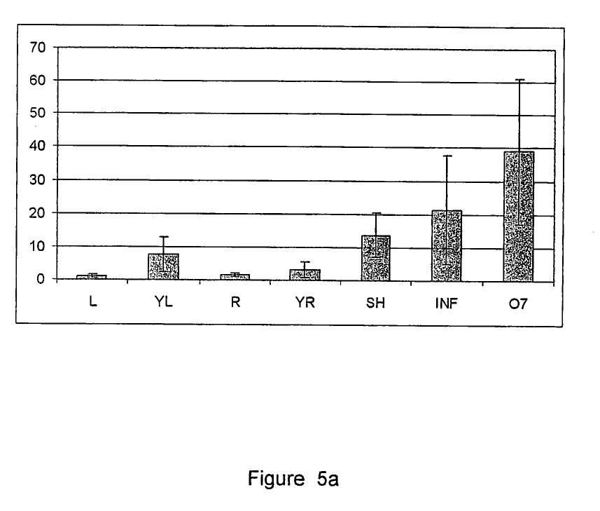

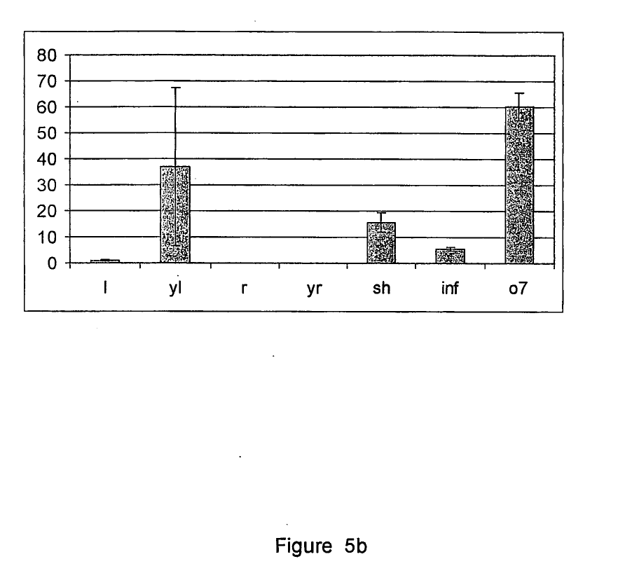

The mRNA copy number of the rice ERECTA gene was determined for various plant organs/parts, as indicated in FIGS. 5a and 5b. ERECTA mRNA copy numbers were determined by quantitative real-time PCR, using 18S mRNA as an internal control gene for normalization of data. The pattern of ERECTA expression in rice was similar to the pattern of gene expression in A. thaliana, with highest expression observed in young meristematic tissues, young leaves and even more, the inflorescences. No or very low expression is found in roots, as for A. thaliana.

These similarities in tissue specificity between rice and Arabidopsis indicates that the rice orthologue provided herein as SEQ ID NO: 3 is a true orthologue of the A. thaliana ERECTA allele set forth in SEQ ID NO: 1, with similar function

### Example 10

### Demonstration of a Functional Role for the Rice ERECTA Gene in Modulating Transpiration Efficiency

To determine a functional role for the rice ERECTA gene (SEQ ID NO: 3), lines of rice plants carrying transposon insertions that affect expression of that gene are analyzed.

Nine such mutants were identified in the publicly available collection of transposon TOS17 insertional mutants at the Japanese NIAS Institute. The TOS 17 retrotransposon is described in detail by Hirochika, Current Opinion in Plant Biology, 4, 118-122, 2001 and by Hirochika Plant Mol Biol 35, 231-240, 1997, which is incorporated herein by reference. The nine mutant lines were identified through the website URL http://tos.nias.affrc.go.jp/˜miyao/pub/tos17/, and they have the accession numbers NG0578 (mutant A), ND3052 (mutant B), ND4028 (mutant C), NC0661 (mutant D), NE1049 (mutant E), NF8517 (mutant F), NE8025 (mutant G), NE3033 (mutant H) and NF8002 (mutant I).

Nine transposon insertional mutants were ordered from NIAS, that carry the TOS17 stable retrotransposon insert in various parts of the ERECTA gene in the Nipponbare background, the genotype used for rice genome sequencing: NG0578, ND3052, ND4028, NC0661, NE1049, NF8517, NE8025, NE3033 and NF8002.

The transposon insertions in these nine lines affect the membrane spanning region of the protein (mutants I, D, E) or the Leucine Rich Repeat (LRR) domains (mutant H and G) in LRR 7 and LRR 18, respectively. In mutant B, TOS17 alters the coding sequence just upstream of sequences encoding the protein kinase domain I. In mutants C and F, the TOS17 insertion alters the sequence encoding domain VIa of the ERECTA protein. In mutant A, the TOS7 insertion is in a sequence encoding a region between domains IX and X. The sequence information on these mutants is publicly available from the NIAS website.

Using mutant seed for lines A-I received from NIAS, plants were grown for amplification of seed and analysis. Except in two mutants where several plants died, plants look healthy, with good growth indicating that, as in Arabidopsis, there is great potential to alter the ERECTA gene towards altered transpiration efficiency without adversely affecting growth and/or yield.

Based upon sequence information for each mutant A-I, primers were designed to amplify the mutant erecta alleles from seedling material derived from 20 seeds. Amplification is performed under standard conditions, to identify for each mutant, plants that are homozygous, heterozygous or null at the ERECTA locus. Homozygous TOS17 mutants B and E, and heterozygous lines and null lines in all lines A-I were identified.

In parallel to gaining information on whether or not the mutant lines were homozygous or heterozygous or null mutants, specific plant parts are removed for analysis of the consequences of the mutations on the ERECTA gene expression (levels and tissue specificity of expression), using quantitative real-time PCR as described herein. Additionally, the transpiration efficiency phenotype of each mutant line is determined by measuring C&O isotopic composition and ash contents of plant samples.

Initial results on ¹³C isotopic composition of mature blades of rice seedlings reveals significant variation between mutant lines (−32.8 to −34.2 per mil) and, in at least 4 mutants, significant deviations from the wild type values, towards more negative values, suggesting that the erecta mutations do affect transpiration efficiency in rice, as in Arabidopsis.

Similar methods as above are applied to anaylzing the progeny of the mutant plants, to facilitate analysis of the effects of the erecta mutations under a range of conditions, including flooding (as is the most common practice for Nipponbare), water stress such as from soil drying (upland rice growth conditions) or low air humidity (heat spells). Differences in plant morphology, anatomy and apical dominance are noted under each environmental condition. Parameters that are characterised include tillering patterns, the anatomy of leaves and meristems, development and growth rates.

Comparisons between mutants A-I are further used to characterize the role of the different protein domains in conferring different phenotypes observed for each line under different environmental and/or agricultural growth conditions. It is interesting that, among the 4 mutants that exhibit much lower C isotopic composition than the wild type, three are those mutants where the TOS17 insert affects the membrane spanning region.

### Example 11

### Effect of Silencing ERECTA Gene Expression on Transpiration Efficiency

To confirm the role of the ERECTA gene in conferring the transpiration efficiency phenotype on a plant, expression of the wild-type ERECTA allele is reduced or inhibited using standard procedures in plant molecular biology, such as, for example, antisense inhibition of ERECTA expression, or the expression of inhibitory interfering RNA (RNAi) that targets ERECTA expression at the RNA level. All such procedures will be readily carried out by the skilled artisan using the disclosed nucleotide sequences of the ERECTA genes provided herein or sequences complementary thereto.

For transformation of rice and Arabidopsis, transgenes are prepared in disarmed, non-tumorigenic binary vectors carrying T-DNA left and right borders and a selectable marker operable in E Coli.

Binary vectors used for DNA transfer include vectors selected from the group consisting of:
1. pPZP222 (Hajdukiewicz et al, 1994, Plant Mol Biol 25, 989-994);
2. PBI 121 (Clonetech) (Ueda et al 1999, Protoplasma 206, 201-206);
3. pOCA18 (Olszewski et al 1988, Nucl. Acid Res, 16 10765-10782);
4. pGreen and pSoup or variants thereof (Hellens et al., 2000, Plant Mol Biol 42, 819-832) and
5. binary vectors developed on the pCAMBIA vectors backbone described at the webiste of CAMBIA.

The starting material for all these vectors was the backbone developed by Hajdukiewicz et al., 1994. The pPZP series of vectors comprise (i) a wide-host-range origin of replication from the Pseudomonas plasmid pVS1, which is stable in the absence of selection; (ii) the pBR322 origin of replication (pMB9-type) to allow high-yielding DNA preparations in E. coli; (iii) T-DNA left (LB) and right (RB) borders, including overdrive; and (iv) a CaMV35S promoter expression cassette. While the pPZP series of vectors also served as the backbones for the pCAMBIA series, they have been very extensively modified for particular applications.

Vectors containing in their T-DNA various combinations of the following components are particularly preferred:
1. hptII resistance gene cassette for conferring resistance to hygromycin on transformed plant material, wherein expression of hptII is operably under control of Ubil or 35S promoter;
2. a reporter gene cassette comprising nucleic acid encoding the EGFP (Enhanced Green Fluorescence Protein) and/or beta-glucuronidase (GUS and GUSPlus) reporters;
3. Gal4/VP16 transactivator cassette; and
4. one or more plant gene expression cassettes comprising either full-length or partial cDNAs of ERECTA genes in the sense or antisense orientation, or capable of expressing RNAi comprising sequences derived from the ERECTA gene, including any genomic fragments of plant DNA.

The binary vectors are transferred to disarmed strain AGL1 of Agrobacterium tumefaciens by standard tri-parental matings (Ditta et al, 1980, Proc. Natl Acad. Sci. 77,7347-7351) using the pRK2013 helper strain of E coli. A thaliana plants are transformed using the standard floral dip method for transformation by disarmed strains of A tumefaciens (Clough and Bent, 1998, The Plant Journal 16, 735-743). Rice is transformed by generating embryogenic calli from excised embryos and subjecting the embryogenic calli to Agrobacterium tumefaciens mediated transformation according to published procedures (eg Wang et al 1997, J Gen and Breed, 51 325-334, 1997).

Transformed plants are analyzed to confirm that those lines expressing antisense or RNAi constructs have reduced expression of functional ERECTA protein and more closely resemble the erecta phenotype than do wild-type plants or plants ectopically expressing a wild-type ERECTA gene in the sense orientation.

### Example 12

### Identification of a Sorghum Ortholog of A. thaliana ERECTA

An ortholog of the A. thaliana ERECTA allele (SEQ ID NO: 1) in sorghum was identified in silico by homology searching of the NCBI protein database using the BLAST programme under standard conditions. The input sequence was SEQ ID NO: 2. The nucleotide sequence of the sorghum ortholog is presented in SEQ ID NO: 5, with the encoded protein comprising the amino acid sequence set forth in SEQ ID NO: 6.

### Example 13

### Identification of A. thaliana ERECTA Homologs

Two homologs of the A. thaliana ERECTA allele (SEQ ID NO: 1) were identified in silico by homology searching of the NCBI protein database using the BLAST programme under standard conditions. The input sequence was SEQ ID NO: 2. The nucleotide sequences of the A. thaliana ERECTA homologs are presented in SEQ ID NOs: 7 and 9, with the encoded proteins comprising the amino acid sequences set forth in SEQ ID NOs: 8 and 10, respectively.

T-DNA insertional mutants for these two homologous genes, both on chr5, have been identified in the Salk Institute mutant collection (web address: signal.salk.edu/cg:-bin/tdnaexpress). Several of these mutants were ordered: Salk_{—}007643 and Salk_{—}026292 for gene At5g07180; Salk_{—}045045 and Salk_{—}081669 for gene At5g62230. Primer pairs were designed in order to determine insert copy number and homozygozity/heterozygozity in the seedlings grown from the seeds that were received. Homozygous lines with 1 insert were identified and are under characterisation in order to compare the expression patterns (tissue localisation and mRNA levels) of the two genes and of the ERECTA gene across a range of environmental conditions and determine whether the three genes are functionally related.

### Example 14

### Identification of Wheat Orthologs of A. thaliana ERECTA

Partial cDNA sequence of orthologs of the A. thaliana ERECTA allele (SEQ ID NO: 1) in wheat were initially identified in silico by homology searching of the NCBI protein database using the BLAST programme under standard conditions. It was necessary, however, to conduct additional searches of private databases in order to link the partial sequences identified in the NCBI database. Correction of partial sequences located in the NCBI database was also necessary in order to generate a contig corresponding to the wheat ERECTA ortholog.

The input sequence is the A. thaliana (SEQ ID NO: 2) or rice (SEQ ID NO: 4) amino acid sequences or a nucleotide sequence encoding same. The nucleotide sequences of the wheat ortholog are presented in SEQ ID NOs: 11-19, with the encoded proteins comprising the amino acid sequences set forth in SEQ ID NO: 20.

The sequence set forth in SEQ ID NOs: 11 to 18 are partial cDNA sequences. The corresponding sequence of the wheat ERECTA ortholog (SEQ ID NO: 19) is isolated by standard nucleic acid hybridization screening of a wheat cDNA library.

To confirm the role of the wheat ERECTA orthologs in transpiration efficiency, expression data sets are used for in silico studies of ERECTA gene expression in a range of tissues of wheat plants grown under a range of environmental conditions, thereby providing indications of tissue specificities in expression patterns and preliminary data on the types of environments where the ERECTA ortholog is most likely to play a physiological role in relation to water use in this species. In these studies, nucleic acid comprising the sequence set forth in SEQ ID NO: 11 to 19, or a sequence complementary thereto, are used to produce hybridization probes and/or amplification primers.

Additionally, an ERECTA gene (SEQ ID NO: 11 to 19) in the sense or antisense orientation is introduced into wheat, thereby producing transformed expression lines. Gene constructs are specifically to silence ERECTA gene expression using RNAi technology, or alternatively, to ectopically express the entire open reading frame of the gene.

Based upon similar function, the open reading frame of the A. thaliana ERECTA gene (i.e., SEQ ID NO: 1) is also introduced into wheat plant material in the sense orientation, thereby ectopically expressing A. thaliana ERECTA in wheat.

Gene constructs are introduced into wheat following any one of a number of standard procedures, such as, for example, using A. tumefaciens mediated transformation as described in published AU 738153 or EP 856,060-A1 or CA 2,230,216 to Monsanto Company, or using published biolistic transformation methods as described by Pellegrineschi et al., Genome 45(2), 421-30, 2002. Accordingly, genetic transformation is readily used to generate wheat lines with altered expression of an ERECTA gene. About 30 to 40 different transformants are produced, depending upon the efficiency of RNAi in reducing expression of ERECTA in wheat.

Primary transformants (T0) are characterized to determine the number and loci at which transgenes are inserted. T1 and T2 segregating progenies are then generated from selected T0 transformants, and analyzed to determine segregation ratio and to confirm the number of loci having inserted transgenes. Those T1 and/or T2 lines having single transgene insertions are selected and used to generate and multiply seed for physiological studies.

Water use efficiency in the T1 and/or T2 lines is determined through (a) gravimetric measurements of water transpired and biomass increases; (b) ¹³C isotopic discrimination in plant tissues, (i.e., by determining Δ; and (c) ash content of plant tissue.

Meristem and leaf development are also analyzed, especially with respect to the differentiation and anatomy of the epidermis, the stomatal complexes and the mesophyll tissue and by examining leaf gas exchange properties. This is done using microscopy, in situ imaging techniques and concurrent on-line measurements of C isotopic discrimination (Δ) and of CO₂ and water fluxes in and out of leaves. Information on gene regulation and the network of genes in which the ERECTA ortholog operates in its effects on transpiration efficiency, is determined by transcriptome analysis of a restricted set of the transgenic lines with altered ERECTA expression.

As described herein for A. thaliana and rice, correlations between physiological measurements and gene expression level or copy number confirm the role of the ortholog in conferring the transpiration efficiency phenotype in wheat.

### Example 15

### Identification of a Maize Ortholog of A. thaliana ERECTA

Partial cDNA sequence of ortholog of the A. thaliana ERECTA allele (SEQ ID NO: 1) in maize were initially identified in silico by homology searching of the NCBI protein database using the BLAST programme under standard conditions. It was necessary, however, to conduct additional searches of private databases in order to link the partial sequences identified in the NCBI database. Correction of partial sequences located in the NCBI database was also necessary in order to generate a contig corresponding to the maize ERECTA ortholog.

The input sequence was SEQ ID NO: 2. The nucleotide sequence of a maize ortholog is presented in SEQ ID NOs: 21 to 44, with the encoded protein comprising the amino acid sequence set forth in SEQ ID NO: 45.

The sequence set forth in SEQ ID NOs: 21 to 43 are partial cDNA sequences. The corresponding sequence of the maize ortholog (SEQ ID NO: 44) is isolated by standard nucleic acid hybridization screening of a wheat cDNA library.

To confirm the role of the maize ERECTA ortholog in transpiration efficiency, expression data sets are used for in silico studies of ERECTA gene expression in a range of tissues of maize plants grown under a range of environmental conditions, thereby providing indications of tissue specificities in expression patterns and preliminary data on the types of environments where the ERECTA ortholog is most likely to play a physiological role in relation to water use in this species. In these studies, nucleic acid comprising the sequence set forth in SEQ ID NO: 15, or a sequence complementary thereto, is used to produce hybridization probes and/or amplification primers.

Additionally, collections of transposon-tagged maize mutants are searched to select those having insertions that affect expression of the ERECTA gene and the expression level and/or copy number of the ERECTA ortholog is correlated to transpiration efficiency under the range of environmental growth conditions, essentially as described herein for A. thaliana and rice.

Additionally, an ERECTA gene in the sense or antisense orientation is introduced into maize, thereby producing transformed expression lines. Gene constructs are specifically to silence ERECTA gene expression using RNAi technology, or alternatively, to ectopically express the entire open reading frame of the gene.

Based upon similar function, the open reading frame of the A. thaliana ERECTA gene (i.e., SEQ ID NO: 1) is also introduced into maize plant material in the sense orientation, thereby ectopically expressing A. thaliana ERECTA in maize.

Gene constructs are introduced into maize following any one of a number of standard procedures, such as, for example, any of the methods described by Gordon-Kamm et al., Plant Cell 2(7), 603-618, 1990; U.S. Pat. No. 5,177,010 to University of Toledo; U.S. Pat. No. 5,981,840 to Pioneer Hi-Bred; or published US application No. 20020002711 A1 (Goldman and Graves);. Accordingly, genetic transformation is used to generate maize lines with altered expression of an ERECTA gene.

About 30 to 40 different transformants are produced, depending upon the efficiency of RNAi in reducing expression of ERECTA.

Primary transformants (T0) are characterized to determine the number and loci at which transgenes are inserted. T1 and T2 segregating progenies are then generated from selected T0 transformants, and analyzed to determine segregation ratio and to confirm of number of loci having inserted transgenes. Those T1 and/or T2 lines having single transgene insertions are selected and used to generate and multiply seed for physiological studies.

Water use efficiency in the T1 and/or T2 lines is determined through (a) gravimetric measurements of water transpired and biomass increases; (b) ¹³C isotopic discrimination in plant tissues, (i.e., by determining Δ); and (c) ash content of plant tissue.

Meristem and leaf development are also analyzed, especially with respect to the differentiation and anatomy of the epidermis, the stomatal complexes and the mesophyll tissue and by examining leaf gas exchange properties. This is done using microscopy, in situ imaging techniques and concurrent on-line measurements of Δ and of CO₂ and water fluxes in and out of leaves. Information on gene regulation and the network of genes in which the ERECTA ortholog operates in its effects on transpiration efficiency, is determined by transcriptome analysis of a restricted set of the transgenic lines with altered ERECTA expression.

As described herein for A. thaliana and rice, correlations between physiological measurements and gene expression level or copy number confirm the role of the ortholog in conferring the transpiration efficiency phenotype in maize.

### Example 16

### Mechanism of Enhanced Transpiration Efficiency and Inheritance of ERECTA in Arabidopsis (Landsberg and Columbia Backgrounds)

The present inventors performed direct measurements of transpiration efficiency (ratio of CO₂ assimilation rate to transpiration rate) in both Landsberg and Columbia backgrounds. To confirm the role of ERECTA under a wider range of agronomically relevant conditions, the transpiration efficiencies of transformed plants carrying an ERECTA allele in response to varying environmental conditions (i.e., soil water and ion content, atmospheric humidity and CO₂ levels) were determined and compared to the response of wild type plants (e.g., ER). Results of these experiments are presented in FIGS. 6-11, and Tables 8 and 9.

Data in FIG. 6 show that the enhanced transpiration efficiency obtained by inserting a transgene carrying the wild type ER allele in the Ld-er1 mutant (line T2+ER) is mostly due to a decreased stomatal conductance. The phenotype of the transgenic line (T2+ER in graphs) is similar to that of a Ld-ER ecotype near isogenic to Ld-er1 obtained from the Stock Centre (line 3177 on graphs). The increased transpiration efficiency in transgenic ER, compared to levels observed in wild type ER line is observed under both current ambient CO₂ levels and increased CO₂ levels that are within the limits predicted to occur worldwide over the next two decades.

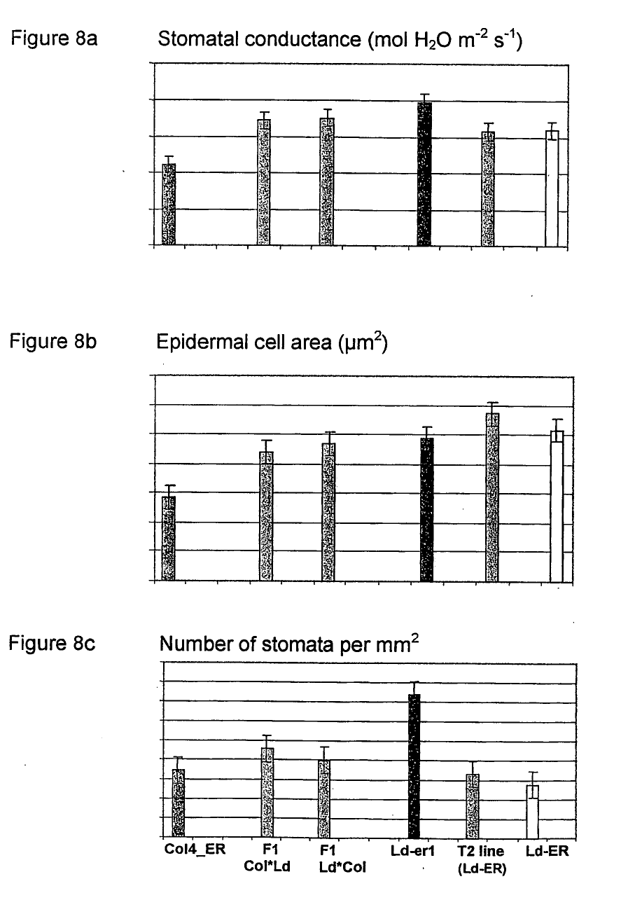

Data in FIG. 8 show that the reduced stomatal conductance in the ER T2 transgenic line compared to the Ld-er1 line is, at least for a large part, caused by a reduced stomatal density (decrease in the number of stomata per unit area by more than half, down to similar levels as those observed in wild type Ld-ER). This decrease in stomatal density is relatively higher than that in the density of epidermal cells whose surface area is increased by only about 10%. It therefore follows that the ER transgene has affected stomatal development, specifically, and caused a decreased in stomatal index. These data show complementation with respect to the processes driving variation in transpiration efficiency.

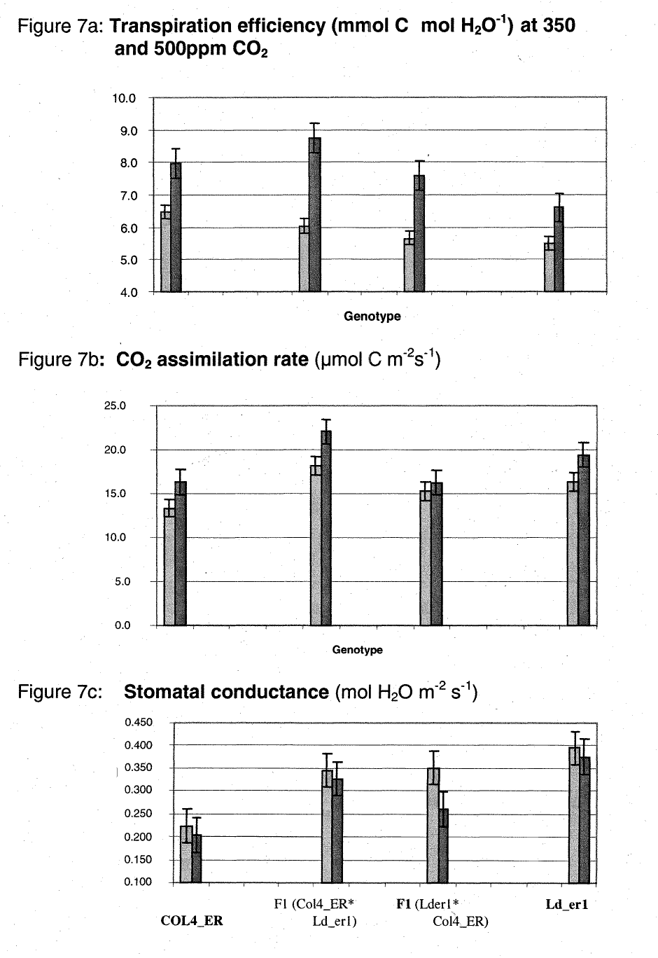

Reciprocal crosses were also performed between the two parental lines NW20 (Ld-er1) and Col4 (Col-ER). The notation F1 (Col*Ld) refers to the F1 plants where Col was the recipient of Ld pollen, while the notation F1 (Ld*Col) indicates the converse (Ld ovary receiving Col pollen). Initial analysis of these two types of F1 plants has been made for: gas exchange and photosynthetic properties, transpiration efficiency (FIG. 7) and C isotopic composition (Table 9), rosette shape and developmental rate, anatomy of leaf epidermis (FIG. 8), flowering date, inflorescence and pod shape. Consistent with our analysis of complementation experiments, the data show that the ERECTA gene affects all these phenotypes and not only inflorescence and pod shape.

The data also show a complex inheritance of the ERECTA gene, such that the gene is dominant, with no reciprocal effect on pod shape (longer pods, longer stems and pedicels in all F₁ plants, similar to the Col-ER parent). However, for other traits, results indicate maternal effects: hence the transpiration efficiency values (see FIG. 7a) and rosettes carbon isotope composition in F₁ plants (Table 9) are intermediate between the parental values, but different between the two sets of F1 plants: values for F₁ plants (Col*Ld) are closer to the Col values, while those for F1 plants (Ld*Col) are closer to values for the Ld parent.

Data in FIG. 8 indicate that stomatal conductance (transpiration per unit leaf area, FIG. 8a) displays values close to the Ld-er1 parent in all F1 plants, despite the stomatal densities being close to the Col-ER parent (FIG. 8c). This shows that the ER gene affects not only epidermis development but also stomatal aperture (dynamics of stomata) and that while the ER effect on stomatal density appears to be dominant, effect on stomatal aperture is not.

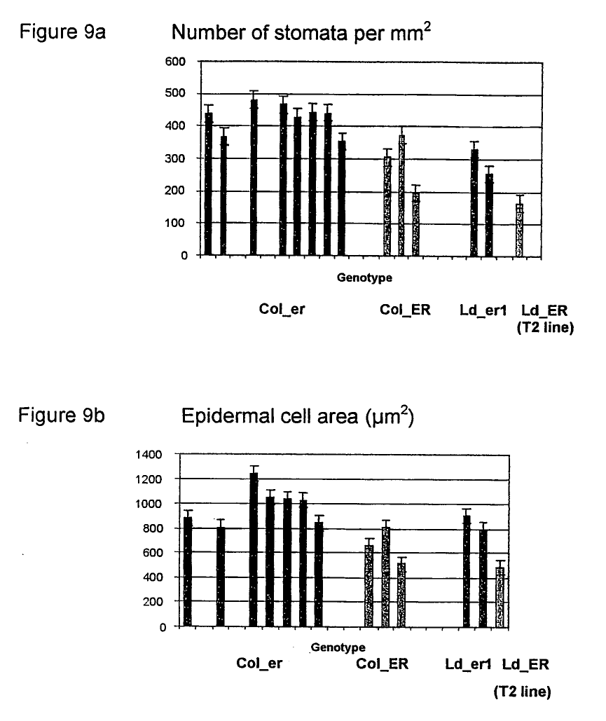

Data in FIG. 9 show the effect of various er mutations (in Col background, mutants obtained from the Stock Centre or Dr Torii) on the number of stomata per unit leaf area. The stomatal densities for all but two of those mutants are greater than those the ColER wild type leaves, and confirm the effect of the ERECTA gene on that parameter.

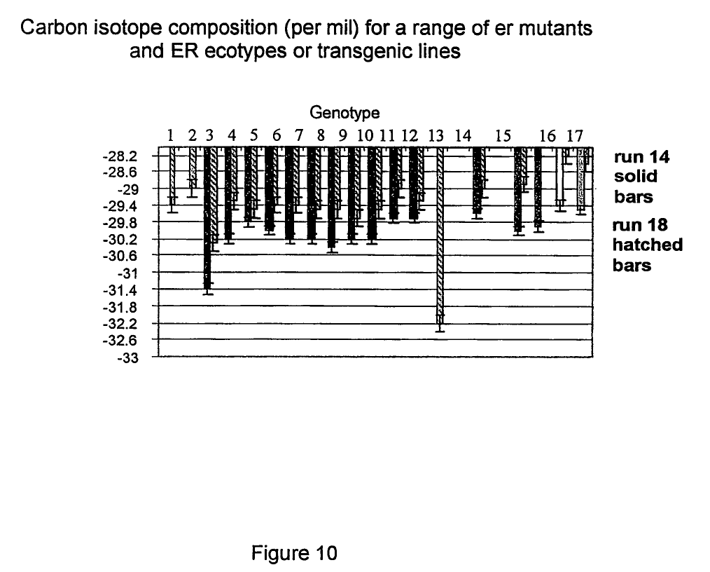

Data in FIG. 10 show that enhanced transpiration efficiency in the ER transgenic line compared with null Ld-er1 (no insertion of transgene) is confirmed by the less negative C isotopic composition values measured in leaf material (compare values for lines NW20 and CS20 (Ld er1; lines 16 and 17 on x-axis) and a transgenic T2 Id-ER line, homozygous for he ER transgene (line 19 in the Figure). The C isotopic values measured in the ER transgenic line are similar to those in the near isogenic Ld-ER ecotype (line 18 in FIG. 10). This demonstrates complementation on this phenotypic trait, and validates once again the use of C isotopic composition as a quantitative indicator (substitute) of transpiration efficiency.

Data in FIG. 10 also the C isotopic compositions of a range of Col-er mutants, including those analysed in FIG. 9 for stomatal densities. Most mutants show more negative C isotopic values than the COL-ER ecotype. This is consistent with the increased stoamtal densities described in FIG. 9 and with all other comparisons of C isotopic compositions or direct measurements of transpiration efficiencies in er/ER lines and again indicative of the positive effect of the ER allele on transpiration efficiency.

A few mutants in FIG. 10 stand out, eg Col-er105, or line 3140 (a line from NASC carrying the er1 and gl1-1 mutations). As genetic information is available for these mutants (nature and position of mutations) these mutants provide very useful functional information on the protein domain(s) of the ERECTA protein that are essential for conferring the transpiration efficiency phenotype and underlying processes.

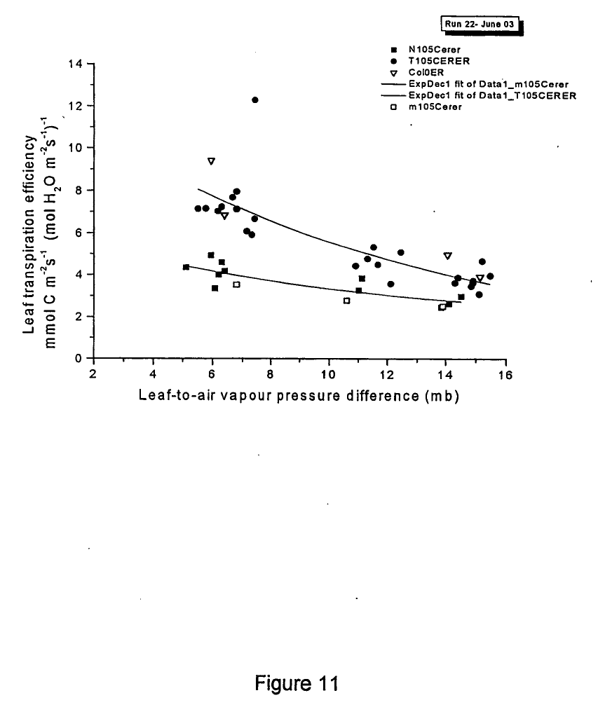

The present inventors also perform direct measurements of transpiration efficiency (ratio of CO₂ assimilation rate to transpiration rate) in several T2 transformants generated in a Columbia background (i.e. transformation of mutant er-105 and er-2/106 above). Results from these measurements are shown in FIG. 11. These data show that the phenotype can be complemented in a Columbia background, as determined by measuring transpiration efficiency, transpiration and CO₂ assimilation rates. Complementation is observed under conditions of both high humidity and low humidity, hence the demonstration that the ERECTA gene plays a role in the control of transpiration efficiency under both well watered and drought conditions, and that overexpression of that gene has the potential of increasing growth and resistance to drought and drought related stresses.

More particularly, the data in FIG. 11 demonstrate the role of the ERECTA gene on transpiration efficiency across a range of humidities, including low humidities such as prevail in warm and dry areas:
the er-105 mutant which carries a knock-out mutation of ERECTA (quasi no ER transcript) (open black squares) in Col0 background has lower transpiration efficiency than the wild type near isogenic Col0 (open triangles).
this mutant was transformed with an ER transgene under the 35S promoter and several ER homozygous T2 lines were produced (solid circles). Those lines (5 independent transformants are included in the graph) have much increased transpiration efficiencies (+40 to 70%) compared to the null lines (solid squares) and similar to those measured for the wild type ER-Col0 line, across the whole range of leaf to air vapour pressure deficit tested In our experiments.

Additionally, null lines that carry no transgene insertion but went through transformation and selection on antibiotics display similar values as the starting er-105 mutant demonstrating that these manipulations themselves have no detectable confounding effect on transpiration efficiency.

TABLE 1
QTL Analysis of Carbon Isotope Discrimination in Lister and Dean's Recombinant Inbred Lines

| RUN No. | chr2 locus | QTL | chr4 locus | CONCLUSION | | |
|---|---|---|---|---|---|---|
| Experimental conditions | (cM) | analysis method | (cM) | QTLs number | predicted map position | |
| Run 1 (40 lines) Glasshouse- | | | | | | |
| 12 h day length | 58.5 | SIM&CIM | | 2 | chr2: | 58.5-61.02 |
| irradiance150-350 μE m⁻² s⁻¹ | 46.77 | SIM&CIM | | | chr2: | 46.77-50.75 |
| Seedlings transferred from | 61.02 | SIM | 108.5 | | | |
| agar plates | | | | | | |
| Run1 data | | | | | | |
| but with using | | | | | | |
| different markers | | | | | | |
| | 56.94 to 58.00 | CIM&SIM | | 1 | | |
| | 46.77 to 50.75 | SIM | | | | |
| | 63.02 | | | | | |
| Run1 | | | | | | |
| with different | | | | | | |
| number of lines | | | | | | |
| | 58.5 to 61.02 | | | 2 | | |
| | 56-61 | | | | | |
| Run2 | | | | | | |
| Glasshouse September | | | | | | |
| from seeds sown on soil | | | | | | |
| batch 1 | 50.75 | CIM (QTL cart) | | 2 | chr2: | 56.94-61.02 |
| | 61.02 | MQTL | | | chr2: | 50.75 |
| batch 2 | ?50.75 | MQTL | | NS | | |
| batch3-5 | | | | | | |
| all batches | 58.5 | MQTLcart | | NS | | |
| | 56.94-58.5 | MQTL | | | | |
| Run 3 | | | | | | |
| 37 lines: parents and lines with | | | | | | |
| crossing-overs on chromosome 2 | | | | | | |
| 5 growth conditions differing | | | | | | |
| in humidity, irradiance, mode | | | | | | |
| of establishment (seeds sown | | | | | | |
| on soil or seedlings | | | | | | |
| transplanted from agar) | | | | | | |
| batch B | 61.02-61.06 | | 108 NS | | | |
| batch C | 56.94-58.00 | | | | | |
| batch D | 63.02 | QTLcar | | | | |
| | 63.02 | MQTL | | | | |
| all batches (conditions) | 58.5 | | | 1or 2 | chr2: | 56.94-58.5 |
| | 61.02 | | | 3 | chr2: | 61.02-63.02 |
| Run 4 | | | | | | |
| same lines as Run 3 growth | | | | | | |
| chambers 10 h daylight | | | | | | |
| | 50.74 | | | | chr2: | 50.74 |
| Run 5 | | | | | | |
| repeat of run 1 BUT | | | | | | |
| ALL lines | | | | | | |
| | 50.74 | | | 1 | chr2: | 50.74 |
| Run 7 | | | | | | |
| same lines as in run 1 but | | | | | | |
| in growth chamber | | | | | | |
| and higher light | | | | | | |
| 10 h daylength | 46.77-50.75 | CIM&SIM | | 1 | chr2: | 46.77-5065 |
| 470-510 μE m⁻² s⁻¹ irradiance | | | | | | |

TABLE 2

| | | | Isogenic ER line and |
|---|---|---|---|
| Background | Mutation | Stock Centre name | Stock Centre Name |
| Landsberg | er1 | CS20 or NW20^{a} | 3177 or CS163 |
| Columbia | er2^{b}/er106 | 3401 | Col1 or 3176 |
| Columbia | er105^{c} | | Col3 with gl1 marker |
| | | | or Col0 |

^{a}NW20 is an Ler parent for Lister and Dean's recombinant lines, carrying the er1 mutation. Lines 3177 or CS163 are the closest isogenic ER lines.
^{b}er2 is an er allele identified by Rédéi in Col background. Col1 or 3176 are the closest Col near-isogenic lines. The er2 is same mutation as mutation er-106 later reported by Torii and collaborators (Lease et al. 2001)
^{c}er105 was isolated from a fast-neutron-irradiated Col seed population (Torii et al., 1996).
^{d}Col4, the Col parent for the Lister and Dean's parent was systamically included in all comparisons.

TABLE 3
Comparison of er/ER lines in both Col and Ld background for carbon isotope discrimination
values (per mil) in leaf material under a range of environmental conditions

| | Differences in mean carbon isotope discrimination values (per mil) | | | | | | | | |
|---|---|---|---|---|---|---|---|---|---|
| | | | | | | | (7) | | |
| | (1) | | | | | | er1-Col4 | | |
| Run | er-ER | (2) | (3) | (4) | (5) | (6) | (parental lines | (8) | (9) |
| No. | (all lines) | er105-Col0 | er105-Col4 | er105-3176 | er2-3176 | er1-3177 | for RILs) | er105-Coli | er1-Coli |
| 1 | 0.13 | 0.16 | | | | | | 0.16 | |
| 2 | 0.89 | 1.18 | | | | | | 1.18 | |
| 3 | 0.26 | 0.11 | | | | | | 0.11 | |
| 4 | 1.12 | 1.60 | | | | | | 1.60 | |
| 5 | 1.03 | | 1.83 | 1.67 | 0.92 | 0.64 | 0.82 | 1.75 | 0.73 |
| 6 | 0.70 | 1.13 | 1.01 | 0.71 | 0.27 | 0.74 | 0.73 | 0.95 | 0.73 |
| 7 | 0.70 | 1.32 | 1.12 | 1.23 | 0.75 | 0.35 | 0.05 | 1.22 | 0.15 |
| 8 | 0.59 | 1.16 | 1.11 | 1.19 | 0.54 | 0.28 | 0.06 | 1.16 | 0.17 |
| 9 | 0.30 | 1.09 | 0.77 | 0.77 | 0.02 | 0.00 | 0.56 | 0.88 | 0.28 |
| 10 | 0.56 | 1.05 | 0.94 | 0.87 | 0.38 | 0.39 | 0.33 | 0.95 | 0.36 |

| | (1) | | | | | | | | |
|---|---|---|---|---|---|---|---|---|---|
| | difference | | | | | | | | |
| Run | er-ER | (2) | (3) | (4) | (5) | (6) | (7) | (8) | (9) |
| # | (all lines) | er105-Col0 | er105-Col4 | er105-3176 | er2-3176 | er1-3177 | er1-Col4 | er105-Coli | er1-Coli |
| 11 | 0.48 | | 0.40 | | | | 0.52 | 0.40 | 0.52 |
| 12 | 0.36 | 0.82 | 1.31 | 1.08 | 0.33 | 0.05 | 0.07 | 1.07 | 0.01 |
| 13 | 0.38 | | 0.90 | 0.82 | 0.07 | 0.60 | 0.52 | 0.86 | 0.56 |
| 14 | 0.65 | 1.42 | 0.60 | | | 0.58 | 0.06 | 1.01 | 0.32 |
| 15 | 0.82 | | | | | | 0.82 | | 0.82 |

For all runs:

| | | | | | | | | | |
|---|---|---|---|---|---|---|---|---|---|
| Mean | 0.60 | 1.01 | 1.00 | 1.04 | 0.41 | 0.40 | 0.41 | 0.95 | 0.42 |
| S.E. | 0.07 | 0.14 | 0.11 | 0.11 | 0.10 | 0.08 | 0.09 | 0.12 | 0.08 |

For Common runs:

| | | | | | | | | | |
|---|---|---|---|---|---|---|---|---|---|
| Mean: | 0.58 | 1.10 | 1.12 | 1.04 | 0.41 | 0.38 | 0.39 | 1.11 | 0.37 |
| S.E. | 0.08 | 0.05 | 0.11 | 0.11 | 0.10 | 0.09 | 0.10 | 0.10 | 0.09 |

TABLE 4
Run 9- December 2001: Leaf gas exchange measurements in er/ER Arabidopsis lines

| | | | | (1) | | | (4) | | | | |
|---|---|---|---|---|---|---|---|---|---|---|---|
| | | | | E | (2) | (3) | A/E | (5) | (6) | | |
| | | | | (mmol | A | Gw | (mmolC/ | pa | pi | (7) | (8) |
| | | Genotype | | H₂O/m²/s) | (μmolC/m²/s) | (mol/m²/s) | molH₂O) | (μbar) | (μbar) | pi/pa | 1-pi/pa |
| Row (1) | Ld-ER | 3177 | Mean | 3.38 | 12.33 | 0.273 | 3.67 | 360 | 282 | 0.782 | 0.218 |
| | | | S.E. | 0.48 | 1.64 | 0.039 | 0.14 | 10 | 11 | 0.010 | 0.010 |
| Row (2) | Ld-er | NW20 | Mean | 2.59 | 8.73 | 0.218 | 3.38 | 348 | 280 | 0.804 | 0.196 |
| | | | S.E. | 0.07 | 0.31 | 0.005 | 0.04 | 5 | 4 | 0.002 | 0.002 |
| Row (3) | Col-ER | 933 | Mean | 3.41 | 13.55 | 0.291 | 4.06 | 350 | 270 | 0.772 | 0.228 |
| | | | S.E. | 0.40 | 1.16 | 0.040 | 0.22 | 4 | 7 | 0.020 | 0.020 |
| Row (4) | Col-ER | 3176 (Col1) | Mean | 2.23 | 10.13 | 0.180 | 4.55 | 346 | 254 | 0.734 | 0.266 |
| | | | S.E. | 0.50 | 1.47 | 0.048 | 0.24 | 5 | 9 | 0.021 | 0.021 |
| Row (5) | Col-er | er105 | Mean | 2.27 | 8.55 | 0.198 | 3.76 | 356 | 283 | 0.795 | 0.205 |
| | | | S.E. | 0.03 | 0.17 | 0.005 | 0.07 | 11 | 10 | 0.006 | 0.006 |
| Row (6) | Col-er | er2 | Mean | 3.06 | 11.90 | 0.256 | 3.92 | 357 | 279 | 0.780 | 0.220 |
| | | | S.E. | 0.22 | 0.56 | 0.027 | 0.12 | 1 | 6 | 0.014 | 0.014 |

CONCLUSION:
ComparisonLd-ER/Ld-er er line has lower A/E with lower g and lower A. The difference in A/E is driven by A
Comparison 933/NW20 NSW20 (er1) has lower A/E with lower g and lower A
Comparison Col1/Ld-er1 The difference in A/E is driven by A
Comparison Col1/Col-er105 er105 has MUCH lower A/E with Higher g and lower A i.e. the difference in A/E is driven by A and g
Comparison Col1/Col-er2 er2 has lower A/E with MUCH higher g and HIGHER A i.e. the difference in A/E is driven by g and is opposed or not driven by A
NOTE:
pₐ and p₁ are the ambient and intercellular partial pressures or CO₂, respectively.

TABLE 5

| experimental | | | | QTL position | | | |
|---|---|---|---|---|---|---|---|
| run | LOD | P(0.05) | P(0.01) | cM gap | LOD-LOD_{P0.05} | R² | Total R² |
| 1 | 5.2861 | 4.7469 | 5.9923 | 39.32-50.65 | 0.5392 | 0.19 | 0.89 |
| 4 | 3.519 | 3.3561 | 4.2134 | 50.63-50.65 | 0.1629 | 0.29 | 0.57 |
| 7 | 9.6489 | 3.9627 | 4.9083 | 50.63-50.65 | 5.6862 | 0.53 | 0.77 |
| 9 | 6.1748 | 4.2328 | 5.0278 | 46.77-50.65 | 1.942 | 0.44 | 0.81 |
| 10 | Zero Qtl's | 3.6051 | 4.3889 | | | | |
| 16 | 11.5132 | 3.188 | 3.8896 | 48.96-50.65 | 8.3252 | 0.64 | 0.65 |
| 2(batch1) | 5.5459 | 3.3264 | 4.2907 | 50.63-51.02 | 2.2195 | 0.26 | 0.40 |

LOD = log₁₀ of the likelihood ratio

TABLE 6
Summary table of the lines used for initial functional
characterisation and analysis of ER effects:

| Background | Stable T2 homozygous | |
|---|---|---|
| ecotype | ER transformants | Null er control |
| Col-er105 | T8; T29; T19; T61 | T18 |
| Col-er106/er2/3401 | T165; T169; T279; T290 | T143 |
| Ld-er1 | T3-7K | NW20 |

TABLE 7
Carbon isotope composition (per mil) of 3 mature leaves, ground together harvested
Apr. 6, 2003 ie 32 days after sowing from still vegetative rosettes

| | T2 ER homozygous | | | | Null | | | background |
|---|---|---|---|---|---|---|---|---|
| | transformants | | | | transgenics (er) | | | er mutant |
| Line: | T46 | T29 | | | T18 | | | Col-er105 |
| | −31.4 | −31.2 | | | −32.2 | | | |
| Line: | T145 | T165 | T279 | T290 | T154 | T143 | T247 | Col er106/ |
| | | | | | | | | er2/3401 |
| | −30.4 | −31 | −30.5 | −30.8 | −31.5 | −32 | −31.7 | |
| Average: | | −30.6 | | | | −31.7 | | −31.7 |
| se: | | 0.15 | | | | 0.12 | | |
| line | T3-7K | | | | | | | Ld-er1 |
| | | | | | | | | NW20 |
| | −30.4 | | | | | | | −31.3 |

TABLE 8

| | | C isotopic composition | | | |
|---|---|---|---|---|---|
| | | (per mil) | | | |
| Erecta | Line | Run 14 | | Run 18 | |
| alleleLine | name | Average | St Err | Average | St Err |
| Col_er | 102K | | | −29.4 | .011 |
| mutants | 103K | | | −29.0 | .10 |
| | 105C | −31.4 | 0.12 | −30.3 | .09 |
| | 105KH | −30.2 | | −29.3 | 0.11 |
| | 105KS | −29.8 | 0.07 | −29.5 | .07 |
| | 3401 | −30 | 0.21 | −29.4 | .05 |
| | 106C | −30.2 | 0.04 | −29.4 | .12 |
| | 108K | −30.2 | 0.07 | −29.5 | .06 |
| | 111KH | −30.4 | 0 | −29.5 | .11 |
| | 111KS | −30.2 | 0.11 | −29.7 | .12 |
| | 114K | −30.2 | 0.11 | −29.5 | .04 |
| | 116K | −29.7 | 0.21 | −29.0 | .13 |
| | 117K | −29.7 | 0.18 | −29.3 | .08 |
| | 3140 | | | −32.2 | .06 |
| Col0_ER | 1093 | −29.6 | 0 | −29.0 | .10 |
| Ld_er1 | NW20 | −30.0 | 0.25 | −28.9 | .15 |
| | CS20 | −29.9 | 0.08 | | |
| Ld_ER Transgenic | 3177 | −29.4 | | −28.2 | .09 |
| Ld_er1 + wild type | | | | | |
| ER | 3-7K | −29.5 | 0.07 | −28.4 | .10 |

TABLE 9

| | C isotope composition (per mil) |
|---|---|
| Col_ER (line 933) | −28.1 |
| F1 (Col*Ld) | −28.5 |
| F1 (Ld*COL) | −29.3 |
| Ld-er1 (line NW20) | −29.9 |

1. A method of selecting a plant having enhanced transpiration efficiency, comprising detecting a genetic marker for transpiration efficiency which marker comprises a nucleotide sequence linked genetically to an ERECTA locus in the genome of the plant and selecting a plant that comprises or expresses the genetic marker.

2. The method according to claim 1 wherein the genetic marker comprises an ERECTA allele or erecta allele, or a protein-encoding portion thereof.

3. The method according to claim 2 wherein the genetic marker comprises a nucleotide sequence having at least about 55% overall sequence identity to at least about 20 nucleotides in length of any one of SEQ ID Nos: 1, 3, 5, 7, 9, 11 to 19 or 21 to 44 or a complementary sequence thereto.

4. The method according to claim 2 wherein the genetic marker comprises a nucleotide sequence selected from the group consisting of:
(a) a sequence having at least about 55% identity to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44;
(b) a sequence encoding an amino acid sequence having at least about 55% identity to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45; and
(c) a sequence complementary to (a) or (b).

5. The method according to claim 1 wherein the plant is selected from the group consisting of Arabidopsis thaliana, rice, sorghum, wheat and maize.

6. The method according to claim 1 comprising linking the transpiration efficiency phenotype of the plant to the expression of the marker in the plant.

7. The method according to claim 1 comprising linking a structural polymorphism in DNA to a transpiration efficiency phenotype in the plant.

8. The method according to claim 7 wherein the polymorphism is determined by a process comprising detecting a restriction fragment length polymorphism (RFLP), amplified fragment length polymorphism (AFLP), single strand chain polymorphism (SSCP) or microsatellite analysis.

9. The method according to claim 1 comprising hybridizing a probe or primer of at least about 20 nucleotides in length from any one of SEQ ID Nos: 1, 3, 5, 7, 9, 11 to 19 or 21 to 44 or a complementary sequence thereto to genomic DNA from the plant, and detecting the hybridization using a detection means.

10. The method according to claim 1 wherein the selected plant has enhanced transpiration efficiency compared to a near-isogenic plant that does not comprise or express the genetic marker.

11. A method of selecting a plant having enhanced transpiration efficiency, comprising:
(a) screening mutant or near-isogenic or recombinant inbred lines of plants to segregate alleles at an ERECTA locus;
(b) identifying a polymorphic marker linked to said ERECTA locus; and
(c) selecting a plant that comprises or expresses the marker.

12. A method of modulating the transpiration efficiency of a plant comprising introducing an isolated ERECTA gene or an allelic variant thereof or the protein-encoding region thereof to a plant and selecting a plant having a different transpiration efficiency compared to a near-isogenic plant that does not comprise the introduced ERECTA gene or allelic variant or protein-encoding region.

13. The method according to claim 12 wherein the ERECTA gene or allelic variant or protein-encoding region comprises a nucleotide sequence selected from the group consisting of:
(a) a sequence having at least about 55% identity to a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19; SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 SEQ ID NO: 33, SEQ ID NO: 34, SEQ ID NO: 35, SEQ ID NO: 36, SEQ ID NO: 37, SEQ ID NO: 38; SEQ ID NO: 39, SEQ ID NO: 40, SEQ ID NO: 41, SEQ ID NO: 42, SEQ ID NO: 43 and SEQ ID NO: 44; and
(b) a sequence encoding an amino acid sequence having at least about 55% identity to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 20 and SEQ ID NO: 45.

14. The method according to claim 12 wherein the plant is selected from the group consisting of Arabidopsis thaliana, rice, sorghum, wheat and maize.

15. The method according to claim 12 wherein the ERECTA gene or allelic variant or protein-encoding region is introduced to the plant by a process comprising introgression.

16. The method according to claim 12 wherein the ERECTA gene or allelic variant or protein-encoding region is introduced to the plant by a process comprising transforming plant material with a gene construct comprising the gene or allelic variant or protein-encoding region thereof.

17. The method according to claim 12 further comprising expressing the introduced gene or allelic variant or protein encoding region in the plant.

18. The method according to claim 12 wherein transpiration efficiency is enhanced in the plant.

19. The method of claim 18 wherein the transpiration efficiency is enhanced as a consequence of the ectopic expression of an ERECTA allele or the protein-encoding region thereof in the plant.

20. The method according to claim 12 wherein transpiration efficiency is reduced in the plant.

21-36. (canceled)


SEQUENCE LISTING (65 sequences)

SEQ ID NO: 1 - DNA, 3176 bp, Arabidopsis thaliana ERECTA allele
gtttcttcttcatggagacttgaaagcttttaaagtatatctaaaaacgcagtcgttttaagactgtgtgtgagaaatggctctgtttagagatattgttcttcttgggtttctcttctgcttgagcttagtagctactgtgacttcagaggagggagcaacgttgctggagattaagaagtcattcaaagatgtgaacaatgttctttatgactggacaacttcaccttcttcggattattgtgtctggagaggtgtgtcttgtgaaaatgtcaccttcaatgttgttgctcttaatttgtcagatttgaatcttgatggagaaatctcacctgctattggagatctcaagagtctcttgtcaattgatctgcgaggtaatcgcttgtctggacaaatccctgatgagattggtgactgttcttctttgcaaaacttagacttatccttcaatgaattaagtggtgacataccgttttcgatttcgaagttgaagcaacttgagcagctgattctgaagaataaccaattgataggaccgatcccttcaacactttcacagattccaaacctgaaaattctggacttggcacagaataaactcagtggtgagataccaagacttatttactggaatgaagttcttcagtatcttgggttgcgaggaaacaacttagtcggtaacatttctccagatttgtgtcaactgactggtctttggtattttgacgtaagaaacaacagtttgactggtagtatacctgagacgataggaaattgcactgccttccaggttttggacttgtcctacaatcagctaactggtgagatcccttttgacatcggcttcctgcaagttgcaacattatcattgcaaggcaatcaactctctgggaagattccatcagtgattggtctcatgcaagcccttgcagtcttagatctaagtggcaacttgttgagtggatctattcctccgattctcggaaatcttactttcaccgagaaattgtatttgcacagtaacaagctgactggttcaattccacctgagcttggaaacatgtcaaaactccattacctggaactcaatgataatcatctcacgggtcatataccaccagagcttgggaagcttactgacttgtttgatctgaatgtggccaacaatgatctggaaggacctatacctgatcatctgagctcttgcacaaatctaaacagcttaaatgttcatgggaacaagtttagtggcactataccccgagcatttcaaaagctagaaagtatgacttaccttaatctgtccagcaacaatatcaaaggtccaatcccggttgagctatctcgtatcggtaacttagatacattggatctttccaacaacaagataaatggaatcattccttcttcccttggtgatttggagcatcttctcaagatgaacttgagtagaaatcatataactggtgtagttccaggcgactttggaaatctaagaagcatcatggaaatagatctttcaaataatgatatctctggcccaattccagaagagcttaaccaattacagaacataattttgctgagactggaaaataataacctgactggtaatgttggttcattagccaactgtctcagtctcactgtattgaatgtatctcataacaacctcgtaggtgatatccctaagaacaataacttctcaagattttcaccagacagcttcattggcaatcctggtctttgcggtagttggctaaactcaccgtgtcatgattctcgtcgaactgtacgagtgtcaatctctagagcagctattcttggaatagctattgggggacttgtgatccttctcatggtcttaatagcagcttgccgaccgcataatcctcctccttttcttgatggatcacttgacaaaccagtaacttattcgacaccgaagctcgtcatccttcatatgaacatggcactccacgtttacgaggatatcatgagaatgacagagaatctaagtgagaagtatatcattgggcacggagcatcaagcactgtatacaaatgtgttttgaagaattgtaaaccggttgcgattaagcggctttactctcacaacccacagtcaatgaaacagtttgaaacagaactcgagatgctaagtagcatcaagcacagaaatcttgtgagcctacaagcttattccctctctcacttggggagtcttctgttctatgactatttggaaaatggtagcctctgggatcttcttcatggccctacgaagaaaaagactcttgattgggacacacggcttaagatagcatatggtgcagcacaaggtttagcttatctacaccatgactgtagtccaaggatcattcacagagacgtgaagtcgtccaacattctcttggacaaagacttagaggctcgtttgacagattttggaatagcgaaaagcttgtgtgtgtcaaagtcacatacttcaacttacgtgatgggcacgataggttacatagaccccgagtatgctcgcacttcacggctcactgagaaatccgatgtctacagttatggaatagtccttcttgagttgttaacccgaaggaaagccgttgatgacgaatccaatctccaccatctgataatgtcaaagacggggaacaatgaagtgatggaaatggcagatccagacatcacatcgacgtgtaaagatctcggtgtggtgaagaaagttttccaactggcactcctatgcaccaaaagacagccgaatgatcgacccacaatgcaccaggtgactcgtgttctcggcagttttatgctatcggaacaaccacctgctgcgactgacacgtcagcgacgctggctggttcgtgctacgtcgatgagtatgcaaatctcaagactcctcattctgtcaattgctcttccatgagtgcttctgatgctcaactgtttcttcggtttggacaagttatttctcagaacagtgagtagtttttcgttaggaggagaatctttaaaacggtatcttttcgttgcgttaagctgttagaaaaattaatgtctcatgtaaagtattatgcactgccttattattattagacaagtgtgtggtgtgaatatgtcttcagactggcacttagacttcctataagttcttgcc

SEQ ID NO: 2 - protein, 976 aa, Arabidopsis thaliana ERECTA allele
MALFRDIVLLGFLFCLSLVATVTSEEGATLLEIKKSFKDVNNVLYDWTTSPSSDYCVWRGVSCENVTFNVVALNLSDLNLDGEISPAIGDLKSLLSIDLRGNRLSGQIPDEIGDCSSLQNLDLSFNELSGDIPFSISKLKQLEQLILKNNQLIGPIPSTLSQIPNLKILDLAQNKLSGEIPRLIYWNEVLQYLGLRGNNLVGNISPDLCQLTGLWYFDVRNNSLTGSIPETIGNCTAFQVLDLSYNQLTGEIPFDIGFLQVATLSLQGNQLSGKIPSVIGLMQALAVLDLSGNLLSGSIPPILGNLTFTEKLYLHSNKLTGSIPPELGNMSKLHYLELNDNHLTGHIPPELGKLTDLFDLNVANNDLEGPIPDHLSSCTNLNSLNVHGNKFSGTIPRAFQKLESMTYLNLSSNNIKGPIPVELSRIGNLDTLDLSNNKINGIIPSSLGDLEHLLKMNLSRNHITGVVPGDFGNLRSIMEIDLSNNDISGPIPEELNQLQNIILLRLENNNLTGNVGSLANCLSLTVLNVSHNNLVGDIPKNNNFSRFSPDSFIGNPGLCGSWLNSPCHDSRRTVRVSISRAAILGIAIGGLVILLMVLIAACRPHNPPPFLDGSLDKPVTYSTPKLVILHMNMALHVYEDIMRMTENLSEKYIIGHGASSTVYKCVLKNCKPVAIKRLYSHNPQSMKQFETELEMLSSIKHRNLVSLQAYSLSHLGSLLFYDYLENGSLWDLLHGPTKKKTLDWDTRLKIAYGAAQGLAYLHHDCSPRIIHRDVKSSNILLDKDLEARLTDFGIAKSLCVSKSHTSTYVMGTIGYIDPEYARTSRLTEKSDVYSYGIVLLELLTRRKAVDDESNLHHLIMSKTGNNEVMEMADPDITSTCKDLGVVKKVFQLALLCTKRQPNDRPTMHQVTRVLGSFMLSEQPPAATDTSATLAGSCYVDEYANLKTPHSVNCSSMSASDAQLFLRFGQVISQNSE

SEQ ID NO: 3 - DNA, 3000 bp, rice ERECTA
atggcggcggcgagggcgccgtggctgtggtggtgggtggtggtggttgttggtgtggcggtggcggaggcggcctccggaggaggaggagggggagatggggaggggaaggcgctgatgggcgtgaaggccggtttcgggaacgcggccaacgcgctcgtcgactgggacggcggcgccgaccactgcgcgtggcgcggcgtcacctgcgacaacgcctccttcgccgtcctcgccctgaacttgtcaaatctaaacctaggaggtgagatctcgccggccatcggagagctcaagaatctacagttcgttgatctcaaggggaacaagctcactggccaaatcccagatgagattggggactgcatctccttaaaatatttggatttgtctggcaacttgctgtatggagacatccccttctccatctccaagctcaagcagcttgaggagctgattttgaagaacaaccagctcacgggacccatcccttccacattgtcccaaattccaaatctcaagacattggacctggcacagaaccagcttacaggcgatatcccaaggctcatatactggaatgaagttctgcaatacctaggtttgaggggtaactcactgactggaactttgtcacctgacatgtgccaactgactggcctgtggtactttgatgtaaggggaaacaatctcacagggaccattccagagagcatagggaactgcaccagctttgagattctggacatttcgtataaccaaatctctggagaaataccttacaacataggctttcttcaagtagccacactgtcacttcaaggaaatagactgactgggaaaattccagatgtgattggcctgatgcaagctcttgctgttctagacctgagtgagaacgagctggtagggcccattccttctatactgggcaatctatcctatactggaaaactatatttacatgggaacaaacttactggagtcataccgccggagcttgggaacatgagtaaacttagctacctacaactgaatgataatgaattggtgggcacaattccagcagagcttggcaaacttgaagagctttttgaactaaatcttgccaacaacaatcttcaaggtcctattcctgcaaacatcagttcttgcactgctctaaacaaattcaatgtttatggcaataagctaaatggttctattcctgctggtttccagaagttggagagtctgacttacttgaacctatcttcaaacaatttcaaaggcaatattccttctgagcttggtcacatcatcaacttggacacattggatctttcctacaatgaattctctggaccagttcctgctaccattggtgatctagagcaccttcttgaactgaatttgagtaagaaccatcttgatgggccagttcctgctgagtttggaaacttgagaagcgtccaagtaattgatatgtccaacaacaacttatctggtagtctgcccgaggaacttggacaacttcaaaaccttgatagcctgattcttaacaacaacaatttggttggggagatccctgctcaattggccaactgcttcagcttaaataaccttgcatttcaggaatttgtcatacaacaatttatctggacatgtcccgatggcaaagaacttctcgaaattcccaatggaaagcatcttctaatttctgattgcaaccagtacataaatcataaatgcagcttcttgggtaatccattactgcatgtttactgccaagattccagctgtggacactctcatggacaaagagttaatatttcaaagacagcaattgcttgcattatcttaggctttatcatattgctctgcgttctgctgttggctatatataaaacaaatcaaccacagccacttgtcaaaggatccgataagccagtgcaaggacctccaaagctagttgttctccagatggacatggctatccatacttacgaggacatcatgaggctgacagagaatttgagcgagaaatacatcattggctatggcgcctcaagcactgtctacaaatgtgaactcaagagcggcaaggccattgctgtcaagcggctttacagtcagtataaccatagcctccgagagtttgaaacagaactagagacaattggcagcatacggcacaggaatcttgttagcctccatggcttctcgctatctccacatggaaacttgctcttctatgattacatggaaaatggttccttgtgggatcttctccacggtccatcaaagaaagtgaagctcaactgggacacaagactgaggatcgcggtcggagctgcacaagggctggcctatctccaccatgactgcaaccctcgcataatccacagagatgtcaagtcctccaacatcctgctcgacgagaacttcgaagcgcacctctcagatttcggcatagccaaatgtgtcccctctgccaagtcccatgcctccacttatgtgctaggaaccatcggctacattgatccggagtatgccaggacttccaggctcaatgagaaatctgatgtgtacagcttcggcatcgtccttctggaattgctcacagggaagaaggccgtcgacaacgaatcgaacttgcatcaattgatactctccaaagctgatgacaacacagtcatggaggcagtggactcggaggtgtcagtgacgtgcacggacatgggactggtcaggaaggccttccagctcgcccttctgtgcaccaagaggcacccttcagaccggccgaccatgcacgaggttgcaagggtgctgctctccctgctgccggcctccgccatgacaacgcccaagacggtggactactcccggttgctggcgtcgacgacgacggcggccgacatgcgagggcacgacgtgaccgacatcggcgacaacagctcctccgacgagcagtggttcgtcaggttcggcgaggtcatatccaagcacacaatgtga

SEQ ID NO: 4 - protein, 999 aa, rice ERECTA
MAAARAPWLWWWVVVVVGVAVAEAASGGGGGGDGEGKALMGVKAGFGNAANALVDWDGGADHCAWRGVTCDNASFAVLALNLSNLNLGGEISPAIGELKNLQFVDLKGNKLTGQIPDEIGDCISLKYLDLSGNLLYGDIPFSISKLKQLEELILKNNQLTGPIPSTLSQIPNLKTLDLAQNQLTGDIPRLIYWNEVLQYLGLRGNSLTGTLSPDMCQLTGLWYFDVRGNNLTGTIPESIGNCTSFEILDISYNQISGEIPYNIGFLQVATLSLQGNRLTGKIPDVIGLMQALAVLDLSENELVGPIPSILGNLSYTGKLYLHGNKLTGVIPPELGNMSKLSYLQLNDNELVGTIPAELGKLEELFELNLANNNLQGPIPANISSCTALNKFNVYGNKLNGSIPAGFQKLESLTYLNLSSNNFKGNIPSELGHIINLDTLDLSYNEFSGPVPATIGDLEHLLELNLSKNHLDGPVPAEFGNLRSVQVIDMSNNNLSGSLPEELGQLQNLDSLILNNNNLVGEIPAQLANCFSLNNLAFQEFVIQQFIWTCPDGKELLEIPNGKHLLISDCNQYINHKCSFLGNPLLHVYCQDSSCGHSHGQRVNISKTAIACIILGFIILLCVLLLAIYKTNQPQPLVKGSDKPVQGPPKLVVLQMDMAIHTYEDIMRLTENLSEKYIIGYGASSTVYKCELKSGKAIAVKRLYSQYNHSLREFETELETIGSIRHRNLVSLHGFSLSPHGNLLFYDYMENGSLWDLLHGPSKKVKLNWDTRLRIAVGAAQGLAYLHHDCNPRIIHRDVKSSNILLDENFEAHLSDFGIAKCVPSAKSHASTYVLGTIGYIDPEYARTSRLNEKSDVYSFGIVLLELLTGKKAVDNESNLHQLILSKADDNTVMEAVDSEVSVTCTDMGLVRKAFQLALLCTKRHPSDRPTMHEVARVLLSLLPASAMTTPKTVDYSRLLASTTTAADMRGHDVTDIGDNSSSDEQWFVRFGEVISKHTM

SEQ ID NO: 5 - DNA, 2766 bp, sorghum ERECTA
atgacgacgacggccgcccgtgctctcgtcgccctcctcctcgtcgccgtcgccgtcgccgacgatggggcgacgctggtggagatcaagaagtccttccgcaacgtcggcaacgtactgtacgattgggccggcgacgactactgctcctggcgcggcgtcctgtgcgacaacgtcacattcgccgtcgctgcgctcaacctctctggcctcaaccttgagggcgagatctctccagccgtcggcagcctcaagagcctcgtctccatcgatctgaagtcaaatgggctatccgggcagatccctgatgagattggtgattgttcatcacttaggacgctggacttttctttcaacaacttggatggcgacataccattttctatatcaaagctgaagcacctggagaacttgatattgaagaacaaccagctgattggtgcgatcccatcaacattgtcacagctcccaaatttgaagattttggatttggcacaaaacaaactgactggggagataccaaggcttatctactggaatgaggttcttcaatatcttgatgtgaagaacaatagcttgaccggggtgataccagacaccattgggaactgtacaagttttcaagtcttggatttgtcttacaaccgctttactggaccaatcccattcaacattggtttcctacaagtggctacactatccttgcaagggaacaagttcaccggtccaattccttcagtaattggtcttatgcaggctctcgctgttctagatctgagttacaaccaattatctggtcctataccatcaatactaggcaacttgacatacactgagaagctgtacatccaaggcaataagttaactgggtcgataccaccagagttaggaaatatgtcaacacttcattacctagaactgaacgataatcaacttactgggtcaattccaccagagcttggaaggctaacaggcttgtttgacctgaaccttgcgaataaccacctggaaggaccaattcctgacaacctaagttcatgtgtgaatctcaatagcttcaatgcttatggcaacaagttaaatgggaccattcctcgttcgttgcggaaacttgaaagcatgacctatttaaatctgtcatcaaacttcataagtggctctattcctattgagttatcaaggatcaacaatttggacacgctggatttatcctgtaacatgatgactggtccaattccatcatcaattggcagcctagagcatctattgagacttaacttgagcaagaatggtctagttggattcatccccgcggagtttggtaatttgaggagtgtcatggagattgatttatcctataatcaccttggtggcctgattcctcaagaacttgaaatgctgcaaaacctgatgttgctaaatgtgtcgtacaataatttggctggtgttgtccctgctgacaacaacttcacacggttttcacctgacagctttttaggtaatcctggactctgtggatactggcttggttcgtcgtgtcgttccactggccaccacgagaaaccgcctatctcaaaggctgccataattggtgttgctgtgggtggacttgttatcctcttgatgatcttagtagctgtttgcaggccacatcgtccacctgcttttaaagatgtcactgtaagcaagccagtgagaaatgctccccccaagctggtgatccttcatatgaacatggcccttcatgtatacgatgacataatgaggatgactgagaacttgagtgagaaatacatcattggatacggggcgtcaagtacagtttataaatgtgtcctaaagaattgcaaaccggtggcaataaaaaagctgtatgcccactacccacagagccttaaggaatttgaaactgagcttgagactgttggtagcatcaagcaccggaatctagtcagccttcaagggtactcattatcacctgttgggaacctcctcttttatgattatatggaatgtggcagcttatgggatgttttacatgaaggttcatccaagaagaaaaaacttgactgggagactcgcctacggattgctcttggtgcagctcaaggccttgcttaccttcaccatgactgcagtccacggataattcatcgggatgtaaaatcaaagaatatactccttgacaaagattatgaggcccatcttacagactttggaattgctaagagcttatgtgtctcaaaaactcacacatcaacctatgtcatgggaactattggctacattgatcctgagtacgcccgcacttcccgtctcaacgaaaagtctgatgtctacaggctatggcattgttctgctggagctgctgactggcaagaagccagtggacaacgaatcctatcgaagacggcaagcaacgaggtcatggataccgtggaccctgacatcggggacacctgcaaggacctcggcgaggtgaagaagctgttccagctggcgctcctttgcaccaagcggcaaccctcggaccgaccgacgatgcacgaggtggtgcgcgtcctggactgcctggtgaacccggacccgccgccaaagccgtcggcgcaccagctgccgcagccgtcgccagccgtgccaagctacatcaacgagtacgtcagcctgcggggcaccggcgctctctcctgcgccaactcgaccagcacctcggacgccgagctgttcctcaagttcggcgaggccatctcgcagaacatggagtag

SEQ ID NO: 6 - protein, 921 aa, Sorghum ERECTA
MTTTAARALVALLLVAVAVADDGATLVEIKKSFRNVGNVLYDWAGDDYCSWRGVLCDNVTFAVAALNLSGLNLEGEISPAVGSLKSLVSIDLKSNGLSGQIPDEIGDCSSLRTLDFSFNNLDGDIPFSISKLKHLENLILKNNQLIGAIPSTLSQLPNLKILDLAQNKLTGEIPRLIYWNEVLQYLDVKNNSLTGVIPDTIGNCTSFQVLDLSYNRFTGPIPFNIGFLQVATLSLQGNKFTGPIPSVIGLMQALAVLDLSYNQLSGPIPSILGNLTYTEKLYIQGNKLTGSIPPELGNMSTLHYLELNDNQLTGSIPPELGRLTGLFDLNLANNHLEGPIPDNLSSCVNLNSFNAYGNKLNGTIPRSLRKLESMTYLNLSSNFISGSIPIELSRINNLDTLDLSCNMMTGPIPSSIGSLEHLLRLNLSKNGLVGFIPAEFGNLRSVMEIDLSYNHLGGLIPQELEMLQNLMLLNVSYNNLAGVVPADNNFTRFSPDSFLGNPGLCGYWLGSSCRSTGHHEKPPISKAAIIGVAVGGLVILLMILVAVCRPHRPPAFKDVTVSKPVRNAPPKLVILHMNMALHVYDDIMRMTENLSEKYIIGYGASSTVYKCVLKNCKPVAIKKLYAHYPQSLKEFETELETVGSIKHRNLVSLQGYSLSPVGNLLFYDYMECGSLWDVLHEGSSKKKKLDWETRLRIALGAAQGLAYLHHDCSPRIIHRDVKSKNILLDKDYEAHLTDFGIAKSLCVSKTHTSTYVMGTIGYIDPEYARTSRLNEKSDVYRLWHCSAGAADWQEASGQRILSKTASNEVMDTVDPDIGDTCKDLGEVKKLFQLALLCTKRQPSDRPTMHEVVRVLDCLVNPDPPPKPSAHQLPQPSPAVPSYINEYVSLRGTGALSCANSTSTSDAELFLKFGEAISQNME

SEQ ID NO: 7 - DNA, 2751 bp, Arabidopsis thaliana ERECTA homolog
atggcgataaaggcttcattcagcaacgtggcgaatatgcttcttgattgggacgatgttcataaccacgacttttgttcttggagaggtgtcttctgtgataacgttagcctcaatgttgtctctcttaatctgtcaaacctgaatcttggtggagagatatcatctgcccttggagatttgatgaatctgcaatcaatagacttgcaaggaaataaattgggtggtcaaattccagatgagattggaaactgtgtttctcttgcttatgtggatttctccaccaatttgttgtttggagacataccgttttcaatctctaaactcaaacagctgaccttaactcagattccaaaccttaagacccttgacctcgcaagaaaccagcttactggtgagataccaaggttactctactggaatgaagttttacagtatctcggtttacgtgggaatatgttaactgggacattgtctcctgatatgtgtcagctgacgggtctgtggtactttgatgtgagaggcaacaaccttactggaactatcccagagagcattggcaattgcacaagctttgagatcttggatgtatcttataatcagattaccggagttataccctacaatattggtttcctccaagtagctactctgtcacttcaaggaaacaagttgactggcagaattccggaagtgattggtctgatgcaggctcttgctgtattggatttgagtgacaatgaattaactgggcctattccaccaatacttgggaatctgtcattcactggaaaactgtatctccatggcaacaagctcactggacaaatcccacccgagctaggcaatatgtcacgactcagctatttgcaactaaatgataatgaactagtgggaaagatcccacctgagcttgggaagctggaacaattgttcgaactgaatcttgcgaacaacaatcttgtagggctgattccatctaacattagttcctgtgctgccttgaatcaattcaatgttcatgggaacttcttgagtggagctgtaccacttgaattccggaatcttggaagcttgacttatctaaatctttcctcaaacagtttcaagggcaaaatacctgctgagcttggccatatcatcaatcttgatacattggatctgtctggcaacaatttctcaggctcaattccattaacacttggtgatcttgagcatcttctcatcttaaacttgagcagaaatcatctgaatggcacattgcctgcagaattcgggaacctccgaagcattcagatcatcgatgtgtcatttaattttcttgccggtgttattccaactgaacttggccagttgcagaacataaactctctgatactgaacaacaacaagattcatgggaaaatccctgatcagctaactaactgcttcagtcttgccaatctgaacatctccttcaataatctttctggaataatcccacctatgaagaactttacacgtttttccccggccagcttctttggaaatccatttctctgcgggaactgggttggatcaatctgtggcccatctttacctaagtcacaagtattcaccagagttgccgtgatttgtatggttctcggtttcatcactctcatatgcatgatattcattgcggtttacaagtcaaagcagcagaaaccagtcttgaaaggctcttcaaaacaacctgaagggtcaacgaagctggtgattcttcacatggacatggctattcacacgtttgatgatatcatgagagttacagaaaacctcgatgagaaatacatcattggatacggtgcttctagcacagtttacaagtgcacctccaaaacttcccgacctattgccattaagcgaatctacaatcagtatcccagcaacttccgcgagtttgaaacagagctcgagaccattgggagcatcagacacagaaacatagtaagcttgcacggatacgccttatctccctttggcaacctcctcttctacgactacatggaaaatggctctctttgggatcttctccatgggcctgggaagaaggtgaagcttgactgggaaacaaggctgaagatagctgttggagctgcgcaaggacttgcatatcttcaccatgactgcacacctaggataatccatcgagacatcaagtcatcaaacatactccttgatgggaatttcgaagcgcgtttgtcagattttgggattgccaagagcataccagccaccaaaacttatgcttcaacctatgttcttggaaccattggatatattgacccagagtatgctcgaacttcgcgtctgaacgagaagtctgatatctacagtttcggtattgtccttcttgagcttctaaccggcaagaaggctgtggataacgaggccaacttgcatcaaatgattctatcaaaggcggatgataacacagtaatggaagctgttgatgcagaggtctcagtgacttgcatggactcaggacacatcaagaaaacatttcagctagctctcttgtgcaccaagcgaaatcctttggagagacccaccatgcaggaggtctctagggttctgctctcacttgtcccgtctccacctccaaagaagttaccgtcgcctgcaaaagtacaggaaggggaagaacggcgtgagagccactcttcagatacaacaaccccacagtggtttgttcagttccgtgaagatatctccaaaagtagcttataa

SEQ ID NO: 8 - protein, 932 aa, Arabidopsis thaliana ERECTA homolog
MAIKASFSNVANMLLDWDDVHNHDFCSWRGVFCDNVSLNVVSLNLSNLNLGGEISSALGDLMNLQSIDLQGNKLGGQIPDEIGNCVSLAYVDFSTNLLFGDIPFSISKLKQLEFLNLKNNQLTGPIPATLTQIPNLKTLDLARNQLTGEIPRLLYWNEVLQYLGLRGNMLTGTLSPDMCQLTGLWYFDVRGNNLTGTIPESIGNCTSFEILDVSYNQITGVIPYNIGFLQVATLSLQGNKLTGRIPEVIGLMQALAVLDLSDNELTGPIPPILGNLSFTGKLYLHGNKLTGQIPPELGNMSRLSYLQLNDNELVGKIPPELGKLEQLFELNLANNNLVGLIPSNISSCAALNQFNVHGNFLSGAVPLEFRNLGSLTYLNLSSNSFKGKIPAELGHIINLDTLDLSGNNFSGSIPLTLGDLEHLLILNLSRNHLNGTLPAEFGNLRSIQIIDVSFNFLAGVIPTELGQLQNINSLILNNNKIHGKIPDQLTNCFSLANLNISFNNLSGIIPPMKNFTRFSPASFFGNPFLCGNWVGSICGPSLPKSQVFTRVAVICMVLGFITLICMIFIAVYKSKQQKPVLKGSSKQPEGSTKLVILHMDMAIHTFDDIMRVTENLDEKYIIGYGASSTVYKCTSKTSRPIAIKRIYNQYPSNFREFETELETIGSIRHRNIVSLHGYALSPFGNLLFYDYMENGSLWDLLHGPGKKVKLDWETRLKIAVGAAQGLAYLHHDCTPRIIHRDIKSSNILLDGNFEARLSDFGIAKSIPATKTYASTYVLGTIGYIDPEYARTSRLNEKSDIYSFGIVLLELLTGKKAVDNEANLHQMILSKADDNTVMEAVDAEVSVTCMDSGHIKKTFQLALLCTKRNPLERPTMQEVSRVLLSLVPSPPPKKLPSPAKVQEGEERRESHSSDTTTPQWFVQFREDISKSSL

SEQ ID NO: 9 - DNA, 2901 bp, Arabidopsis thaliana ERECTA homolog
atgaaggagaagatgcagcgaatggttttatctttagcaatggtgggttttatggtttttggtgttgcttcggctatgaacaacgaagggaaagctctgatggcgataaaaggctctttcagcaacttagtgaatatgcttttggattgggacgatgttcacaacagtgacttgtgttcttggcgaggtgttttctgcgacaacgttagctactccgttgtctctctgaatttgtccagtctgaatcttggaggggagatatctccagctattggagacctacggaatttgcaatcaatagacttgcaaggtaataaactagcaggtcaaattccagatgagattggaaactgtgcttctcttgtttatctggatttgtccgagaatctgttatatggagacatacctttctcaatctctaaactcaagcagcttgaaactctgaatctgaagaacaatcagctcacaggtcctgtaccagcaaccttaacccagattccaaaccttaagagacttgatcttgctggcaatcatctaacgggtgagatatcgagattgctttactggaatgaagttttgcagtatcttggattacgagggaatatgttgactggaacgttatcttctgatatgtgtcagctaaccggtttgtggtactttgatgtgagaggaaataatctaactggaaccatcccggagagcatcggaaattgcacaagctttcaaatcctggacatatcttataatcagataacaggagagattccttacaatatcggcttcctccaagttgctactctgtcacttcaaggaaacagattgacgggtagaattccagaagttattggtctaatgcaggctcttgctgttttggatttgagtgacaatgagcttgttggtcctatcccaccgatacttggcaatctctcatttaccggaaagttgtatctccatggcaatatgctcactggtccaatcccctctgagcttgggaatatgtcacgtctcagctatttgcagctaaacgacaataaactagtgggaactattccacctgagcttggaaagctggagcaattgtttgaactgaatcttgccaacaaccgtttagtagggcccataccatccaacattagttcatgtgcagccttgaatcaattcaatgttcatgggaacctcttgagtggatctattccactggcgtttcgcaatctcgggagcttgacttatctgaatctttcgtcgaacaatttcaagggaaaaataccagttgagcttggacatataatcaatcttgacaaactagatctgtctggcaataacttctcagggtctataccattaacgcttggcgatcttgaacaccttctcatattaaatcttagcagaaaccatcttagtggacaattacctgcagagtttgggaaccttcgaagcattcagatgattgatgtatcattcaatctgctctccggagttattccaactgaacttggccaattgcagaatttaaactctttaatattgaacaacaacaagcttcatgggaaaattccagatcagcttacgaactgcttcactcttgtcaatctgaatgtctccttcaacaatctctccgggatagtcccaccaatgaaaaacttctcacgttttgctccagccagctttgttggaaatccatatctttgtggaaactgggttggatctatttgtggtcctttaccgaaatctcgagtattctccagaggtgctttgatctgcattgttcttggcgtcatcactctcctatgtatgattttccttgcagtttacaaatcaatgcagcagaagaagattctacaaggctcctcaaaacaagctgaagggttaaccaagctagtgattctccacatggacatggcaattcatacatttgatgatatcatgagagtgactgagaatcttaacgaaaagtttataattggatatggtgcttctagcacggtatacaaatgtgcattaaaaagttcccgacctattgccattaagcgactctacaatcagtatccgcataacttgcgggaatttgagacagaacttgagaccattgggagcattaggcacagaaacatagtcagcttgcatggatatgccttgtctcctactggcaaccttcttttctatgactacatggaaaatggatcactttgggaccttcttcatgggtcattgaagaaagtgaagcttgattgggagacaaggttgaagatagcggttggagctgcacaaggactagcctatcttcaccacgattgtactcctcgaatcattcaccgtgacatcaagtcatcgaacatacttcttgatgagaatttcgaagcacatttatctgatttcgggattgctaagagcataccagctagcaaaacccatgcctcgacttatgttttgggaacaattggttatatagacccagagtatgctcgtacttcacgaatcaatgagaaatccgatatatacagcttcggtattgttcttcttgagcttctcactgggaagaaagcagtggataacgaagctaacttgcatcaactgatattgtcaaaggctgatgataatactgtgatggaagcagttgatccagaggttactgtgacttgtatggacttgggacatatcaggaagacatttcagctggctctcttatgcacaaagcgaaaccctttagagagacccacaatgcttgaagtctctagggttctgctctctcttgtcccatctctgcaagtagcaaagaagctaccttctcttgatcactcaaccaaaaagctgcagcaagagaatgaagttaggaatcctgatgcagaagcatctcaatggtttgttcagttccgtgaagtcatctccaaaagtagcatataa

SEQ ID NO: 10 - protein, 966 aa, Arabidopsis thaliana ERECTA homolog
MKEKMQRMVLSLAMVGFMVFGVASAMNNEGKALMAIKGSFSNLVNMLLDWDDVHNSDLCSWRGVFCDNVSYSVVSLNLSSLNLGGEISPAIGDLRNLQSIDLQGNKLAGQIPDEIGNCASLVYLDLSENLLYGDIPFSISKLKQLETLNLKNNQLTGPVPATLTQIPNLKRLDLAGNHLTGEISRLLYWNEVLQYLGLRGNMLTGTLSSDMCQLTGLWYFDVRGNNLTGTIPESIGNCTSFQILDISYNQITGEIPYNIGFLQVATLSLQGNRLTGRIPEVIGLMQALAVLDLSDNELVGPIPPILGNLSFTGKLYLHGNMLTGPIPSELGNMSRLSYLQLNDNKLVGTIPPELGKLEQLFELNLANNRLVGPIPSNISSCAALNQFNVHGNLLSGSIPLAFRNLGSLTYLNLSSNNFKGKIPVELGHIINLDKLDLSGNNFSGSIPLTLGDLEHLLILNLSRNHLSGQLPAEFGNLRSIQMIDVSFNLLSGVIPTELGQLQNLNSLILNNNKLHGKIPDQLTNCFTLVNLNVSFNNLSGIVPPMKNFSRFAPASFVGNPYLCGNWVGSICGPLPKSRVFSRGALICIVLGVITLLCMIFLAVYKSMQQKKILQGSSKQAEGLTKLVILHMDMAIHTFDDIMRVTENLNEKFIIGYGASSTVYKCALKSSRPIAIKRLYNQYPHNLREFETELETIGSIRHRNIVSLHGYALSPTGNLLFYDYMENGSLWDLLHGSLKKVKLDWETRLKIAVGAAQGLAYLHHDCTPRIIHRDIKSSNILLDENFEAHLSDFGIAKSIPASKTHASTYVLGTIGYIDPEYARTSRINEKSDIYSFGIVLLELLTGKKAVDNEANLHQLILSKADDNTVMEAVDPEVTVTCMDLGHIRKTFQLALLCTKRNPLERPTMLEVSRVLLSLVPSLQVAKKLPSLDHSTKKLQQENEVRNPDAEASQWFVQFREVISKSSI

SEQ ID NO: 11 - DNA, 636 bp, partial wheat ERECTA
atgaattctgcaatacctaggcttgaggggtaactcactgactggaaccttgtcacctgacatgtgccaactcactggcctgtggtactttgatgtgaggggcaacaatctaacaggaacaattccacagagcatagggaactgcactagctttgagattctggacatttcatataacaaaatctctggagaaataccttacaacataggtttccttcaagtagctacactgtcacttcaaggaaatagactgactgggaaaattccagaagtgattggcctcatgcaagctcttgctgttcttgatctgagcgaaaacgaactagtaggggccattcctccgatactcggcaacctgtcctacactggcaaactatatttgcatggcaataaacttactggtgaagtacccccggaacttgggaacatgactaaacttagctacctgcaactgaatgacaatgaattagtgggcgcaattccagctgagcttgggaaacttgaagagctattcgaattaaatcttgccaacaacaatcttgagggtcctattcctacaaacatcagttcttgcactgcactaaacaaattcaatgtttacggcaatagattgaacggttctatccctgctggttt

SEQ ID NO: 12 - DNA, 466 bp, partial wheat ERECTA
ttcaatgtttatggcaatagattgaacggttctatccctgctggtttccagaatttggagagtttgactaacttgaatttatcctcaaacaattttaaaggccatatcccatctgaacttggtcatatcatcaatttggacacactggatctttcctacaatgaactctctggaccagttcctgctactattggtgatcttgagcatcttcttcaactaaatttgagcaaaaaccatcttagcgggtcagtgcctgctgagttcggaaacttgagaagcatccaagtaattgatttatccaacaacgccatgtctggttatctccctgaagaactaggccaacttcagaaccttgatagtttgattcttaacaacaatattttggtcggagagatccctgctcagttggctaactgcttcagcttaaacatcttgaacttgtcacataacaacttttctggacatg

SEQ ID NO: 13 - DNA, 372 bp, partial wheat ERECTA
  misc_feature (62)..(62): not determined
ttgtcagccttctggcttctcactctctcccaatggaaacctgctcttctacgattacatgngaaaacggttccttgtgggatcttctccatggtccatcaaagaaagtgaagcttgactgggacacccgactgaggatcgcggtcggcgcggcacaagggctggcctatctgcaccatgactgcaatctgcggatagtccacagggacgtcaagtcctccaacatcctgctcgacgagcactttgaagcgcatctctcggacttcggcatcgccaaatgcgtcccggcagccaagacccatgcgtccacatatgtgctaggaaccatcggctacatcgatccagagtacgcccggacgtcgaggttgaacg

SEQ ID NO: 14 - DNA, 357 bp, partial wheat ERECTA
ttgactaacttgaatttatcctcaaacaattttaaaggccatatcccatctgaacttggtcatatcatcaatttggacacactggatctttcctacaatgaactctctggaccagttcctgctactattggtgatcttgagcatcttcttcaactaaatttgagcaaaaaccatcttagcgggtcagtgcctgctgagttcggaaacttgagaagcatccaagtaattgatttatccaacaacgccatgtctggttatctccctgaagaactaggccaacttcagaaccttgatagtttgattcttaacaacaatattttggtcggggagatccctgctcagttggctaactgcttc

SEQ ID NO: 15 - DNA, 314 bp, partial wheat ERECTA
cacactggatctttcctacaatgaattctctggaccagaccctgctactattggtgatcttgagcatgttcttcagattaaatttgagcaaaaaccatcttactgggccaatgcctgctgagttctgaaacttgagaagcatccaagtaattgatttatccaacaacgccatgtctggttatctccctgaagaactacgccaacttcagaatcttgatagtttgatgcttaacaacaatactttggttggggagatccctgctcatctggctaactgcttcaacttaaacatcttgaacttgccatataacaac

SEQ ID NO: 16 - DNA, 549 bp, partial wheat ERECTA
catcatcggctacggcgcttcaagtaccgtgtataaatgtgtgctcaagagtggcaaggccattgctgtgaagcggctctacagccaatacaaccatggcgcccgtgagtttgagacagagctggagacagtcggtagcatccggcacaggaatcttgtcagccttcatggcttctcactctctccaaatggaaacctgctcttctacgattacatggaaaatggttccttgtgggatcttctccacggtccatcaaagaaggtgaaacttgactgggacacccgactgagaatcgccgtcggtgcggcacaagggctggcatatcttcaccatgactgcaaccctcggatagtccacagggacgtcaagtcctccaacatcctgctcgacgagcactttgaagcgcatctctcggacttcggcatcgccaaatgcgtcccagctgccaagacccacgcgtccacctatgtgctaggaaccatcggctacatcgatccagagtacgcccggacgtcccagctgaacgagaaatctgatgtgtacagctt

SEQ ID NO: 17 - DNA, 615 bp, partial wheat ERECTA
ccacgcgtcgatcatcaattgggacacacgggatctttcctacaatgaattctccgggccagttcctgctactattggtgatctggagcatcttcttcaactaaatttgagcaaaaaccatcttagtgggtctgtgcctgctgagttcggaaacttgagaagcatccaagtaattgatttatccaacaacgccatttctggttatctccctgaagaactaggccaacttcagaaccttgatagtttgattcttaacaacaatactttggttggggagatccctgctcagttggctaactgcttcagcttaaacatcttgaacttgtcatataacaacttttctggacatgtcccattcgctaagaacttctcaaagttccccggggaaagcttcttgggaaatccgatgctgagcgttcactgcaaagactccagctgtggcaactctcatggatcaaaagtgaatactcggacagcgattgcttgcatcatctcgggcttcgtcatattgctctgtgttctgctattgggcaatatataaaacaaagcgaccacagccacctatcaaagcatctgataaaccagggcaaggacctccaaagatagtactcctcc

SEQ ID NO: 18 - DNA, 719 bp, partial wheat ERECTA
cgttcactgcaaagactccagctgtggcaactctcatggatcaaaagtgaatattcggacggcgattgcttgcatcatctcgggcttcgtcatactgctatgtgttctgctattggcaatatataaaacaaagcgaccacagccacctatcaaagcatctgataaaccagtgcaaggacctccaaagatagtactcctccaaatggacatggctatccatacctatgatgatattatgaggctgacagagaatttgagcgagaaatacatcatcggctacggcgcttcaagtaccgtgtataaatgtgtgctcaagagtggcaaggccattgctgtgaagcggctctacagccaatacaaccatggcgcccgtgagtttgagacagagctggagacagtcggtagcatccggcacaggaatcttgtcagccttcatggcttctcactctctccaaatggaaacctgctcttctacgattacatggaaaatggttccttgtgggatcttctccacggtccatcaaagaaggtgaaacttgactgggacacccgactgagaatcgccgtcggtgcggcacaagggctggcatatcttcaccatgactgcaaccctcggatagtccacagggacgtcaagtcctccaacatcctgctcgacgagcactttgaagcgcatctctcggacttcggcatcgcccaatgcgtccccagctgccaag

SEQ ID NO: 19 - DNA, 1346 bp, wheat ERECTA
ttcaatgtttatggcaatagattgaacggttctatccctgctggtttccagaatttggagagtttgactaacttgaatttatcctcaaacaattttaaaggccatatcccatctgaacttggtcatatcatcaatttggacacactggatctttcctacaatgaactctctggaccagttcctgctactattggtgatcttgagcatcttcttcaactaaatttgagcaaaaaccatcttagcgggtcagtgcctgctgagttcggaaacttgagaagcatccaagtaattgatttatccaacaacgccatgtctggttatctccctgaagaactaggccaacttcagaaccttgatagtttgattcttaacaacaatattttggtcggggagatccctgctcagttggctaactgcttcagcttaaacatcttgaacttgtcacataacaacttttctggacatgtcccattcgctaagaacttctcaaagttccccggggaaagcttcttgggaaatccgatgctgagcgttcactgcaaagactccagctgtggcaactctcatggatcaaaagtgaatactcggacagcgattgcttgcatcatctcgggcttcgtcatactgctatgtgttctgctattggcaatatataaaacaaagcgaccacagccacctatcaaagcatctgataaaccagggcaaggacctccaaagatagtactcctccaaatggacatggctatccatacctatgatgatattatgaggctgacagagaatttgagcgagaaatacatcatcggctacggcgcttcaagtaccgtgtataaatgtgtgctcaagagtggcaaggccattgctgtgaagcggctctacagccaatacaaccatggcgcccgtgagtttgagacagagctggagacagtcggtagcatccggcacaggaatcttgtcagccttcatggcttctcactctctccaaatggaaacctgctcttctacgattacatggaaaatggttccttgtgggatcttctccacggtccatcaaagaaggtgaaacttgactgggacacccgactgagaatcgccgtcggtgcggcacaagggctggcatatcttcaccatgactgcaaccctcggatagtccacagggacgtcaagtcctccaacatcctgctcgacgagcactttgaagcgcatctctcggacttcggcatcgccaaatgcgtcccagctgccaagacccacgcgtccacatatgtgctaggaaccatcggctacatcgatccagagtacgcccggacgtcccagctgaacgagaaatctgatgtgtacagctt

SEQ ID NO: 20 - protein, 448 aa, wheat ERECTA
FNVYGNRLNGSIPAGFQNLESLTNLNLSSNNFKGHIPSELGHIINLDTLDLSYNELSGPVPATIGDLEHLLQLNLSKNHLSGSVPAEFGNLRSIQVIDLSNNAMSGYLPEELGQLQNLDSLILNNNILVGEIPAQLANCFSLNILNLSHNNFSGHVPFAKNFSKFPGESFLGNPMLSVHCKDSSCGNSHGSKVNTRTAIACIISGFVILLCVLLLAIYKTKRPQPPIKASDKPGQGPPKIVLLQMDMAIHTYDDIMRLTENLSEKYIIGYGASSTVYKCVLKSGKAIAVKRLYSQYNHGAREFETELETVGSIRHRNLVSLHGFSLSPNGNLLFYDYMENGSLWDLLHGPSKKVKLDWDTRLRIAVGAAQGLAYLHHDCNPRIVHRDVKSSNILLDEHFEAHLSDFGIAKCVPAAKTHASTYVLGTIGYIDPEYARTSQLNEKSDVYS

SEQ ID NO: 21 - DNA, 1273 bp, partial maize ERECTA
cctggactctgtggatattggcttggttcttcatgtcgttccactggccaccgagacaaaccgccaatctcaaaggctgccataattggtgttgctgtgggtggacttgttatcctcctgatgatcttagtagctgtatgcaggccacaccatccacctgcttttaaagatgccactgtaagcaagccagtgagcaatggtccacccaagctggtgatccttcatatgaacatggctcttcatgtctttgatgatataatgaggatgactgagaacttgagtgagaaatacatcattggatacggggcatcaagtacagtttataaatgtgttctaaagaattgcaaaccagtggcaataaaaaagctgtatgcccactaccctgcagagccttaaggaatttgaaactgagctcgagactgttggtagcatcaaacaccggaatctagtcagccttgccaagggtactcgttgtcacctgttgggaacctcctcttttatgattatatggagagtggcagcttatgggatgttttacatgaaggctcatccaagaagaacaaacttgactgggtgactcgcctacggatcgctcttggtgcagctcaaggcctcgcttaccttcaccatgactgcagcccacgaataattcaccgggacgtaaaatcaaagaatatactcctcgacaaagattatgaggcccatcttacagacttcggcatcgctaagagcttatgtgtctcgaagactcacacgtcaacctacgtcatgggcactattggttacattgatcccgagtacgcccgcacctcccgcctcaacgagaagtctgatgtctacagctacggcatcgttctgctggagctgctgaccggcaagaagccagtggacaacgagtgcaatctccatcacttgatcctatcgaagacggcgagcaacgaggtcatggagacggtggaccccgacgtgggagacacctgcaaggacctgggcgaggtgaagaagctgttccagctggcgctcctctgcaccaagcggcagccctcggaccggccgacgatgcacgaggtggtgcgcgtccttgactgcctggtgaacccggagccgccgccgcagccgcagcagcagcagcagaagggcgcacgcgcaccaccagctgccgccgcagccgtcgccgccggcctacgtcgacgagtacgtcagcctgcggggcactggcgccctctcctgcgccaactcgtccagcacctcggacgccgagctgttcctcaag

SEQ ID NO: 22 - DNA, 100 bp, partial maize ERECTA
cacaaaatgtcagtcaaactactccccctgcaatcggcctcactcaaggcgcctcaccgaacgtctacgtcttcccctacaccatgttctgcgagatggc

SEQ ID NO: 23 - DNA, 599 bp, partial maize ERECTA
  misc_feature (529)..(529): not determined
tttttttttttttttttttttttttgaggaggaagctccgctgctcttgcgttgcgtcacatgactttttacagctaacaacaccctagctactgagtcccatgttaatctcctgcgctgcgtcccacaaaatgtcagtcaaactactccctgcaatcggcctcactcaaggcgcctcaccgaacgtctacgtcttcccctacaccatgttctgcgagatggcctcgccgaacttgaggaacagctcggcgtccgaggtgctggacgagttggcgcaggagagggcgccggtgccccgcaggctgacgtactcgtcgacgtaggccggcggcgacggctgcggcggcagctggtggtgcgcgtgcgccttctgctgctgctgctgcggctgcggcggcggctccgggttcaccaggcagtcaaggacgcgcaccacctcgtgcatcgtcggccggtccgagggctgccgcttggtgcagaggagcgccagctggaacagcttcttcacctcgcccaggtccttgcaggngtctcccacgtcggggtccaccgtctccatgacctcgttgctcgccgtcttcgataggatcaaggatgga

SEQ ID NO: 24 - DNA, 436 bp, partial maize ERECTA
ttttttttttttttttttgaagaagctccgctgctctcgcgttgcgtcacatgactttttacagataacaccaccctagctactgagtcccatgttaatctcctgcgctgcgttccacaaaatgtcagccaaactactccctgcaatcggcctcactcaaggcgcctcaccgaacgtctacgtcttcccctacaccatgttctgcgagatggcctcgccgaacttgaggaacagctcggcgtccgaggtgctggacgagttggcgcaggagagggcgccggtgccccgcaggctgacgtactcgtcgacgtaggccggcggggacggctgcggcggcagctggtggtgcgcgtgcgccttctgctgctgctgctgcggctgcggcgggggctccgggttcaccaggcagtcaaggacgcgcaccacctcgggcatc

SEQ ID NO: 25 - DNA, 509 bp, partial maize ERECTA
ccaaagaaaaatggagaggggggataaagaagatgaggaagaagctccgctgctcttgcgttgcgtcacatgactttttacagctaacaacaccctagctactgagtcccatgttaatctcctgcgctgcgtcccacaaaatgtcagtcaaactactccctgcaatcggcctcactcaaggcgcctcaccgaacgtctacgtcttcccctacaccatgttctgcgagatggcctcgccgaacttgaggaacagctcggcgtccgaggtgctggacgagttggcgcaggagagggcgccggtgccccgcaggctgacgtactcgtcgacgtaggccggcggcgacggctgcggcggcagctggtggtgcgcgtgcgccttctgctgctgctgctgcggctgcggcggcggctccgggttcaccaggcagtcaaggacgcgcaccacctcgtgcatcgtcggccggtccgagggctgccgcttggtgcagaggagcgccagctggaacagc

SEQ ID NO: 26 - DNA, 318 bp, partial maize ERECTA
gatggatcaatacagcctcctagtaagttagaccaccaaagaaaaatggggaggggggataaagaagaggaagaagctccgctgctcttgcgtcacatgactttttttacagctaacaacaccctagctactgagtcccatgttaatctcctgcgctgcgtcccacaaaatgtcagtcaaactactccccctgcaatcggcctcactcaaggcgcctcaccgaacgtctacgtcttcccctacaccatgttctgcgagatggcctcgccgaacttgaggaacagctcggcgtccgaggtgctggacgagttggcgcag

SEQ ID NO: 27 - DNA, 103 bp, partial maize ERECTA
agcaagccagtgagcaatggtccacccaagctggtgatccttcatatgaacatggctcttcatgtctttgatgatataatgaggatgactgagaacttgagtg

SEQ ID NO: 28 - DNA, 458 bp, partial maize ERECTA
ataattcaccgggacgtaaaatcaaagaatatactcctcgacaaagattatgaggcgcatcttacagacttcggcatcgctaagagcttatgtgtctcgaagactcacacgtcaacctacgtcatgggcactattggtacacttgatcctgagtacgcccgcacctcccgcctcaacgagaagtctgatgtctacagctacggcatcgttctgctggagctgctgaccggcaagaagccagtggacaacgagtgcaatctccatcacttgatcctatcgaagacggcgagccaacgaggtcatggagacggtggaccccgacgtgggagacacctgcaaggacctgggcgaggtgaagaagctgttccagctggcgctcctctgcaccaagcggcagccctcggaccggccgacgatgcacgaggtggtgcgcgtccttgactgcctggtgaacccgg

SEQ ID NO: 29 - DNA, 593 bp, partial maize ERECTA
ttttttttttttttttttttttttttgaggaagaagctccgctgctcttgcgttgcgtcacatgactttttacagctaacaacaccctagctactgagtcccatgttaatctcctgcgctgcgtcccacaaaatgtcagtcaaactactccctgcaatcggcctcatttttttgttgtcctcaccgaacgtctacgtcttcccctacaccatgttctgcgagatggcctcgccgaacttgaggaacagctcggcgtccgaggtgctggacgagttggcgcaggaaagggcgccggtgccccgcaggctgacgtactcgtcgacgtaggccggcggcgacggctgcggcggcagctggtggtgcgcgtgcgccttctgctgctgctgctgcggctgcggcggcggctccgggttcaccaggcagtcaaggacgcgcaccacctcgtgcatcgtcggccggtccgagggctgccgcttggtgcagaggagcgccagctggaacagcttcttcacctcgcccaggtccttgcaggtgtctcccacgtcggggtccaccggctccatgacctcgttgctcgccgtcttcgataggat

SEQ ID NO: 30 - DNA, 206 bp, partial maize ERECTA
tcacaaaagatcatcaagcagaggaacgggagagatgatgatggatcaatacagcctcctagtaagttagaccacaaagaaaaatggggaggggggataaagaagaggaagaagctccgctgctcttgcgtcacatgactttttttacagctaacaacaccctagctactgagtcccatgttaatctcctgcgctgcgtcccacaa

SEQ ID NO: 31 - DNA, 534 bp, partial maize ERECTA
caagcagaggaacgggagagatgatgatggatcaatacagcctcctagtaagttagaccacaaagaaaaatggggaggggggataaagaagaggaagaagctccgctgctcttgcgtcacatgactttttttacagctaacaacaccctagctactgagtcccatgttaatctcctgcgctgcgtcccacaaaatgtcagtcaaactactccccctgcaatcggcctcactcaaggcgcctcaccgaacgtctacgtcttcccctacaccatgttctgcgagatggcctcgccgaacttgaggaacagctcggcgtccgaggtgctggacgagttggcgcaggagagggcgccggtgccccgcaggctgacgtactcgtcgacgtaggccggcggcgacggctgcggcggcagctggtggtgcgcgtgcggcttctgctgctgctgctgcggctgcggcggcggctccgggttcaccaggcagtcaaggacgcgcaccacctcgtgcatcgtcggccggtccgagggctgccgc

SEQ ID NO: 32 - DNA, 527 bp, partial maize ERECTA
gaaagtcacaagatcataaggaagaggaacgggagagatgatgatggatcaatacagcctcctagtaagttagaccaccaaagaaaaatggagaggggggataaagaagatgaggaagaagctccgctgctcttgcgttgcgtcacatgactttttacagctaacaacaccctagctactgagtcccatgttaatctcctgcgctgcgtcccacaaaatgtcagtcaaactactccctgcaatcggcctcactcaaggcgcctcaccgaacgtctacgtcttcccctacaccatgttctgcgagatggcctcgccgaacttgaggaacagctcggcgtccgaggtgctggacgagttggcgcaggaaagggcgccggtgccccgcaggctgacgtactcgtcgacgtaggccggcggcgacggctgcggcggcagctggtggtgcgcgtgcgccttctgctgctgctgctgcggctgcggcggcggctccgggttcaccaggcagtcaaggacgcgcaccacctcgt

SEQ ID NO: 33 - DNA, 412 bp, partial maize ERECTA
cttgcgttgcgtcacatgactttttacagctaacaacaccctagctactgagtcccatgttaatctcctgcgctgcgtcccacaaaatgtcagtcaaactactccctgcaatcggcctcactcagggggcctcaccgaacgtctacgtcttcccctacaccaggttctgcgagatggcctcgccgaacttgaggaacagctcggcgtccgaggggctggacgagttggcgcaggaaagggcgccggggccccgcaggctgacgtactcgtcgacgtaggccggcggcgacggctgcggcggcagctgggggtgcgcgtgcgccttctgctgctgctgctgcggttgcggcggcggctccgggttcaccaggcagtcaaggacgcgcaccacctcgggcatcgtcggccggtc

SEQ ID NO: 34 - DNA, 533 bp, partial maize ERECTA
tcgagtttttttttttttttttttgatgatggatcaatacagcctcctagtaagttagaccaccaaagaaaaatggagaggggggataaagaagatgaggaagaagctccgctgctcttgcgttgcgtcacatgactttttacagctaacaacaccctagctactgagtcccatgttaatctcctgcgctgcgtcccacaaaatgtcagtcaaactactccctgcaatcggcctcactcaaggcgcctcaccgaacgtctacgtcttcccctacaccatgttctgcgagatggcctcgccgaacttgaggaacagctcggcgtccgaggtgctggacgagttggcgcaggagagggcgccggtgccccgcaggctgacgtactcgtcgacgtaggccggcggcgacggctgcggcggcagctggtggtgcgcgtgcgccttctgctgctgctgctgcggctgcggcggcggctccgggttcaccaggcagtcaaggacgcgcaccacctcgtgcatcgtcggccggtccgagg

SEQ ID NO: 35 - DNA, 191 bp, partial maize ERECTA
agcctcctagtaagttagaccaccaaagaaaaatggagaggggggataaagaagatgaggaagaagctccgctgctcttgcgttgcgtcacatgactttttacagctaaacaacaccctagctactgagtcccatggtaatctcctgcgctgcgtcccacaaaatgtcagtcaaactactccctgcaatcg

SEQ ID NO: 36 - DNA, 683 bp, partial maize ERECTA
gacgttgggaacctcctcttttatgctttatggagagtggcagcttatgggatgttttacatgaaggctcatccaagaagaacaaacttgactgggtgactcgcctacggatcgctcttggtgcagctcaaggcctcgcttaccttcaccatgactgcagcccacgaataattcaccgggacgtaaaatcaaagaatatactcctcgacaaagattatgaggcgcatcttacagacttcggcatcgctaagagcttatgtgtctcgaagactcacacgtcaacctacgtcatgggcactattggttacattgatcctgagtacgcccgcacctcccgcctcaacgagaagtctgatgtctacagctacggcatcgttctgctggagctgctgaccggcaagaagccagtggacaacgagtgcaatctccatcacttgatcctatcgaagacggcgagcaacgaggtcatggagacggtggaccccgacgtgggagacacctgcaaggacctgggcgaggtgaagaagctgttccagctggcgctcctctgcaccaagcggcagccctcggaccggccgacgatgcacgaggtggtgcgcgtccttgactgcctggtgaacccggagccgccgccgcagccgcagcagcagcagcagaaggcgcacgcgcaccaccagctgccg

SEQ ID NO: 37 - DNA, 610 bp, partial maize ERECTA
cttcggcatcgctaagagcttatgtgtctcgaagactcacacgtcaacctacgtcatgggcactattggttacattgatcctgagtacgcccgcacctcccgcctcaacgagaagtctgatgtctacagctacggcatcgttctgctggagctgctgaccggcaagaagccagtggacaacgagtgcaatctccatcacttgatcctatcgaagacggcgagcaacgaggtcatggagacggtggaccccgacgtgggagacacctgcaaggacctgggcgaggtgaagaagctgttccagctggcgctcctctgcaccaagcggcagccctcggaccggccgacgatgcacgaggtggtgcgcgtccttgactgcctggtgaacccggagccgccgccgcagccgcagcagcagcagcagaaggcgcacgcgcaccaccagctgccgccgcagccgtcgccgccggcctacgtcgacgagtacgtcagcctgcggggcaccggcgccctctcctgcgccaactcgtccagcacctcggacgccgagctgttcctcaagttcggcgaggccatctcgcagaacatggtgtaggggaagacgtagacgttc

SEQ ID NO: 38 - DNA, 208 bp, partial maize ERECTA
  misc_feature (138)..(138): not determined
gcaagccagtgagcaatggtccacccaagctgggatccttcatatgaacatggctcttcatgtctttgatgatataatgaggatgactgagaacttgagtgagaaatacatcattggatacggggcatcaagtactgnttataaatgtgttctaaagaattgcaaaccagtggcaataaaaaagctgtatgcccactaccctcagagc

SEQ ID NO: 39 - DNA, 634 bp, partial maize ERECTA
gaccgggacgtaaaatcaaagaatatactcctcgacaaagattatgaggcgcatcttacagacttcggcatcgctaagagcttatgtgtctcgaagactcacacgtcaacctacgtcatgggcactattggttacattgatcctgagtacgcccgcacctcccgcctcaacgagaagtctgatgtctacagctacggcatcgttctgctggagctgctgaccggcaagaagccagtggacaacgagtgcaatctccatcacttgatcctatcgaagacggcgagcaacgaggtcatggagacggtggaccccgacgtgggagacacctgcaaggacctgggcgaggtgaagaagctgttccagctggcgctcctctgcaccaagcggcagccctcggaccggccgacgatgcacgaggtggtgcgcgtccttgactgcctggtgaacccggagccgccgccgcagccgcagcagcagcagcagaaggcgcacgcgcaccaccagctgccgccgcagccgtcgccgccggcctacgtcgacgagtacgtcagcctgcggggcaccggcgccctctcctgcgccaactcgtccagcacctcggacgccgagctgttcctcaagttcggcgaggcca

SEQ ID NO: 40 - DNA, 558 bp, partial maize ERECTA
acttgatgccccgtatccaatgatgtatttctcactcaagttctcagtcatcctcattatatcatcaaagacatgaagagccatgttcatatgaaggatcaccagcttgggtggaccattgctcactggcttgcttacagtggcatctttaaaagcaggtggatggtgtggcctgcatacagctactaagatcatcaggaggataacaagtccacccacagcaacaccaattatggcagcctttgagattggcggtttgtctcggtggccagtggaacgacatgaagaaccaagccaatatccacagagtccaggattacctaaaaagctgtcatgtgaaaaccgtgtgaagttgttgtcagtagggacagcaccagccaaattattgtatgacacatttaagatattgaggctgaagcagttcatcagagaagagacatcgccagttatattgttgttttccagttttagcaacatcaggttttgcagcattccaagttcttgaggaatcagaccaccaagatgattataggataaatcaatctccatgacacttct

SEQ ID NO: 41 - DNA, 429 bp, partial maize ERECTA
tacttgatgccccgtatccaatgatgtatttctcactcaagttctcagtcatcctcattatatcatcaaagacatgaagagccatgttcatatgaaggatcaccagcttgggtggaccattgctcactggcttgcttacagtggcatctttaaaagcaggtggatggtgtggcctgcatacagctactaagatcatcaggaggataacaagtccacccacagcaacaccaattatggcagcctttgagattggcggtttgtctcggtggccagtggaacgacatgaagaaccaagccaatatccacagagtccaggattacctaaaaagctgtcatgtgaaaaccgtgtgaagttgttgtcagtagggacagcaccagccaaattattgtatgacacatttaagatattgaggctgaagcagttcatca

SEQ ID NO: 42 - DNA, 556 bp, partial maize ERECTA
acatgcaagtcaacaggttaactggatcgataccaccagagctaggaaatatgtcaacacttcattacctagaactgaatgataatcaacttactgggtcaattccaccagagcttggaaggctaacaggcttgtttgacctgaaccttgcgaataaccaccttgaaggaccaattcctgacaacctaagttcatgtgtgaatctcaatagcttcaatgcttatggcaacaagttaaatggaaccattcctcgttcgctgcggaaacttgaaagcatgacctatttaaatctttcatcaaatttcataagtggctctattcctattgagctatcaaggatcaacaatttggacacgttggacttatcctgtaacatgatgacgggtccaattccatcatccattggcaacctagagcatctattgaggcttaacttgagcaagaatgatctagttggattcatccctgcggagtttggtaatttgagaagtgtcatggagattgatttatcctataatcatcttggtggtctgattcctcaagaacttggaatgct

SEQ ID NO: 43 - DNA, 683 bp, partial maize ERECTA; identical to SEQ ID NO: 36

SEQ ID NO: 44 - DNA, 2315 bp, maize ERECTA
acatgcaagtcaacaggttaactggatcgataccaccagagctaggaaatatgtcaacacttcattacctagaactgaatgataatcaacttactgggtcaattccaccagagcttggaaggctaacaggcttgtttgacctgaaccttgcgaataaccaccttgaaggaccaattcctgacaacctaagttcatgtgtgaatctcaatagcttcaatgcttatggcaacaagttaaatggaaccattcctcgttcgctgcggaaacttgaaagcatgacctatttaaatctttcatcaaatttcataagtggctctattcctattgagctatcaaggatcaacaatttggacacgttggacttatcctgtaacatgatgacgggtccaattccatcatccattggcaacctagagcatctattgaggcttaacttgagcaagaatgatctagttggattcatccctgcggagtttggtaatttgagaagtgtcatggagattgatttatcctataatcatcttggtggtctgattcctcaagaacttggaatgctgcaaaacctgatgttgctaaaactggaaaacaacaatataactggcgatgtctcttctctgatgaactgcttcagcctcaatatcttaaatgtgtcatacaataatttggctggtgctgtccctactgacaacaacttcacacggttttcacatgacagctttttaggtaatcctggactctgtggatattggcttggttcttcatgtcgttccactggccaccgagacaaaccgccaatctcaaaggctgccataattggtgttgctgtgggtggacttgttatcctcctgatgatcttagtagctgtatgcaggccacaccatccacctgcttttaaagatgccactgtaagcaagccagtgagcaatggtccacccaagctggtgatccttcatatgaacatggctcttcatgtctttgatgatataatgaggatgactgagaacttgagtgagaaatacatcattggatacggggcatcaagtactgtttataaatgtgttctaaagaattgcaaaccagtggcaataaaaaagctgtatgcccactacctgcagagccttaaggaatttgaaactgagctcgagactgttggtagcatcaaacaccggaatctagtcagcctgcaagggtactcgttgtcacctgttgggaacctcctcttttatgcttatatggagagtggcagcttatgggatgttttacatgaaggctcatccaagaagaacaaacttgactgggtgactcgcctacggatcgctcttggtgcagctcaaggcctcgcttaccttcaccatgactgcagcccacgaataattcaccgggacgtaaaatcaaagaatatactcctcgacaaagattatgaggcgcatcttacagacttcggcatcgctaagagcttatgtgtctcgaagactcacacgtcaacctacgtcatgggcactattggttacattgatcctgagtacgcccgcacctcccgcctcaacgagaagtctgatgtctacagctacggcatcgttctgctggagctgctgaccggcaagaagccagtggacaacgagtgcaatctccatcacttgatcctatcgaagacggcgagcaacgaggtcatggagacggtggaccccgacgtgggagacacctgcaaggacctgggcgaggtgaagaagctgttccagctggcgctcctctgcaccaagcggcagccctcggaccggccgacgatgcacgaggtggtgcgcgtccttgactgcctggtgaacccggagccgccgccgcagccgcagcagcagcagcagaaggcgcacgcgcaccaccagctgccgccgcagccgtcgccgccggcctacgtcgacgagtacgtcagcctgcggggcaccggcgccctctcctgcgccaactcgtccagcacctcggacgccgagctgttcctcaagttcggcgaggccatctcgcagaacatggtgtaggggaagacgtagacgttcggtgaggcgccttgagtgaggccgattgcagggagtagtttgactgacattttgtgggacgcagcgcaggagattaacatgggactcagtagctagggtgttgttagctgtaaaaagtcatgtgacgcaacgcaagagcagcggagcttcttcctcatcttctttatcccccctctccatttttctttggtggtctaacttactaggaggctgtattgatccatcatcatctctcccgttcctcttccttatgatcttgtgactttc

SEQ ID NO: 45 - protein, 675 aa, maize ERECTA
MQVNRLTGSIPPELGNMSTLHYLELNDNQLTGSIPPELGRLTGLFDLNLANNHLEGPIPDNLSSCVNLNSFNAYGNKLNGTIPRSLRKLESMTYLNLSSNFISGSIPIELSRINNLDTLDLSCNMMTGPIPSSIGNLEHLLRLNLSKNDLVGFIPAEFGNLRSVMEIDLSYNHLGGLIPQELGMLQNLMLLKLENNNITGDVSSLMNCFSLNILNVSYNNLAGAVPTDNNFTRFSHDSFLGNPGLCGYWLGSSCRSTGHRDKPPISKAAIIGVAVGGLVILLMILVAVCRPHHPPAFKDATVSKPVSNGPPKLVILHMNMALHVFDDIMRMTENLSEKYIIGYGASSTVYKCVLKNCKPVAIKKLYAHYLQSLKEFETELETVGSIKHRNLVSLQGYSLSPVGNLLFYAYMESGSLWDVLHEGSSKKNKLDWVTRLRIALGAAQGLAYLHHDCSPRIIHRDVKSKNILLDKDYEAHLTDFGIAKSLCVSKTHTSTYVMGTIGYIDPEYARTSRLNEKSDVYSYGIVLLELLTGKKPVDNECNLHHLILSKTASNEVMETVDPDVGDTCKDLGEVKKLFQLALLCTKRQPSDRPTMHEVVRVLDCLVNPEPPPQPQQQQQKAHAHHQLPPQPSPPAYVDEYVSLRGTGALSCANSSSTSDAELFLKFGEAISQNMV

SEQ ID NO: 46 - protein, 3 aa, Unknown
  Group conserved across various species
STA

SEQ ID NO: 47 - protein, 4 aa, Unknown
  Group conserved across various species
NEQK

SEQ ID NO: 48 - protein, 4 aa, Unknown
  Group conserved across various species
NHQK

SEQ ID NO: 49 - protein, 4 aa, Unknown
  Group conserved across various species
NDEQ

SEQ ID NO: 50 - protein, 4 aa, Unknown
  Group conserved across various species
QHRK

SEQ ID NO: 51 - protein, 4 aa, Unknown
  Group conserved across various species
MILV

SEQ ID NO: 52 - protein, 4 aa, Unknown
  Group conserved across various species
MILF

SEQ ID NO: 53 - protein, 2 aa, Unknown
  Group conserved across various species
HY

SEQ ID NO: 54 - protein, 3 aa, Unknown
  Group conserved across various species
FYW

SEQ ID NO: 55 - protein, 3 aa, Unknown
  Group conserved across various species
CSA

SEQ ID NO: 56 - protein, 3 aa, Unknown
  Group conserved across various species
ATV

SEQ ID NO: 57 - protein, 3 aa, Unknown
  Group conserved across various species
SAG

SEQ ID NO: 58 - protein, 4 aa, Unknown
  Group conserved across various species
STNK

SEQ ID NO: 59 - protein, 4 aa, Unknown
  Group conserved across various species
STPA

SEQ ID NO: 60 - protein, 4 aa, Unknown
  Group conserved across various species
SGND

SEQ ID NO: 61 - protein, 6 aa, Unknown
  Group conserved across various species
SNDEQK

SEQ ID NO: 62 - protein, 6 aa, Unknown
  Group conserved across various species
NDEQHK

SEQ ID NO: 63 - protein, 6 aa, Unknown
  Group conserved across various species
NEQHRK

SEQ ID NO: 64 - protein, 5 aa, Unknown
  Group conserved across various species
FVLIM

SEQ ID NO: 65 - protein, 3 aa, Unknown
  Group conserved across various species
HFY